(19) United States
(12) Patent Application Publication
(10) Pub. No.: US 2024/0065836 A1
(43) Pub. Date: Feb. 29, 2024

(54) DELIVERY DEVICES FOR HEART VALVE TREATMENT DEVICES

(71) Applicant: EDWARDS LIFESCIENCES CORPORATION, Irvine, CA (US)

(72) Inventors: Michael J. Popp, Irvine, CA (US); Nicolas Schleiger, Costa Mesa, CA (US); Kevin Gantz, Laguna Beach, CA (US); George Lee Matlock, Tehachapi, CA (US); Aric Daniel Stone, Newport Beach, CA (US); Eric Robert Dixon, Villa Park, CA (US); Charles Henry Bloodworth, IV, Irvine, CA (US); Gregory Scott Tyler, II, Winston-Salem, NC (US); Asher L. Metchik, Rolling Hills Estates, CA (US); Robert Bowes, Trabuco Canyon, CA (US); Waina Michelle Chu, Tustin, CA (US); Zachary James Zira, Irvine, CA (US); Steven Park, Santa Ana, CA (US)

(21) Appl. No.: 18/496,817

(22) Filed: Oct. 27, 2023

Related U.S. Application Data
(63) Continuation of application No. PCT/US2022/025390, filed on Apr. 19, 2022.
(60) Provisional application No. 63/268,845, filed on Mar. 3, 2022.
(60) Provisional application No. 63/181,120, filed on Apr. 28, 2021.

(51) Int. Cl. A61F 2/24; A61M 25/00; A61M 25/01

(52) U.S. Cl. CPC A61F 2/2436; A61M 25/0023; A61M 25/0053; A61M 25/0136; A61M 2025/0004; A61M 2025/0047; A61M 2205/0222

(57) ABSTRACT

Components for valve treatment systems are disclosed. Valve treatment systems can include a delivery system for an implantable device. The delivery system can include one or more of clasp control components slidably disposed on a catheter handle, a control element for opening and closing the implantable device, a catheter assembly with features to reduce friction with another catheter assembly, grips for attaching catheter assemblies to clamps, catheter assemblies with features that stiffen or provide variable stiffness, and catheter assemblies with one or more steering control lumens incorporated into a reinforcement layer.

### RELATED APPLICATIONS

The present application is a continuation of Patent Cooperation Treaty Application No. PCT/US2022/025390, filed on Apr. 19, 2022, which claims the benefit of U.S. Provisional Patent Application No. 63/181,120, filed on Apr. 28, 2021, titled “Delivery Devices for Heart Valve Treatment Devices,” and U.S. Provisional Patent Application No. 63/268,845, filed on Mar. 3, 2022, titled “Delivery Devices for Heart Valve Treatment Devices,” all of which are incorporated herein by reference in their entireties for all purposes.

### BACKGROUND

The native heart valves (i.e., the aortic, pulmonary, tricuspid, and mitral valves) serve critical functions in assuring the forward flow of an adequate supply of blood through the cardiovascular system. These heart valves can be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant devices in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus can form a “D”-shaped, oval, or otherwise out-of-round cross-sectional shape having major and minor axes. The anterior leaflet can be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation can have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation can be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure can be included in the examples summarized here.

The present disclosure discloses components for treatment systems (e.g., valve treatment systems). For example, the treatment systems herein can include a delivery system and an implantable device or implant (e.g., a valve repair or replacement device). While not required, these components can make the delivery system easier to use, more ergonomic, more intuitive, and/more accurate than previous delivery systems. One or more of these components can be used with existing delivery systems. Any combination or subcombination of the disclosed components can be used together, but there is no requirement that any of the components disclosed by the present application be used with any other component disclosed by the present application.

In some implementations, a catheter assembly for controlling a transvascular implantable device includes a handle (which can comprise a handle housing) and a sheath or shaft (e.g., a catheter shaft, tube with a lumen, etc.) that extends distally from the handle housing. The catheter assembly can also include an actuation element (e.g., actuation wire, actuation shaft, actuation rod, actuation tube, etc.) and a control element (e.g., a control knob, button, switch, slider, motor, combination of these, etc.) to control, actuate, and/or move the actuation element. The control element can be coupled to the actuation element (e.g., actuation wire, actuation shaft, etc.) directly or indirectly. The actuation element can extend through the sheath and be configured to be coupled to the implantable device.

In some implementations, the control element is a control knob that is rotatable relative to the handle housing. Rotation of the control knob causes axial movement of the actuation element with respect to the handle housing and the sheath.

In some implementations, the catheter assembly includes one or more clasp control lines and one or more clasp control members (e.g., a pair of clasp control lines and a pair of clasp control members, etc.).

In some implementations, the one or more actuation lines (e.g., pair of clasp actuation lines) extend through the sheath. Each clasp actuation line is configured to be coupled to the implantable device. Each clasp control member can be axially and/or slidably movable along the handle housing. Movement (e.g., axial movement) of each clasp control member causes a clasp of the implantable device to be moved between an open configuration and a closed configuration.

In some implementations, a delivery system, such as a delivery system for delivering an implantable device, includes a first catheter assembly (e.g., a steerable catheter assembly, etc.) and a second catheter assembly (e.g., an implant catheter assembly, etc.). The first catheter assembly includes a handle and a sheath or shaft. The sheath or shaft extends from the handle in an axial direction. In some implementations, the sheath or shaft of the first catheter assembly has a distal end portion that is steerable.

In some implementations, the second catheter assembly includes a handle or handle housing and a sheath or shaft (e.g., a catheter shaft, tube with a lumen, etc.) extending distally from the handle or handle housing. The second catheter assembly can further include an actuation element (e.g., actuation wire, actuation shaft, actuation rod, actuation tube, etc.) and a control element (e.g., a control knob, button, switch, slider, motor, combination of these, etc.). The control element can be coupled to the actuation element directly or indirectly. The actuation element can extend through the sheath and can be configured to be coupled to the implantable device.

In some implementations, the control element is a control knob that is rotatable relative to the handle housing. Rotation of the control knob causes axial movement of the actuation element with respect to the handle housing and the sheath.

In some implementations, the second catheter assembly includes one or more clasp control lines (e.g., a pair of clasp control lines, etc.) and one or more clasp control members (e.g., a pair of clasp control members).

In some implementations, the one or more clasp actuation lines (e.g., pair of clasp actuation lines) extend through the sheath or shaft. Each clasp actuation line is configured to be coupled to the implantable device. In some implementations, each clasp control member is configured to be axially and/or slidably movable along the handle housing. Movement (e.g., axial movement) of each clasp control member causes a clasp of the implantable device to be moved between an open configuration and a closed configuration. The sheath of the second catheter assembly (e.g., implant catheter assembly, etc.) can extend through the first catheter assembly (e.g., steerable catheter assembly). In some implementations, the clasp control members are configured to extend around more than half, more than 90%, all, or substantially all of the handle housing, such that they can be readily actuated by an end user in any rotational orientation of the handle housing.

In some implementations, an implantable device is coupled to an actuation element and a pair of clasp actuation lines. The actuation element and the pair of clasp actuation lines extend from a distal end of a sheath or shaft of a first catheter assembly (e.g., an implant catheter assembly, etc.). The sheath or shaft is coupled at a proximal end to a handle of the first catheter assembly. In some methods, the sheath or shaft of the first catheter assembly is advanced through a second catheter assembly (e.g., a steerable catheter assembly, etc.) to position the implantable device at a delivery site.

The method can further comprise actuating a control element (e.g., knob, button, switch, slider, motor, combination of these, etc.) on the handle to cause axial movement of the actuation element to move the implantable device from a fully elongated configuration to an open configuration (e.g., a partially-open configuration as shown in FIGS. 30-31) or capture-ready configuration. The method can further comprise actuating the control element on the handle to cause axial movement of the actuation element to move the implantable device from the open configuration or capture-ready configuration to a closed configuration.

In some implementations, the control element is a knob, and actuation of the control element comprises rotating the knob with respect to a housing of the handle of the implant catheter assembly. The rotation of the knob causes axial movement of the actuation element to move the implantable device from a fully elongated configuration to an open configuration or capture ready configuration. The method can further include rotating the knob on the handle with respect to the housing of the handle to cause axial movement of the actuation element to move the implantable device from the open configuration or capture-ready configuration to a closed configuration.

In some implementations, rotating the knob to move the implantable device from the fully elongated configuration to the open configuration and rotating the knob to move the implantable device from the open configuration to the closed configuration each comprise rotating the knob in a clockwise direction.

In some implementations, rotating the knob to move the implantable device from the open configuration to the closed configuration comprises rotating the knob until an audible indication is provided by the catheter assembly.

In some implementations, actuating the control element causes axial movement of a release knob from a retracted position to an extended position with respect to the housing of the handle, wherein the release knob is coupled to the actuation element.

In some implementations, decoupling the implantable device comprises rotation of the release knob is effective to rotate the actuation element with respect to the implantable device, thereby decoupling the implantable device from the actuation element.

In some implementations, the method further comprises sliding one or more clasp control members on the handle proximally to cause axial movement of the one or more clasp actuation lines to open one or more clasps (e.g., a pair of clasps) on the implantable device.

In some implementations, the method further comprises sliding one or more clasp control members on the handle distally to cause axial movement of the pair of clasp actuation lines to close a pair of clasps on the implantable device.

In some implementations, sliding the pair of clasp control members on the handle comprises sliding each clasp control member of the pair of clasp control members independent of the other clasp control member of the pair of clasp control members.

The method can further comprise decoupling the implantable device from the actuation element and the pair of clasp actuation lines.

In some implementations, decoupling the implantable device comprises releasing a first end of each of the pair of clasp actuation lines and pulling a second end of each of the pair of clasp actuation lines to cause the first end of each of the pair of clasp actuation lines to be pulled through the sheath of the catheter assembly, thereby decoupling the implantable device from the pair of clasp actuation lines.

The above method(s) can be performed on a living animal or on a simulation, such as on a cadaver, cadaver heart, simulator (e.g., with body parts, heart, tissue, etc. being simulated), etc.

In some implementations, a delivery system, such as a delivery system for delivering an implantable device, includes one or more catheter assemblies. In some implementations the delivery system includes a first catheter assembly (e.g., an implant catheter assembly, etc.) and a second catheter assembly (e.g., a steerable catheter assembly). The first catheter assembly includes a handle, a nose grip, and a sheath. The handle of the first catheter assembly has a plurality of control members positioned thereon.

In some implementations, a first nose grip having a distal flange is disposed at a distal end of the handle of the first catheter assembly has a passage. The distal flange can be configured to have an outer diameter that is greater than an outer diameter of a central portion of the first nose grip. The sheath of the first catheter assembly extends distally from the handle and through the passage of the first nose grip.

In some implementations, the second catheter assembly includes a handle, a second nose grip, and a sheath. The second nose grip can be configured to have a distal flange at a distal end of the second nose grip. A proximal end of the second nose grip is connected to a distal end of the handle of the second catheter assembly. The second nose grip includes a passage or lumen. The distal flange of the second nose grip has an outer diameter that is greater than an outer diameter of a central portion of the second nose grip. The sheath of the second catheter assembly can extend distally from the handle and through the passage or lumen of the second nose grip. The sheath of the second catheter assembly has a distal end portion comprising a steerable section.

In some implementations, an implantable device is coupled to an adapter at a distal end of a sheath of a first catheter assembly (e.g., an implant catheter assembly, etc.). The sheath is coupled at a proximal end to a handle of the first catheter assembly. The sheath extends through a passage or lumen of a first nose grip. The first nose grip extends between a distal flange at a distal end and a proximal end that is connected to a distal end of the handle. In some methods, the first nose grip is coupled to a first clamp that is slidably coupled to a base plate by positioning the first nose grip within an opening of the first clamp. A second nose grip of a second catheter assembly (e.g., a steerable catheter assembly, etc.) is coupled to a second clamp slidably coupled to the base plate by positioning the second nose grip within an opening of the second clamp.

The method can also include advancing the sheath of the first catheter assembly through the second catheter assembly to position the implantable device at a delivery site. In some implementations, one or more of the first catheter assembly and the second catheter assembly are rotated relative to the base plate to position the implantable device at the delivery site.

In some implementations, the shaft of the first catheter assembly has a friction fit within at least one of a handle of the second catheter assembly and a shaft of the second catheter assembly.

In some implementations, the method includes (i) positioning a first locking knob in an unlocked position in which the first clamp is slidable with respect to the base plate, (ii) positioning a second locking knob in an unlocked position in which the second clamp is slidable with respect to the base plate, and (iii) axially moving the handle of the first catheter assembly effective to cause the first clamp and the second clamp to slide with respect to the base plate.

In some implementations, the method includes positioning the second locking knob in a locked position in which the second clamp is inhibited from sliding with respect to the base plate, and axially moving the handle of the first catheter assembly effective to cause the first clamp to slide with respect to the base plate and to move the shaft of the first catheter assembly axially with respect to the shaft of the second catheter assembly.

In some implementations, the method includes positioning the first locking knob in a locked position in which the first clamp is inhibited from sliding with respect to the base plate.

In some implementations, the first nose grip has a first outer diameter, the second nose grip has a second outer diameter, and the opening of the first clamp and the opening of the second clamp are sized identically. In some implementations, the first outer diameter is different than the second outer diameter.

In some implementations, the second outer diameter is greater than the first outer diameter such that rotating the first catheter assembly relative to the base plate has a different tactile feel as compared to rotating the second catheter assembly relative to the base plate.

In some implementations, the first nose grip is rotatable relative to the first catheter assembly and the second nose grip is rotatable relative to the second catheter assembly, and rotating at least one of the first catheter assembly and the second catheter assembly relative to the base plate to position the implantable device at the delivery site comprises rotating at least one of the first catheter assembly and the second catheter assembly relative to the base plate while the first nose grip and the second nose grip remain unrotated relative to the base plate.

In some implementations, the first catheter assembly is an implant catheter assembly, and the second catheter assembly is a steerable catheter assembly.

The above method(s) can be performed on a living animal or on a simulation, such as on a cadaver, cadaver heart, simulator (e.g., with body parts, heart, tissue, etc. being simulated), etc.

In some implementations, a catheter assembly for a transvascular delivery system includes a handle housing, and a sheath. The sheath extends longitudinally from the handle. A proximal portion of the sheath is stiffened with respect to the distal portion of the sheath.

In some implementations, a delivery system, such as a delivery system for delivering an implantable device, includes a first catheter assembly (e.g., a steerable catheter assembly, etc.) and a second catheter assembly (e.g., an implant catheter assembly). The first catheter assembly has a handle and a sheath extending from the handle in an axial direction. The sheath comprises a proximal portion and a distal portion comprising a steerable section. The second catheter assembly has a handle and a sheath comprising a proximal portion and a distal portion. The sheath is extendable or configured to extend coaxially through the sheath of the first catheter assembly. At least one of the proximal portion of the sheath of the first catheter assembly and the proximal portion of the sheath of the second catheter assembly is stiffened relative to the distal portion of the sheath of the first catheter assembly or the distal portion of the sheath of the second catheter assembly, respectively.

In some implementations, a catheter assembly for a transvascular delivery system includes a handle housing and a sheath. The sheath extends distally from the handle housing between a proximal end and a distal end. The sheath has a first outer diameter along a first length of the sheath and a second outer diameter along a second length of the sheath.

In some implementations, a catheter assembly for a transvascular delivery system comprises a handle housing and a sheath (e.g., catheter, tube, etc.) extending longitudinally from the handle. The sheath has a proximal portion and a distal portion. The proximal portion of the sheath is configured to have a different stiffness comparted to the distal portion of the sheath.

In some implementations, the proximal portion of the sheath comprises a material having a higher durometer than materials of the distal portion of the sheath.

In some implementations, only the proximal portion of the sheath comprises a braid, mesh, or woven material. In some implementations, only the distal portion of the sheath comprises a braid, mesh, or woven material. In some implementations, both the proximal portion and the distal portion of the sheath comprise a braid, mesh, or woven material.

In some implementations, only the proximal portion of the sheath comprises at least one laser-cut hypotube. In some implementations, only the distal portion of the sheath comprises at least one laser-cut hypotube. In some implementations, both the proximal portion and the distal portion of the sheath comprise at least one laser-cut hypotube.

In some implementations, the at least one laser-cut hypotube extends through an outer jacket of the sheath.

In some implementations, the sheath is a multi-layer sheath, and the at least one laser-cut hypotube comprises a layer of the multi-layer sheath.

In some implementations, the at least one laser-cut hypotube has a stiffness that varies along a length of the at least one laser-cut hypotube.

In some implementations, the sheath comprises a steerable portion. In some implementations, the steerable portion of the sheath is in the distal portion of the sheath. In some implementations, the steerable portion of the sheath comprises one or more of a pull wire, pull ring, pull wire lumen, etc. In some implementations, pull wire, pull ring, pull wire lumen, etc. are radially inside at least one of the braid (or other mesh or woven material) and the at least one laser-cut hypotube. In some implementations, pull wire, pull ring, pull wire lumen, etc. are radially outside of at least one of the braid (or other mesh or woven material) and the at least one laser-cut hypotube.

In some implementations, the sheath is a multi-layer sheath, and the proximal portion of the sheath comprises a first layer comprising at least one laser-cut hypotube and a second layer comprising a braid (or other mesh or woven material). In some implementations, the sheath is a multi-layer sheath, and the distal portion of the sheath comprises a first layer comprising at least one laser-cut hypotube and a second layer comprising a braid (or other mesh or woven material).

In some implementations, the braid, mesh, or woven material is positioned between a lumen extending through the sheath and the at least one laser-cut hypotube.

In some implementations, the proximal portion of the sheath comprises a first laser-cut hypotube along a first length of the sheath and a second laser-cut hypotube along a second length of the sheath, wherein the first laser-cut hypotube and the second laser-cut hypotube have different stiffnesses.

In some implementations, the sheath defines a lumen extending longitudinally through the sheath, and wherein the lumen has a cross-section that transitions from having a circular shape to having a non-circular shape.

The various sheaths described in any of the implementations herein can include any of the features of the sheaths described herein.

In some implementations, a delivery system for an implantable device includes a first catheter assembly (e.g., an implant catheter assembly, etc.) and a second catheter assembly (e.g., a steerable catheter assembly, etc.). The second catheter assembly has a handle and a sheath extending from the handle in an axial direction. The sheath has a steerable section. The first catheter assembly has a handle and a sheath. The sheath of the first catheter assembly extends coaxially through the sheath of the second catheter assembly. The sheath of the first catheter assembly has a first outer diameter along a first length and a second outer diameter along a second length.

In some implementations, an implantable device is coupled to an actuation element extending from a distal end of a sheath of a first catheter assembly (e.g., an implant catheter assembly, etc.). The sheath is coupled at a proximal end to a handle of the first catheter assembly. The sheath has a first outer diameter along a first length of the sheath and a second outer diameter along a second length of the sheath. In some methods, the sheath of the first catheter assembly is advanced through a second catheter assembly (e.g., a steerable catheter assembly, etc.) to position the implantable device at a delivery site.

In some implementations, a position of the implantable device relative to the delivery site is adjusted by extending the distal end of the sheath of the first catheter assembly relative to a distal end of the sheath of the second catheter assembly. During the adjusting, the second length of the sheath is positioned coaxially within the steerable section of the sheath of the second catheter assembly.

The above method(s) can be performed on a living animal or on a simulation, such as on a cadaver, cadaver heart, simulator (e.g., with body parts, heart, tissue, etc. being simulated), etc.

In some implementations, a method of delivering an implantable device comprises obtaining a first catheter assembly coupled to the implantable device and advancing a sheath (e.g., catheter, tubing, lumen, etc.) of the first catheter assembly through a second catheter assembly to position the implantable device at a delivery site. In some implementations, the first catheter assembly comprises an actuation element extendable from a distal end of the sheath, wherein the actuation element is coupled to the implantable device.

In some implementations, the sheath is coupled at a proximal end to a handle of the first catheter assembly.

In some implementations, the sheath has a first outer diameter along a first length of the sheath and a second outer diameter along a second length of the sheath. In some implementations, the first length is between the second length of the sheath and the proximal end of the sheath of the first catheter assembly.

In some implementations, the sheath of the second catheter assembly comprises a steerable section.

In some implementations, the method includes adjusting a position of the implantable device relative to the delivery site by extending the distal end of the sheath of the first catheter assembly relative to a distal end of the sheath of the second catheter assembly. In some implementations, during the adjusting, the second length of the sheath is positioned coaxially within the steerable section of the sheath of the second catheter assembly.

In some implementations, the method includes decoupling the implantable device from the actuation element.

In some implementations, the second outer diameter is smaller than the first outer diameter. In some implementations, a difference between the first outer diameter and the second outer diameter is from about 0.25 to about 0.76 mm. In some implementations, a transition from the first outer diameter to the second outer diameter forms a smooth taper over a distance of from about 25 mm to about 50 mm.

In some implementations, the sheath includes a lubricated coating on an outer surface of the sheath along the second length of the sheath. In some implementations, the lubricated coating comprises a hydrophilic coating.

In some implementations, the first catheter assembly is an implant catheter assembly, and the second catheter assembly is a steerable catheter assembly.

The above method(s) can be performed on a living animal or on a simulation, such as on a cadaver, cadaver heart, simulator (e.g., with body parts, heart, tissue, etc. being simulated), etc.

In some implementations, a catheter assembly for controlling an implantable device comprises a handle housing, a sheath (e.g., catheter, tube, etc.) extending distally from the handle housing, and an actuation element extending through the sheath, the actuation element configured to be coupled to the implantable device. In some implementations, the catheter assembly further includes a control element coupled to the actuation element, wherein actuation of the control element causes axial movement of the actuation element with respect to the handle housing and the sheath.

In some implementations, the catheter assembly further comprises a pair of clasp actuation lines (e.g., a first clasp actuation line and a second clasp actuation line) extending through the sheath, each clasp actuation line (e.g., the first clasp actuation line and/or the second clasp actuation line) of the pair of clasp actuation lines is configured to be coupled to the implantable device.

In some implementations, the catheter assembly further comprises a first clasp control member, wherein actuation of the first clasp control member causes a first clasp of the implantable device to be moved between an open configuration and a closed configuration. In some implementation, the first clasp control member is physically or operatively coupled to the first clasp via one of the pair of clasp actuation lines (e.g., a first clasp actuation line) and such that actuation of the first clasp control member can cause axial movement of the clasp actuation line to move the first clasp between the open configuration and the closed configuration. The first clasp control member can be configured in a variety of ways, e.g., as a knob, slider, latch, switch, button, gear, etc. In some implementations, the first clasp control member is configured to extend around between 45-90% of a circumference of the handle housing, between 60-90% of a circumference of the handle housing, between 75-90% of a circumference of the handle housing, between 85-90% of a circumference of the handle housing, or 90% (or substantially 90%) of a circumference of the handle housing. or substantially 90% of a circumference of the handle housing.

In some implementations, the first clasp control member is axially movable relative to the handle housing and the sheath, and axial movement of the first clasp control member causes the first clasp of the implantable device to be moved between the open configuration and the closed configuration. In some implementations, the first clasp control member is axially movable relative to the handle housing and the sheath, and axial movement of the first clasp control member causes axial movement of one of the pair of clasp actuation lines (e.g., the first clasp actuation line) such that the first clasp of the implantable device is moved between the open configuration and the closed configuration.

In some implementations, the catheter assembly further comprises a second clasp control member, wherein actuation of the second clasp control member causes a second clasp of the implantable device to be moved between an open configuration and a closed configuration. In some implementation, the second clasp control member is physically or operatively coupled to the second clasp via one of the pair of clasp actuation lines (e.g., a second clasp actuation line) and such that actuation of the second clasp control member can cause axial movement of the clasp actuation line to move the second clasp between the open configuration and the closed configuration. The second clasp control member can be configured in a variety of ways, e.g., as a knob, slider, latch, switch, button, gear, etc. In some implementations, the second clasp control member is configured to extend around between 45-90% of a circumference of the handle housing, between 60-90% of a circumference of the handle housing, between 75-90% of a circumference of the handle housing, between 85-90% of a circumference of the handle housing, or 90% (or substantially 90%) of a circumference of the handle housing.

In some implementations, both of the first clasp control member and the second clasp control member are configured to extend around between 60-90% of a circumference of the handle housing, between 75-90% of a circumference of the handle housing, between 85-90% of a circumference of the handle housing, or 90% (or substantially 90%) of a circumference of the handle housing such that they can be readily actuated by an end user in any rotational orientation of the handle housing. In some implementations, both of the first clasp control member and the second clasp control member are configured to extend 90% (or substantially 90%) of a circumference of the handle housing such that together they encircle the handle housing such that they can be readily actuated by an end user in any rotational orientation of the handle housing.

In some implementations, the second clasp control member is axially movable relative to the handle housing and the sheath, and wherein axial movement of the second clasp control member causes the second clasp of the implantable device to be moved between the open configuration and the closed configuration. In some implementations, the second clasp control member is axially movable relative to the handle housing and the sheath, and axial movement of the second clasp control member causes axial movement of one of the pair of clasp actuation lines (e.g., the second clasp actuation line) such that the second clasp of the implantable device is moved between the open configuration and the closed configuration.

In some implementations, the catheter assembly includes a slide lock configured to slide between (i) a first position in which the slide lock is not coupled to at least one of the first clasp control member and the second clasp control member, and (ii) a second position in which the slide lock is coupled to both the first clasp control member and the second clasp control member. In some implementations, when the slide lock is in the first position, the first clasp control member is actuatable independently of the second clasp control member, and when the slide lock is in the second position, the first clasp control member and the second clasp control member are coupled such that actuation of the first clasp member also actuates the second clasp member.

In some implementations, the first clasp control member and the second clasp control member are actuatable by axial movement thereof, and wherein when the slide lock is in the first position, the first clasp control member is axially moveable independently of the second clasp control member, and when the slide lock is in the second position, the first clasp control member and the second clasp control member are coupled such that the first clasp member and the second clasp member are configured to move axially together when actuated.

In some implementations, the handle housing comprises a first detent at a first axial position along a path of one of the pair of clasp control members to maintain the one of the pair of clasp control members in a proximal position and a second detent at a second axial position along the path of one of the pair of clasp control members to maintain the one of the pair of clasp control members in a distal position.

In some implementations, the catheter assembly further comprises an externally threaded retractor coupled to the control element and the actuation element, wherein the externally threaded retractor is rotationally fixed with respect to the handle housing, and wherein actuation of the control element advances the externally threaded retractor in an axial direction, thereby causing linear movement of the actuation element.

In some implementations, the catheter assembly further comprises a clutch spring positioned about the externally threaded retractor and configured to bias the externally threaded retractor distally towards threads of an internally threaded tube within the handle housing.

In some implementations, the control element is a control knob and rotation of the control knob causes rotation of the internally threaded tube with respect to the handle housing, which drives the externally threaded retractor to a proximal position in which external threads of the externally threaded retractor and internal threads of the internally threaded tube disengage, and wherein continued rotation of the control knob provides an audible indication.

In some implementations, a catheter assembly includes a handle housing and a sheath. The sheath extends longitudinally from the handle. The sheath comprises a woven layer. A lumen is interwoven into the woven layer. A flex control element extends through the lumen.

In some implementations, the flex control element can comprise a wire. The lumen can comprise a metal tube. The lumen can extend in a direction of a length of the sheath and/or the lumen can have a spiral configuration.

Other features, elements, and components from any of the various implementations and examples herein can also be included in the catheter assembly mutatis mutandis.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of some examples and implementations will be made by reference to various aspects of the appended drawings. It is appreciated that these drawings depict only example implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing mitral regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example implantable device or implant of FIGS. 8-14 being delivered and implanted within a native valve;

FIG. 22 shows a perspective view of an example implantable device or implant in a closed position;

FIG. 23 shows a front view of the implantable device or implant of FIG. 22;

FIG. 24 shows a side view of the implantable device or implant of FIG. 22;

FIG. 25 shows a front view of the implantable device or implant of FIG. 22 with a cover covering the paddles and a coaptation element or spacer;

FIG. 26 shows a top perspective view of the implantable device or implant of FIG. 22 in an open position;

FIG. 27 shows a bottom perspective view of the implantable device or implant of FIG. 22 in an open position;

FIG. 28 shows a clasp for use in an implantable device or implant;

FIG. 29 shows a portion of native valve tissue grasped by a clasp;

FIG. 30 shows a side view of an example implantable device or implant in a partially-open position with clasps in a closed position;

FIG. 31 shows a side view of an example implantable device or implant in a partially-open position with clasps in an open position;

FIG. 32 shows a side view of an example implantable device or implant in a half-open position with clasps in a closed position;

FIG. 33 shows a side view of an example implantable device or implant in a half-open position with clasps in an open position;

FIG. 34 shows a side view of an example implantable device or implant in a three-quarters-open position with clasps in a closed position;

FIG. 35 shows a side view of an example implantable device or implant in a three-quarters-open position with clasps in an open position;

FIG. 36 shows a side view of an example implantable device in a fully open or fully elongated position with clasps in a closed position;

FIG. 37 shows a side view of an example implantable device in a fully open or fully elongated position with clasps in an open position;

FIGS. 38-49 show the example implantable device or implant of FIGS. 30-38, including a cover, being delivered and implanted within a native valve;

FIG. 50 is a schematic view illustrating a path of native valve leaflets along each side of a coaptation element or spacer of an example valve repair device or implant;

FIG. 51 is a top schematic view illustrating a path of native valve leaflets around a coaptation element or spacer of an example valve repair device or implant;

FIG. 52 illustrates a coaptation element or spacer in a gap of a native valve as viewed from an atrial side of the native valve;

FIG. 53 illustrates a valve repair device or implant attached to native valve leaflets with the coaptation element or spacer in the gap of the native valve as viewed from a ventricular side of the native valve;

FIG. 54 is a perspective view of a valve repair device or implant attached to native valve leaflets with the coaptation element or spacer in the gap of the native valve shown from a ventricular side of the native valve;

FIG. 55 shows a perspective view of an example implantable device or implant in a closed position;

FIG. 56 shows a perspective view of an example clasp of an example implantable device or implant in a closed position;

FIG. 57 illustrates a valve repair device with paddles in an open position;

FIG. 58 illustrates the valve repair device of FIG. 57, in which the paddles are in the open position and gripping members are moved to create a wider gap between the gripping members and paddles;

FIG. 59 illustrates the valve repair device of FIG. 57, in which the valve repair device is in the position shown in FIG. 57 with valve tissue placed between the gripping members and the paddles;

FIG. 60 illustrates the valve repair device of FIG. 57, in which the gripping members are moved to lessen the gap between the gripping members and the paddles;

FIGS. 61A-61B illustrate the movement of the paddles of the valve repair device of FIG. 57 from the open position to a closed position;

FIG. 62 illustrates the valve repair device of FIG. 57 in a closed position, in which the gripping members are engaging valve tissue;

FIG. 63 illustrates the valve repair device of FIG. 57 after being disconnected from a delivery device and attached to valve tissue, in which the valve repair device is in a closed and locked condition;

FIG. 64 illustrates a distal end of an example system or assembly including a delivery system and an implantable device;

FIG. 65 illustrates a proximal end of the example system or assembly of FIG. 64;

FIG. 66 illustrates an example implant catheter assembly for use in a delivery system coupled to an implantable device;

FIG. 67 illustrates a schematic illustration of an example implant catheter assembly coupled to an implantable device, in which each of the clasp actuation lines is coupled to a clasp control member positioned on the handle and the actuation element is coupled to a control element or knob positioned on the handle;

FIG. 68 is a perspective view of an example handle of an implant catheter assembly;

FIG. 69 is a cross-section of the handle of FIG. 68 perpendicular to the plane defined by line A-A, in which one of the clasp control tubes is bisected;

FIG. 70 is another cross-section of the handle of FIG. 68 perpendicular to the plane defined by line B-B, in which the release knob is bisected;

FIG. 70A is an enlarged portion of FIG. 70;

FIG. 71 is another cross-section of the handle of FIG. 68 perpendicular to the plane defined by line C-C, in which the release knob and the suture locks are bisected;

FIG. 71A is another perspective view of the handle of FIG. 68 in which a clasp setting spacer supports the clasp control tubes;

FIG. 72 is another perspective view of the handle of FIG. 68 in which the housing includes a pair of detents for fixing the clasp control members into position;

FIG. 73 is a perspective view of an example proximal end of a handle in which the release knob is in a distal position;

FIG. 74 is a perspective view of an example proximal end of a handle in which the release knob is in a proximal position;

FIG. 75 is a perspective view of a partial cut away of a handle including an example release knob having a ratcheting mechanism;

FIG. 76 is a perspective view of the release knob of FIG. 75 having the ratcheting mechanism;

FIG. 77 is a cross-sectional view of the release knob of FIGS. 75-76 perpendicular to the plane defined by line D-D in which the pawls are sectioned;

FIG. 78 is a cross-sectional view of an example suture lock in which a clasp actuation line is fixed to a post at one end and extends between a suture lock body and a suture lock body receptacle at a second end after having been coupled to an implantable device;

FIG. 79 is a perspective view of an example nose grip for coupling a delivery system or component thereof to a stabilization system;

FIG. 80 is a cross-sectional view of an example clamp of a stabilization system surrounding a nose grip of a delivery system or catheter assembly;

FIG. 81 is another cross-sectional view of an example clamp of a stabilization system surrounding a nose grip of a delivery system or catheter assembly;

FIG. 82A is a perspective view of an example steerable catheter assembly including a nose grip being coupled to a clamp of a stabilization system;

FIG. 82B is a perspective view of the example steerable catheter assembly of FIG. 82A upon closure of the clamp;

FIG. 82C illustrates axial movement of the steerable catheter assembly and the clamp of FIGS. 82A-82B with respect to a base plate of the stabilization system;

FIG. 83A is a perspective view of an example implant catheter assembly including a nose grip;

FIG. 83B is a perspective view of the example implant catheter assembly of FIG. 83A being coupled to a clamp of a stabilization system;

FIG. 83C is a perspective view of the example implant catheter assembly of FIGS. 83A-83B upon closure of the clamp;

FIG. 84 is a perspective view of a proximal end of a stabilization system coupled to a steerable catheter assembly and an implant catheter assembly and illustrating the axial movement of the steerable catheter assembly and implant catheter assembly with respect to the base plate of the stabilization system;

FIGS. 85A and 85B illustrate the outer diameter of the nose grips of the steerable catheter assembly and the implant catheter assembly;

FIG. 86 is a cross-section of a shaft of an implant catheter assembly having a reduced outer diameter portion and a low-friction coating positioned within a steerable portion of the shaft of a steerable catheter assembly;

FIG. 87 is another illustration of a shaft of an implant catheter assembly having a reduced outer diameter portion and a low-friction coating;

FIG. 88A illustrates a sheath of an implant catheter assembly having a reduced outer diameter positioned within a sheath of a steerable catheter assembly in a retracted position;

FIG. 88B illustrates the sheath of the implant catheter assembly of FIG. 88A positioned within the sheath of the steerable catheter assembly in an extended position;

FIG. 89A illustrates a proximal portion of a sheath of a steerable catheter assembly including a stiffening material;

FIG. 89B illustrates a proximal portion of a sheath of an implant catheter assembly including a stiffening material;

FIG. 89C illustrates a proximal portion of a sheath of a steerable catheter assembly and a proximal portion of a sheath of an implant catheter assembly including a stiffening material;

FIG. 90 illustrates a sheath of a steerable catheter assembly having a stiffened length between a proximal end of the steerable catheter sheath and a steerable portion;

FIG. 91 illustrates a steerable catheter assembly having two hypotubes having different stiffnesses;

FIG. 92 illustrates a steerable catheter assembly having a laser-cut hypotube positioned over a braid, mesh, or woven material to increase the stiffness of a portion of the steerable catheter assembly;

FIG. 93 illustrates four segments of a laser-cut hypotube;

FIG. 94 illustrates a hypotube having an interrupted spiral cut;

FIGS. 95A and 95B are radial cross-sections of example multi-layer sheaths;

FIG. 96A is a longitudinal cross-section of another example multi-layer sheath;

FIG. 96B is a longitudinal cross-section of a proximal portion of the multi-layer sheath of FIG. 96A;

FIG. 97 illustrates an example catheter assembly with portions removed to illustrate internal components;

FIG. 98 illustrates an example catheter assembly with portions removed to illustrate internal components;

FIGS. 99A, 99B, and 99C illustrate example braid patterns having a tube for accommodating a flex element woven therein;

FIG. 99D is a longitudinal cross-section of an example multi-layer sheath incorporating the braid of any one of FIGS. 99A-99C;

FIG. 99E illustrates an example braid pattern having a tube for accommodating a flex element woven therein; and

FIG. 99F is a longitudinal cross-section of an example multi-layer sheath incorporating the braid of FIG. 99E.

### DETAILED DESCRIPTION

The following description refers to the accompanying drawings, which illustrate example implementations of the present disclosure. Other implementations having different structures and operation do not depart from the scope of the present disclosure.

Example implementations of the present disclosure are directed to systems, devices, methods, etc. for repairing a defective heart valve. For example, various implementations of implantable devices, valve repair devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the techniques and methods herein can be performed on a living animal or on a simulation, such as on a cadaver, cadaver heart, simulator (e.g., with the body parts, heart, tissue, etc. being simulated), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

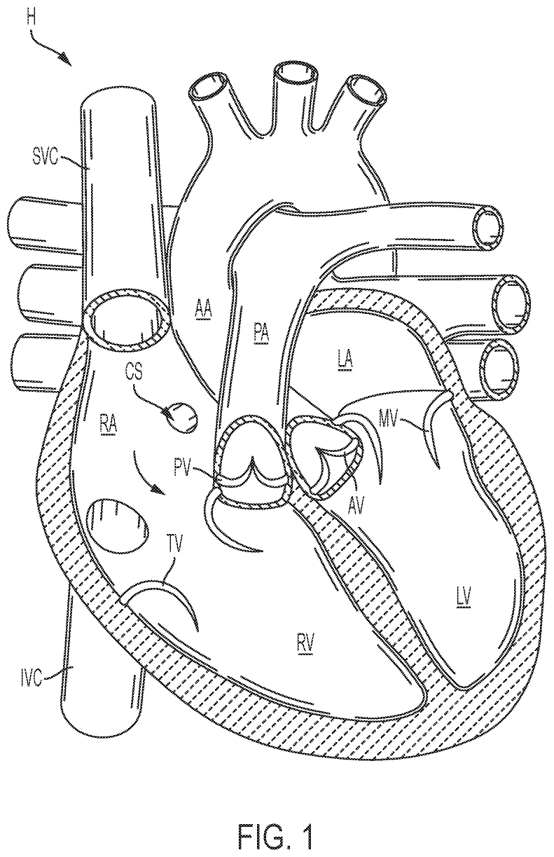

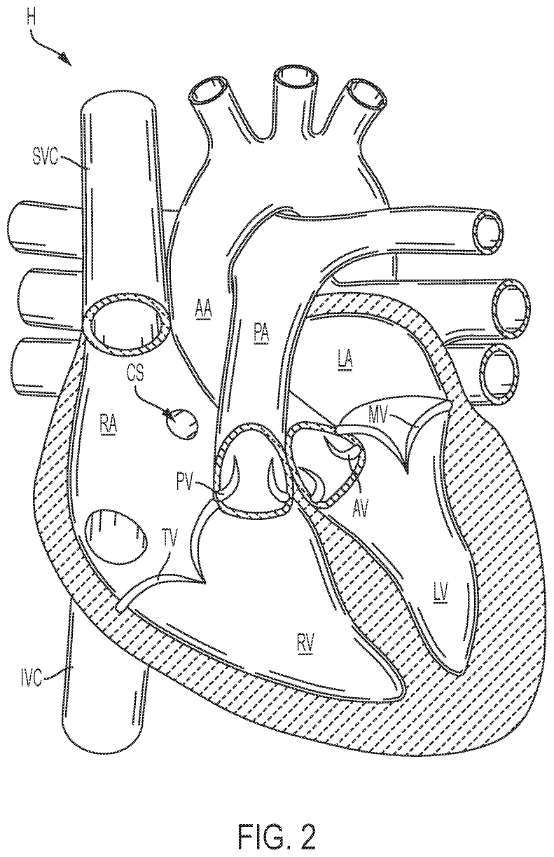

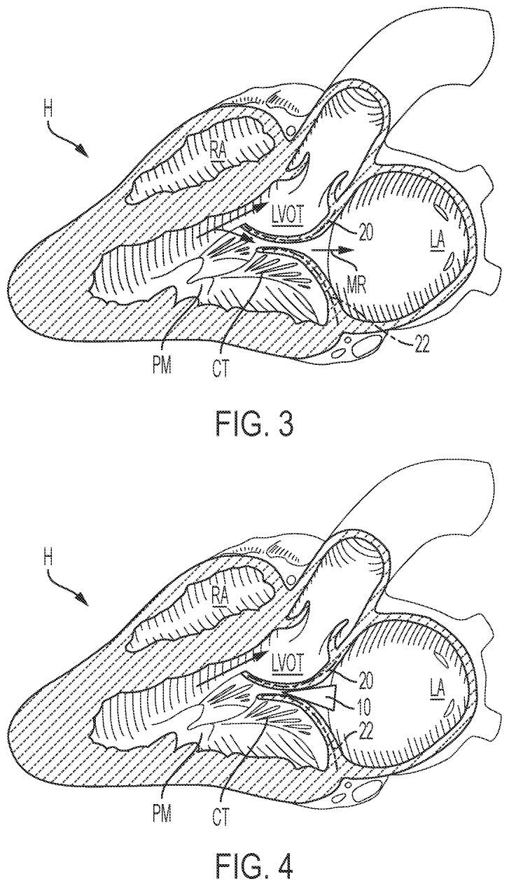

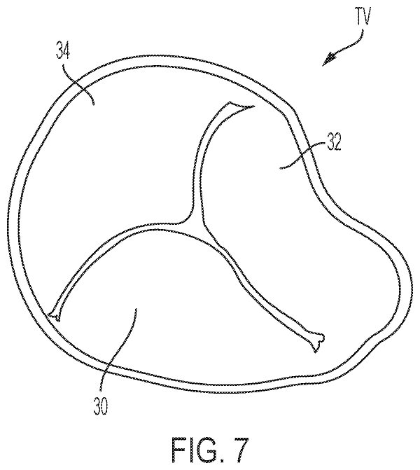

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent or inhibit blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

Referring now to FIGS. 1-7, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) can distort a native valve's geometry, which can cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant or incompetent-which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction can be caused by rheumatic disease (Ma) or dilation of a ventricle (IIIb).

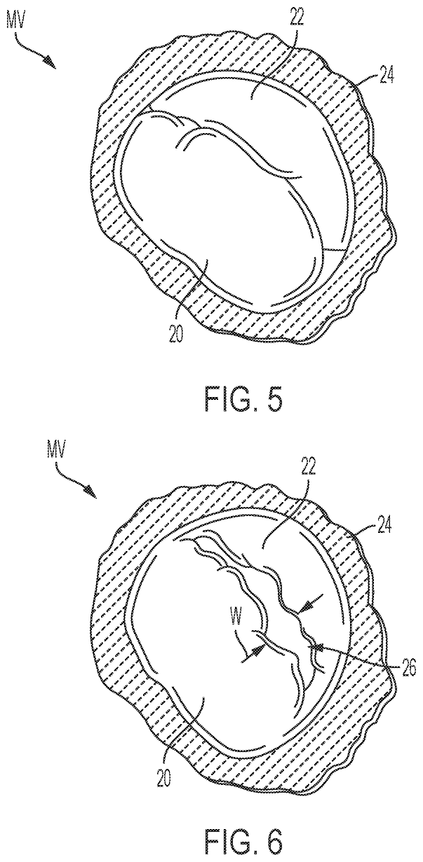

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm. As set forth above, there are several different ways that a leaflet (e.g., leaflets 20, 22 of mitral valve MV) may malfunction which can thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of an implantable device, valve repair device, or implant 10 is shown implanted between the leaflets 20, 22 such that regurgitation does not occur during systole (compare FIG. 3 with FIG. 4). In some implementations, the coaptation element (e.g., spacer, coaption element, gap filler, etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another)).

Although stenosis or regurgitation can affect any valve, stenosis is predominantly found to affect either the aortic valve AV or the pulmonary valve PV, and regurgitation is predominantly found to affect either the mitral valve MV or the tricuspid valve TV Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, prevents the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation can occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example implant or implantable device (e.g., implantable prosthetic device, etc.) can optionally have a coaptation element (e.g., spacer, coaption element, gap filler, etc.) and at least one anchor (e.g., one, two, three, or more). In some implementations, an implantable device or implant can have any combination or sub-combination of the features disclosed herein without a coaptation element. When included, the coaptation element (e.g., coaption element, spacer, etc.) is configured to be positioned within the native heart valve orifice to help fill the space between the leaflets and form a more effective seal, thereby reducing or preventing regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral valve leaflets 20, 22 or tricuspid valve leaflets 30, 32, 34) that do not close completely.

The optional coaptation element (e.g., spacer, coaption element, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surface that extends between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element, such as a shaft or actuation wire, to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element, e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still-compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for various implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

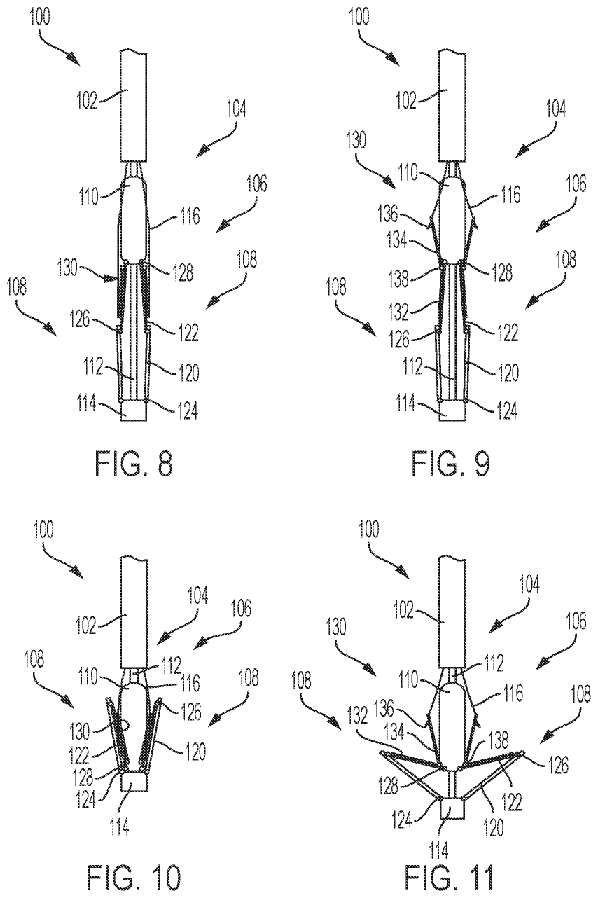

Referring now to FIGS. 8-15, a schematically illustrated implantable device or implant 100 (e.g., a prosthetic spacer device, valve repair device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for an implantable device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system 102 or other means for delivery. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 (e.g., spacer, plug, filler, foam, sheet, membrane, coaption element, etc.) that is adapted to be implanted between leaflets of a native valve (e.g., a native mitral valve, native tricuspid valve, etc.) and is slidably attached to an actuation element 112 (e.g., actuation wire, actuation shaft, actuation tube, etc.). The anchor portion 106 includes one or more anchors 108 that are actuatable between open and closed conditions and can take a wide variety of forms, such as, for example, paddles, gripping elements, or the like. Actuation of the means for actuating or actuation element 112 opens and closes the anchor portion 106 of the device 100 to grasp the native valve leaflets during implantation. The means for actuating or actuation element 112 (as well as other means for actuating and actuation elements herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and the means for coapting or coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and means for actuating or actuation element 112 (e.g., actuation wire, actuation shaft, etc.). These can be configured to extend through a guide catheter/sheath (e.g., a transseptal sheath, etc.). In some implementations, the means for actuating or actuation element 112 extends through a delivery catheter and the means for coapting or coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element removably attaches the coaptation element 110 to the delivery system 102, either directly or indirectly, so that the means for actuating or actuation element 112 slides through the collar or other attachment element and, in some implementations, through a means for coapting or coaptation element 110 during actuation to open and close the paddles 120, 122 of the anchor portion 106 and/or anchors 108.

In some implementation, the anchor portion 106 and/or anchors 108 can include attachment portions or gripping members. The illustrated gripping members can comprise clasps 130 that include a base or fixed arm 132, a moveable arm 134, optional barbs, friction-enhancing elements, or other means for securing 136 (e.g., protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate means for coapting or coaptation element 110. In some implementations, the clasps (e.g., barbed clasps, etc.) have flat surfaces and do not fit in a recess of the inner paddle. Rather, the flat portions of the clasps are disposed against the surface of the inner paddle 122. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs, friction-enhancing elements, or means for securing 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. This pinching force remains constant regardless of the position of the inner paddles 122. Optional barbs, friction-enhancing elements, or other means for securing 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a means for coapting or coaptation element 110. The clasps 130 can be used to grasp and/or further secure the native leaflets by engaging the leaflets with optional barbs, friction-enhancing elements, or means for securing 136 and pinching the leaflets between the moveable and fixed arms 134, 132. The friction-enhancing elements, or other means for securing 136 (e.g., barbs, protrusions, ridges, grooves, textured surfaces, adhesive, etc.) of the clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter in the fully open position, because the fully open position takes up the least space and allows the smallest catheter to be used (or the largest device 100 to be used for a given catheter size). In the elongated condition the cap 114 is spaced apart from the means for coapting or coaptation element 110 such that the paddles 120, 122 are fully extended. In some implementations, an angle formed between the interior of the outer and inner paddles 120, 122 is approximately 180 degrees. The clasps 130 are kept in a closed condition during deployment through the delivery system 102 so that the optional barbs, friction-enhancing elements, or other means for securing 136 (FIG. 9) do not catch or damage the delivery system 102 or tissue in the patient's heart. The actuation lines 116 can attach to the moveable arms 134.

Referring now to FIG. 9, the device 100 is shown in an elongated detangling condition, similar to FIG. 8, but with the clasps 130 in a fully open position, ranging from about 140 degrees to about 200 degrees, from about 170 degrees to about 190 degrees, or about 180 degrees between fixed and moveable portions 132, 134 of the clasps 130. Fully opening the paddles 120, 122 and the clasps 130 has been found to improve ease of detanglement or detachment from anatomy of the patient, such as the chordae tendineae CT, during implantation of the device 100.

Referring now to FIG. 10, the device 100 is shown in a shortened or fully closed condition. The compact size of the device 100 in the shortened condition allows for easier maneuvering and placement within the heart. To move the device 100 from the elongated condition to the shortened condition, the means for actuating or actuation element 112 is retracted to pull the cap 114 towards the means for coapting or coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the means for coapting or coaptation element 110 cause the paddles or gripping elements to move radially outward. During movement from the open to closed position, the outer paddles 120 maintain an acute angle with the means for actuating or actuation element 112. The outer paddles 120 can optionally be biased toward a closed position. The inner paddles 122 during the same motion move through a considerably larger angle as they are oriented away from the means for coapting or coaptation element 110 in the open condition and collapse along the sides of the means for coapting or coaptation element 110 in the closed condition. In some implementations, the inner paddles 122 are thinner and/or narrower than the outer paddles 120, and the connection portions 126, 128 (e.g., joints, flexible connections, etc.) connected to the inner paddles 122 can be thinner and/or more flexible. For example, this increased flexibility can allow more movement than the connection portion 124 connecting the outer paddle 120 to the cap 114. In some implementations, the outer paddles 120 are narrower than the inner paddles 122. The connection portions 126, 128 connected to the inner paddles 122 can be more flexible, for example, to allow more movement than the connection portion 124 connecting the outer paddle 120 to the cap 114. In some implementations, the inner paddles 122 can be the same or substantially the same width as the outer paddles

Referring now to FIGS. 11-13, the device 100 is shown in a partially open, grasp-ready condition. To transition from the fully closed to the partially open condition, the means for actuating or actuation element (e.g., actuation wire, actuation shaft, etc.) is extended to push the cap 114 away from the means for coapting or coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single means for actuating or single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. For example, the device 100 can have two actuation elements and two independent caps (or other attachment portions), such that one independent actuation element (e.g., wire, shaft, etc.) and cap (or other attachment portion) are used to control one paddle, and the other independent actuation element and cap (or other attachment portion) are used to control the other paddle.

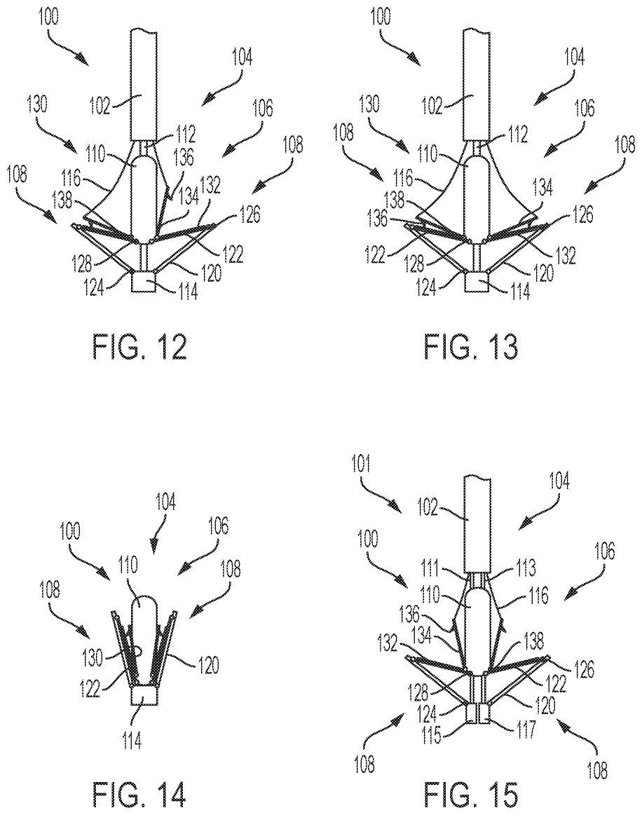

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 or means for delivery and means for actuating or actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol-produced in a wire, sheet, tubing, or laser sintered powder—and are biased to hold the outer paddles 120 closed around the means for coapting or coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device 100 illustrated by FIG. 11, except the device 101 of FIG. 15 includes an actuation element that is configured as two independent actuation elements or actuation wires 111, 113 that are coupled to two independent caps 115, 117. To transition a first inner paddle 122 and a first outer paddle 120 from the fully closed to the partially open condition, the means for actuating or actuation element 111 is extended to push the cap 115 away from the means for coapting or coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the means for actuating or actuation element 113 is extended to push the cap 115 away from the means for coapting or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single means for actuating or actuation element 112.

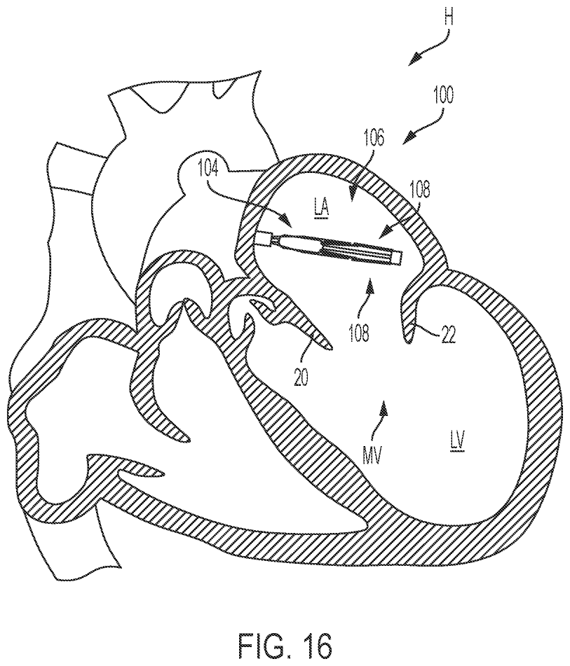

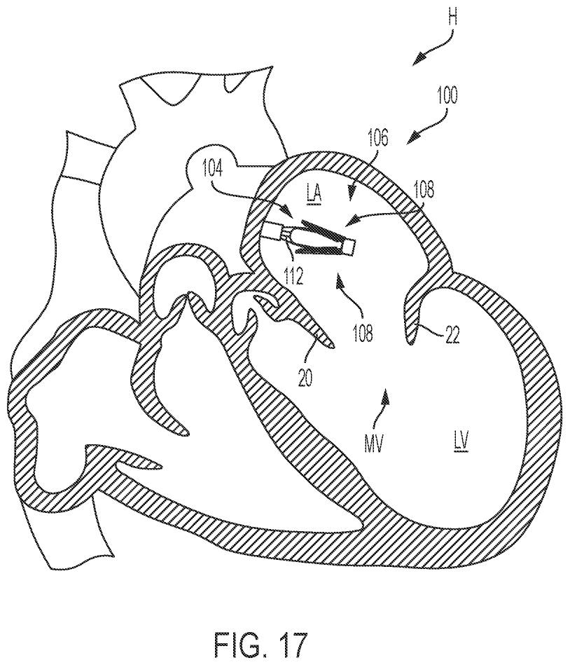

Referring now to FIGS. 16-21, the implantable device 100 of FIGS. 8-14 is shown being delivered and implanted within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The means for actuating or actuation element 112 is then retracted to move the implant/device into the fully closed condition shown in FIG. 17.

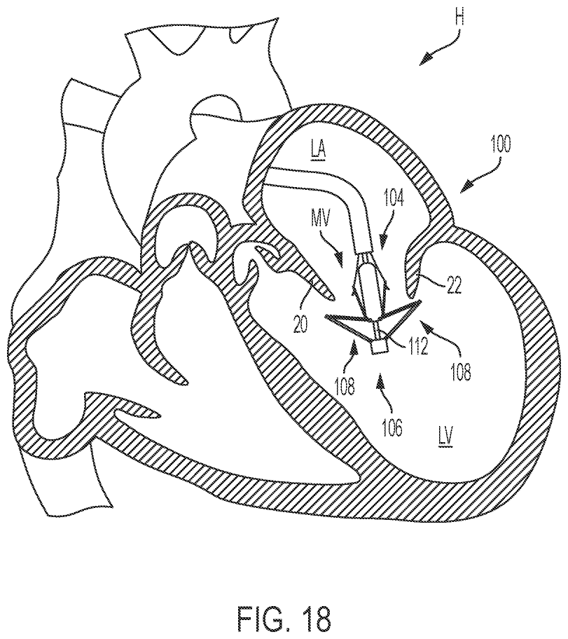

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

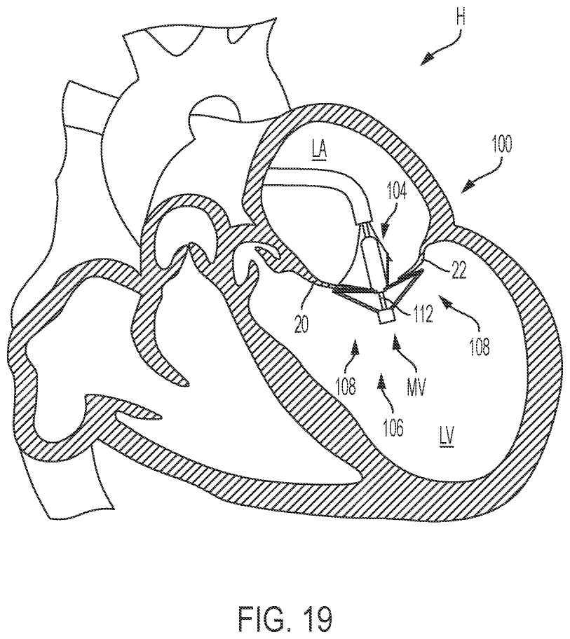

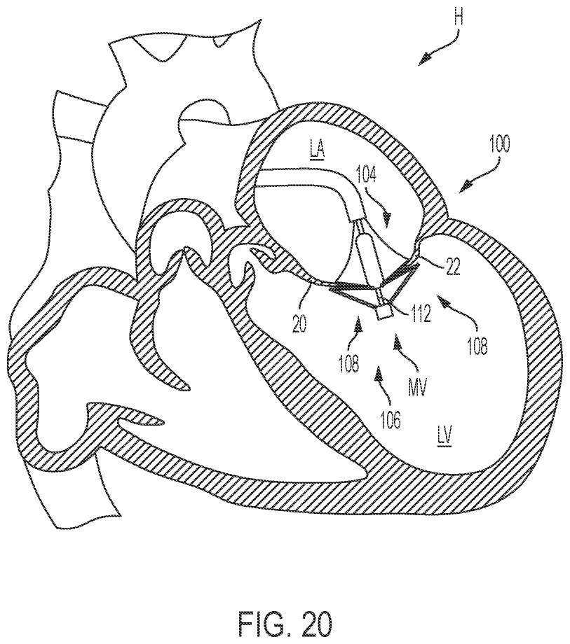

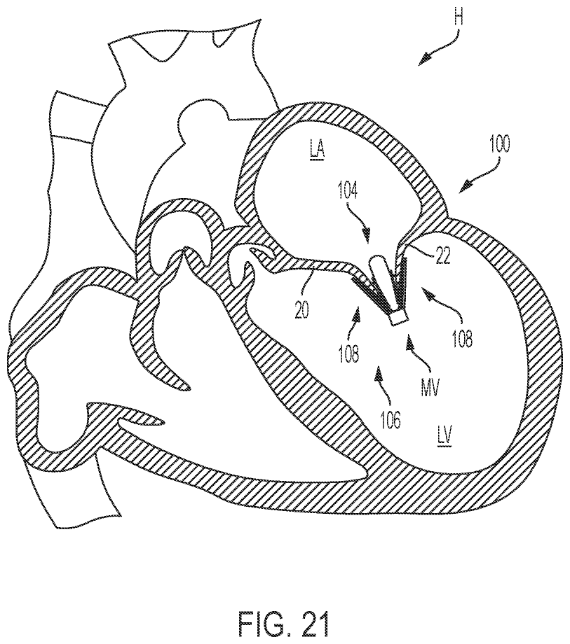

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), means for actuating or actuation element 112 and actuation lines 116 are then retracted and the device or implant 100 is fully closed and deployed in the native mitral valve MV.

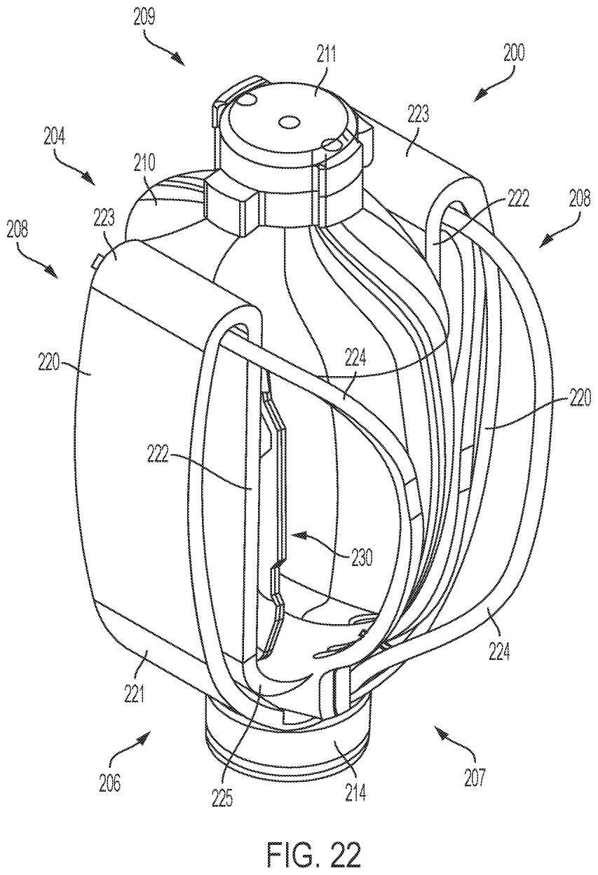

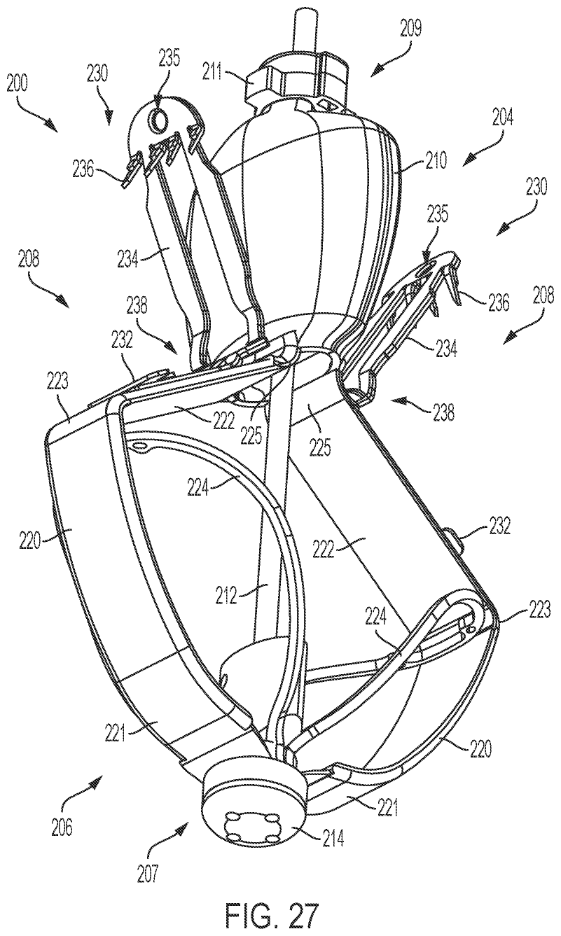

Referring now to FIGS. 22-27, an example of an implantable device or implant or implant 200 is shown. The implantable device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

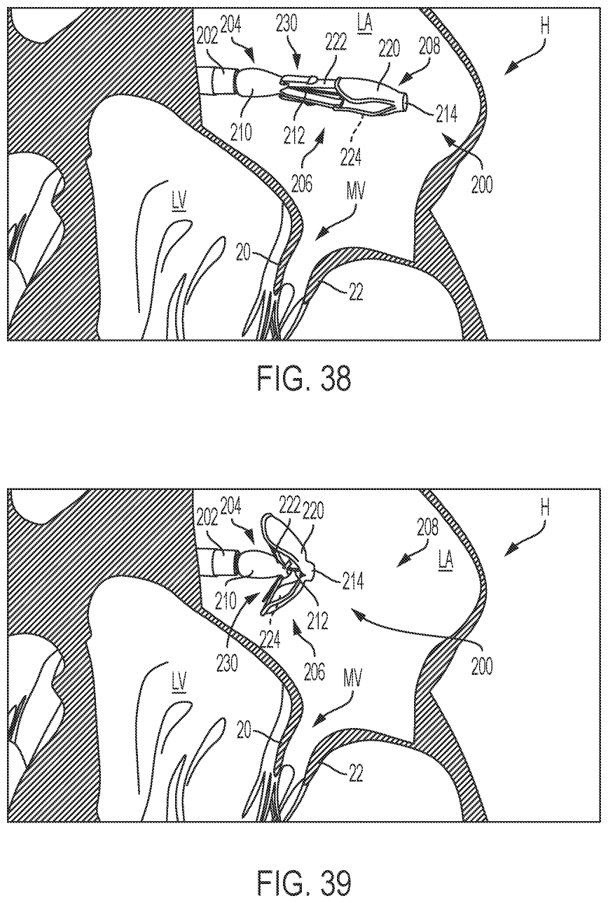

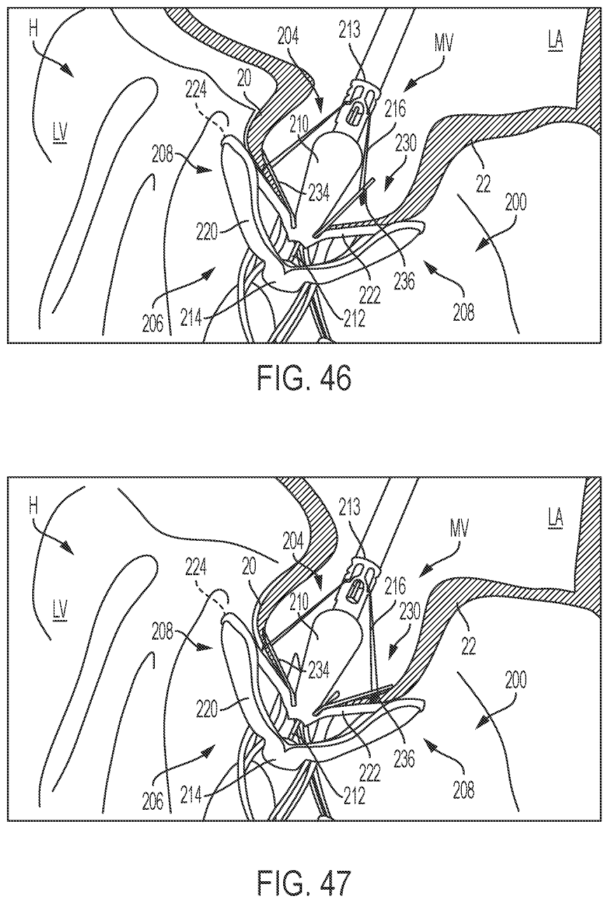

In some implementations, the implantable device or implant 200 includes a coaptation portion 204, a proximal or attachment portion 209, an anchor portion 206, and a distal portion 207. In some implementations, the coaptation portion 204 of the device optionally includes a coaptation element 210 (e.g., a spacer, coaption element, plug, membrane, sheet, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism 213 (FIGS. 43-49) of a delivery system 202 (FIGS. 38-42 and 49). Delivery system 202 can be the same as or similar to delivery system 102 described elsewhere and can comprise one or more of a catheter, a sheath, a guide catheter/sheath, a delivery catheter/sheath, a steerable catheter, an implant catheter, a tube, a channel, a pathway, combinations of these, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire-such as Nitinol—to provide shape-setting capability, or any other flexible material suitable for implantation in the human body.

An actuation element 212 (e.g., actuation shaft, actuation rod, actuation tube, actuation wire, actuation line, etc.) extends from the delivery system 202 to engage and enable actuation of the implantable device or implant 200. In some implementations, the actuation element 212 extends through the capture mechanism 213, proximal collar 211, and coaptation element 210 to engage a cap 214 of the distal portion 207. The actuation element 212 can be configured to removably engage the cap 214 with a threaded connection, or the like, so that the actuation element 212 can be disengaged and removed from the device 200 after implantation.

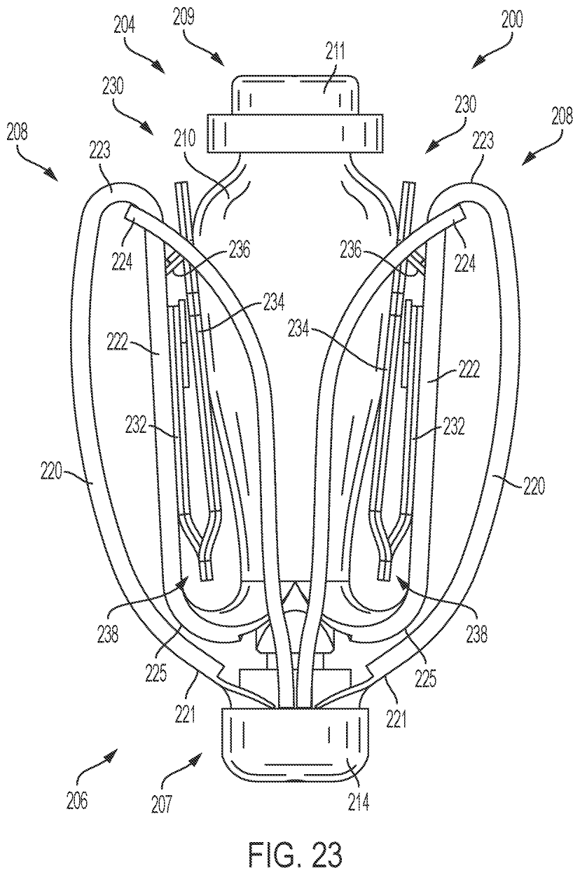

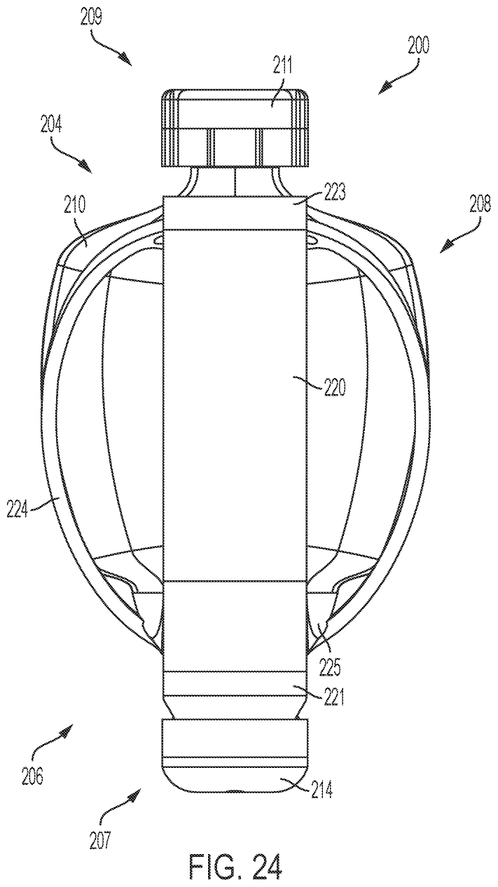

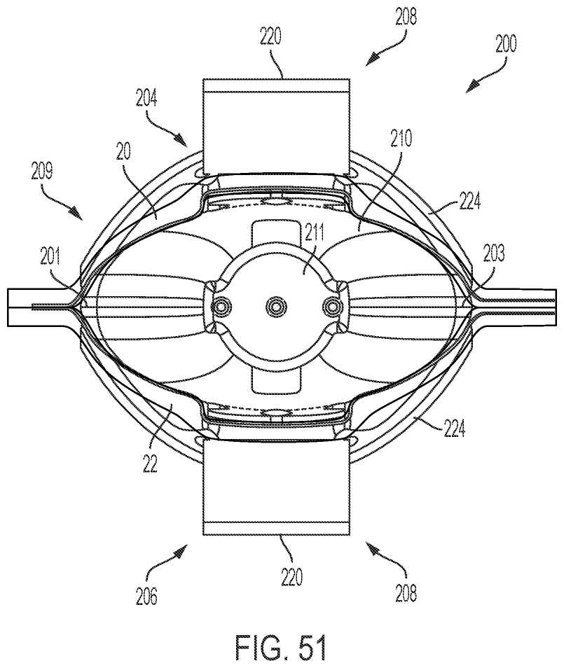

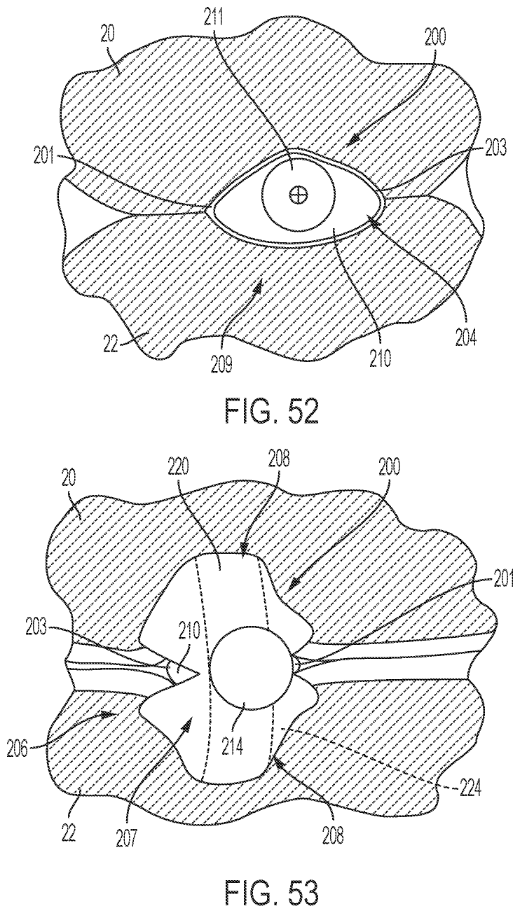

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above (e.g., FIG. 51) and has a tapered shape or cross-section when seen from a front view (e.g., FIG. 23) and a round shape or cross-section when seen from a side view (e.g., FIG. 24). A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion 232 of the clasps 230. The stiffening of the inner paddle allows the device to move to the various different positions shown and described herein. The inner paddle 222, the outer paddle 220, the coaptation can all be interconnected as described herein, such that the device 200 is constrained to the movements and positions shown and described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 provide additional pinching force between the inner paddles 222 and the coaptation element 210 and assist in wrapping the leaflets around the sides of the coaptation element 210 for a better seal between the coaptation element 210 and the leaflets, as can be seen in FIG. 51. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

Configuring the paddle frames 224 in this manner provides increased surface area compared to the outer paddles 220 alone. This can, for example, make it easier to grasp and secure the native leaflets. The increased surface area can also distribute the clamping force of the paddles 220 and paddle frames 224 against the native leaflets over a relatively larger surface of the native leaflets in order to further protect the native leaflet tissue. Referring again to FIG. 51, the increased surface area of the paddle frames 224 can also allow the native leaflets to be clamped to the implantable device or implant 200, such that the native leaflets coapt entirely around the coaptation member or coaptation element 210. This can, for example, improve sealing of the native leaflets 20, 22 and thus prevent or further reduce mitral regurgitation.

In some implementations the clasps comprise a moveable arm coupled to the anchors. In some implementations, the clasps 230 include a base or fixed arm 232, a moveable arm 234, optional barbs 236, and a joint portion 238. The fixed arms 232 are attached to the inner paddles 222, with the joint portion 238 disposed proximate the coaptation element 210. The joint portion 238 is spring-loaded so that the fixed and moveable arms 232, 234 are biased toward each other when the clasp 230 is in a closed condition. In some implementations, the clasps 230 include friction-enhancing elements or means for securing, such as barbs, protrusions, ridges, grooves, textured surfaces, adhesive, etc.

In some implementations, the fixed arms 232 are attached to the inner paddles 222 through holes or slots 231 with sutures (not shown). The fixed arms 232 can be attached to the inner paddles 222 with any suitable means, such as screws or other fasteners, crimped sleeves, mechanical latches or snaps, welding, adhesive, clamps, latches, or the like. The fixed arms 232 remain substantially stationary relative to the inner paddles 222 when the moveable arms 234 are opened to open the clasps 230 and expose the optional barbs or other friction-enhancing elements 236. The clasps 230 are opened by applying tension to actuation lines 216 (e.g., as shown in FIGS. 43-48) attached to holes 235 in the moveable arms 234, thereby causing the moveable arms 234 to articulate, pivot, and/or flex on the joint portions 238.

Referring now to FIG. 29, a close-up view of one of the leaflets 20, 22 grasped by a clasp such as clasp 230 is shown. The leaflet 20, 22 is grasped between the moveable and fixed arms 232, 234 of the clasp 230. The tissue of the leaflet 20, 22 is not pierced by the barbs or friction-enhancing elements 236, though in some implementations the barbs 236 can partially or fully pierce through the leaflet 20, 22. The angle and height of the barbs or friction-enhancing elements 236 relative to the moveable arm 234 helps to secure the leaflet 20, 22 within the clasp 230. In particular, a force pulling the implant off of the native leaflet 20, 22 will encourage the barbs or friction-enhancing elements 236 to further engage the tissue, thereby ensuring better retention. Retention of the leaflet 20, 22 in the clasp 230 is further improved by the position of fixed arm 232 near the barbs/friction-enhancing elements 236 when the clasp 230 is closed. In this arrangement, the tissue is formed by the fixed arms 232 and the moveable arms 234 and the barbs/friction-enhancing elements 236 into an S-shaped torturous path. Thus, forces pulling the leaflet 20, 22 away from the clasp 230 will encourage the tissue to further engage the barbs/friction-enhancing elements 236 before the leaflets 20, 22 can escape. For example, leaflet tension during diastole can encourage the barbs 236 to pull toward the end portion of the leaflet 20, 22. Thus, the S-shaped path can utilize the leaflet tension during diastole to more tightly engage the leaflets 20, 22 with the barbs/friction-enhancing elements 236.

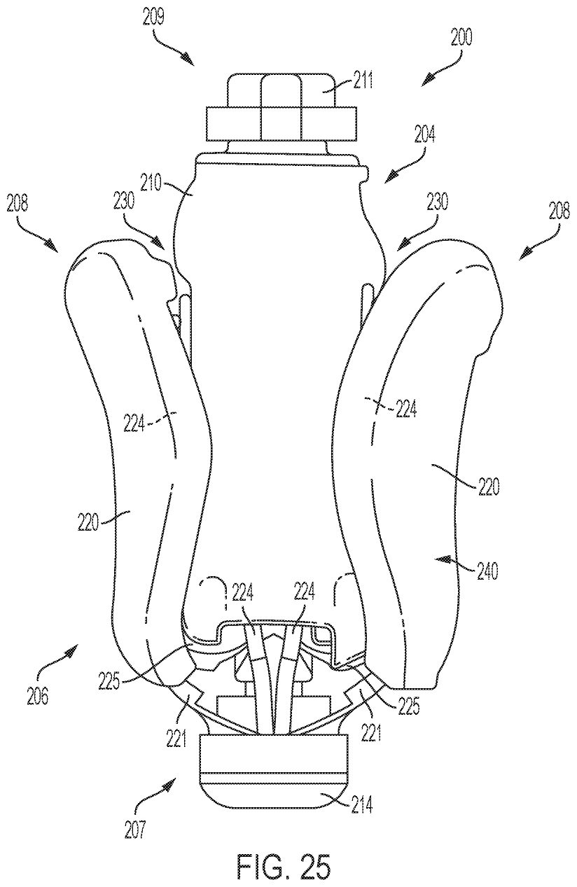

Referring to FIG. 25, the device or implant 200 can also include a cover 240. In some implementations, the cover 240 can be disposed on the coaptation element 210, the outer and inner paddles 220, 222, and/or the paddle frames 224. The cover 240 can be configured to prevent or reduce blood-flow through the device or implant 200 and/or to promote native tissue ingrowth. In some implementations, the cover 240 can be a cloth or fabric such as PET, velour, or other suitable fabric. In some implementations, in lieu of or in addition to a fabric, the cover 240 can include a coating (e.g., polymeric) that is applied to the implantable device or implant 200.

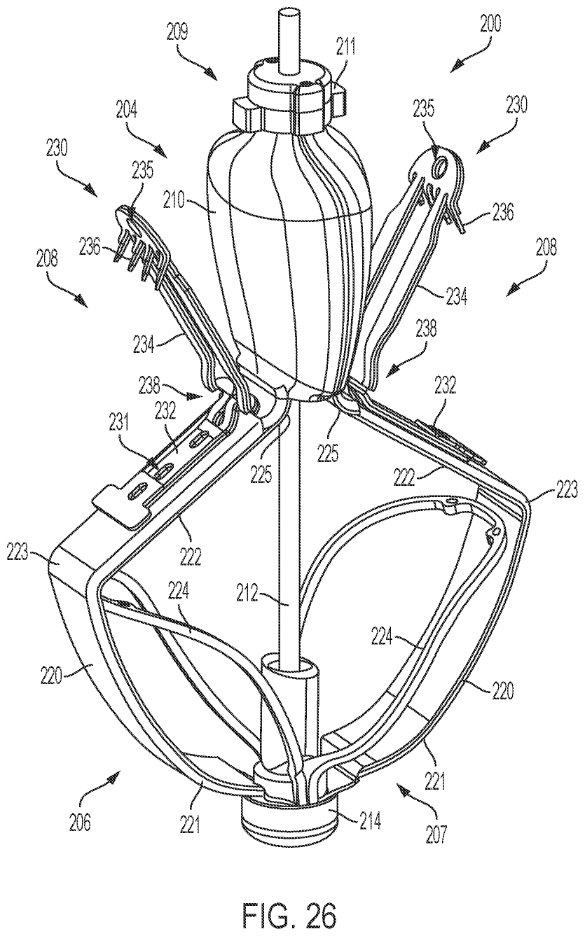

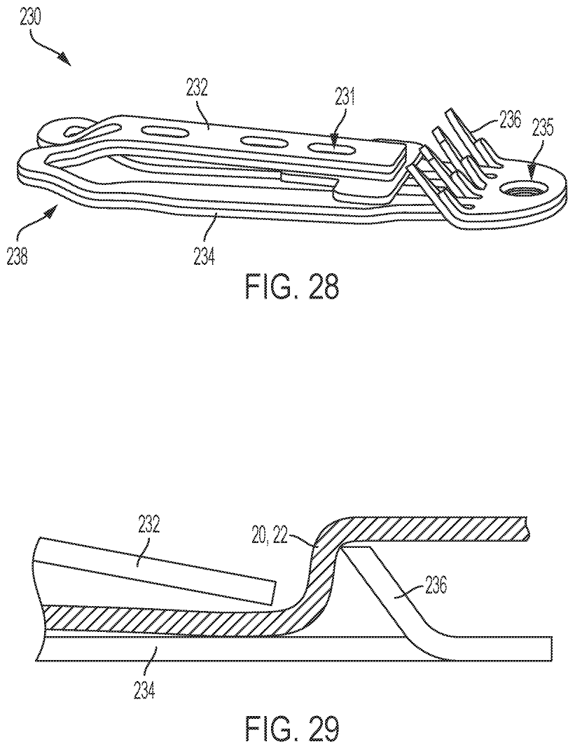

During implantation, the paddles 220, 222 of the anchors 208 are opened and closed to grasp the native valve leaflets 20, 22 between the paddles 220, 222 and the coaptation element 210. The anchors 208 are moved between a closed position (FIGS. 22-25) to various open positions (FIGS. 26-37) by extending and retracting the actuation element 212. Extending and retracting the actuation element 212 increases and decreases the spacing between the coaptation element 210 and the cap 214, respectively. The proximal collar 211 (or other attachment element) and the coaptation element 210 slide along the actuation element 212 during actuation so that changing of the spacing between the coaptation element 210 and the cap 214 causes the paddles 220, 220 to move between different positions to grasp the mitral valve leaflets 20, 22 during implantation.

As the device 200 is opened and closed, the pair of inner and outer paddles 222, 220 are moved in unison, rather than independently, by a single actuation element 212. Also, the positions of the clasps 230 are dependent on the positions of the paddles 222, 220. For example, the clasps 230 are arranged such that closure of the anchors 208 simultaneously closes the clasps 230. In some implementations, the device 200 can be made to have the paddles 220, 222 be independently controllable in the same manner (e.g., the device 100 illustrated in FIG. 15).

In some implementations, the clasps 230 further secure the native leaflets 20, 22 by engaging the leaflets 20, 22 with optional barbs and/or other friction-enhancing elements 236 and pinching the leaflets 20, 22 between the moveable and fixed arms 234, 232. In some implementations, the clasps 230 include barbs that increase friction with and/or can partially or completely puncture the leaflets 20, 22. The actuation lines 216 (FIGS. 43-48) can be actuated separately so that each clasp 230 can be opened and closed separately. Separate operation allows one leaflet 20, 22 to be grasped at a time, or for the repositioning of a clasp 230 on a leaflet 20, 22 that was insufficiently grasped, without altering a successful grasp on the other leaflet 20, 22. The clasps 230 can be fully opened and closed when the inner paddle 222 is not closed, thereby allowing leaflets 20, 22 to be grasped in a variety of positions as the particular situation requires.

Referring now to FIGS. 22-25, the device 200 is shown in a closed position. When closed, the inner paddles 222 are disposed between the outer paddles 220 and the coaptation element 210. The clasps 230 are disposed between the inner paddles 222 and the coaptation element 210. Upon successful capture of native leaflets 20, 22 the device 200 is moved to and retained in the closed position so that the leaflets 20, 22 are secured within the device 200 by the clasps 230 and are pressed against the coaptation element 210 by the paddles 220, 222. The outer paddles 220 can have a wide curved shape that fits around the curved shape of the coaptation element 210 to grip the leaflets 20, 22 more securely when the device 200 is closed (e.g., as can be seen in FIG. 51). The curved shape and rounded edges of the outer paddle 220 also prohibits or inhibits tearing of the leaflet tissue.

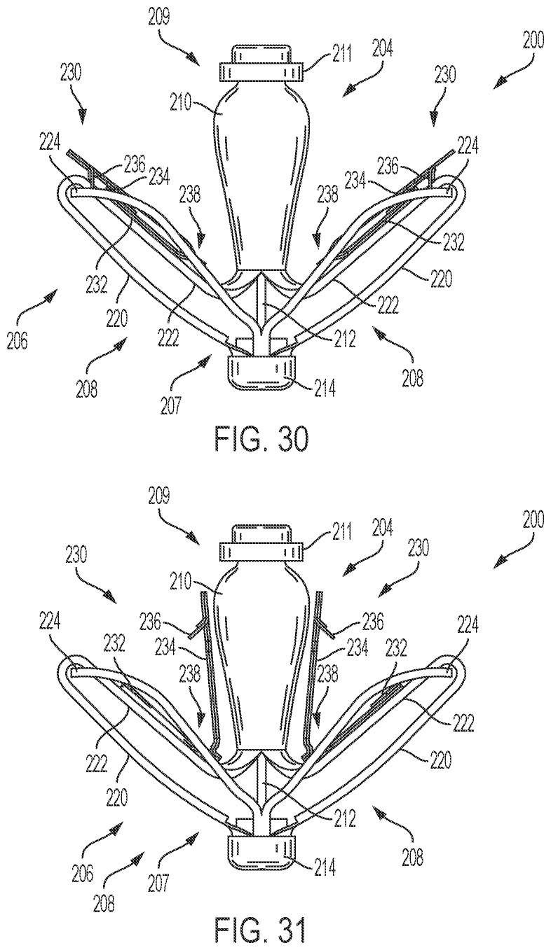

Referring now to FIGS. 30-37, the implantable device or implant 200 described above is shown in various positions and configurations ranging from partially open to fully open. The paddles 220, 222 of the device 200 transition between each of the positions shown in FIGS. 30-37 from the closed position shown in FIGS. 22-25 up extension of the actuation element 212 from a fully retracted to fully extended position.

Referring now to FIGS. 30-31, the device 200 is shown in a partially open position or capture-ready position. The device 200 is moved into the partially open position by extending the actuation element 212. Extending the actuation element 212 pulls down on the bottom portions of the outer paddles 220 and paddle frames 224. The outer paddles 220 and paddle frames 224 pull down on the inner paddles 222, where the inner paddles 222 are connected to the outer paddles 220 and the paddle frames 224. Because the proximal collar 211 (or other attachment element) and coaptation element 210 are held in place by the capture mechanism 213, the inner paddles 222 are caused to articulate, pivot, and/or flex in an opening direction. The inner paddles 222, the outer paddles 220, and the paddle frames all flex to the position shown in FIGS. 30-31. Opening the paddles 222, 220 and frames 224 forms a gap between the coaptation element 210 and the inner paddle 222 that can receive and grasp the native leaflets 20, 22. This movement also exposes the clasps 230 that can be moved between closed (FIG. 30) and open (FIG. 31) positions to form a second gap for grasping the native leaflets 20, 22. The extent of the gap between the fixed and moveable arms 232, 234 of the clasp 230 is limited to the extent that the inner paddle 222 has spread away from the coaptation element 210.

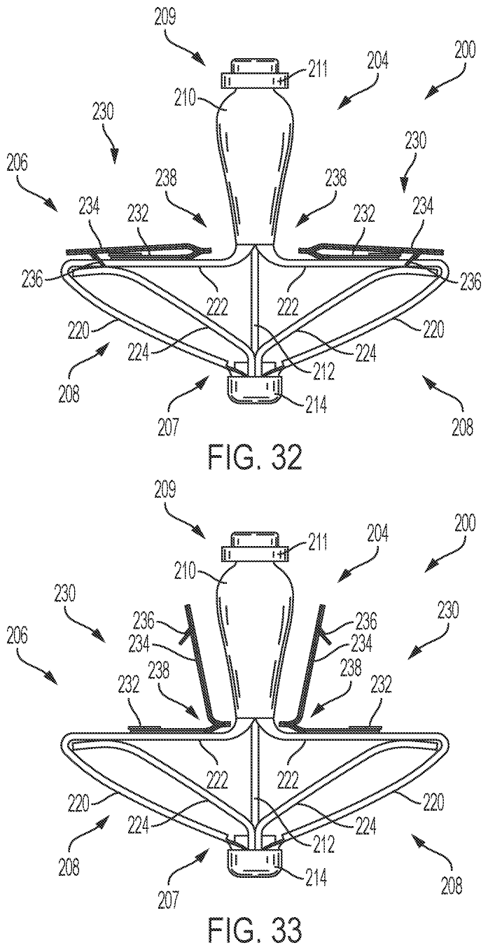

Referring now to FIGS. 32-33, the device 200 is shown in a laterally extended or open position. The device 200 is moved into the laterally extended or open position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the laterally extended or open position, the inner paddles 222 extend horizontally more than in other positions of the device 200 and form an approximately 90-degree angle with the coaptation element 210. Similarly, the paddle frames 224 are at their maximum spread position when the device 200 is in the laterally extended or open position. The increased gap between the coaptation element 210 and inner paddle 222 formed in the laterally extended or open position allows clasps 230 to open further (FIG. 33) before engaging the coaptation element 210, thereby increasing the size of the gap between the fixed and moveable arms 232, 234.

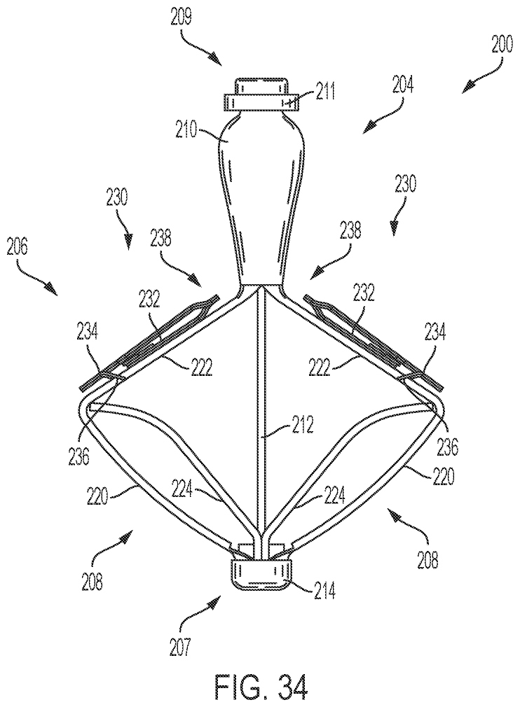

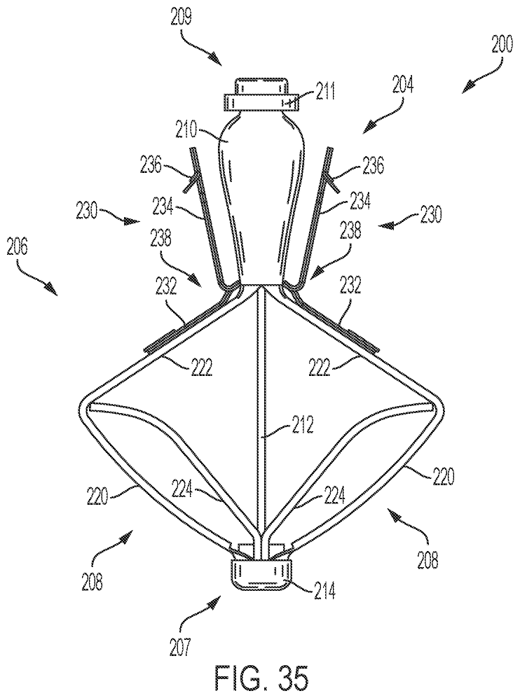

Referring now to FIGS. 34-35, the example device 200 is shown in a three-quarters extended position. The device 200 is moved into the three-quarters extended position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the three-quarters extended position, the inner paddles 222 are open beyond 90 degrees to an approximately 135-degree angle with the coaptation element 210. The paddle frames 224 are less spread than in the laterally extended or open position and begin to move inward toward the actuation element 212 as the actuation element 212 extends further. The outer paddles 220 also flex back toward the actuation element 212. As with the laterally extended or open position, the increased gap between the coaptation element 210 and inner paddle 222 formed in the laterally extended or open position allows clasps 230 to open even further (FIG. 35), thereby increasing the size of the gap between the fixed and moveable arms 232, 234.

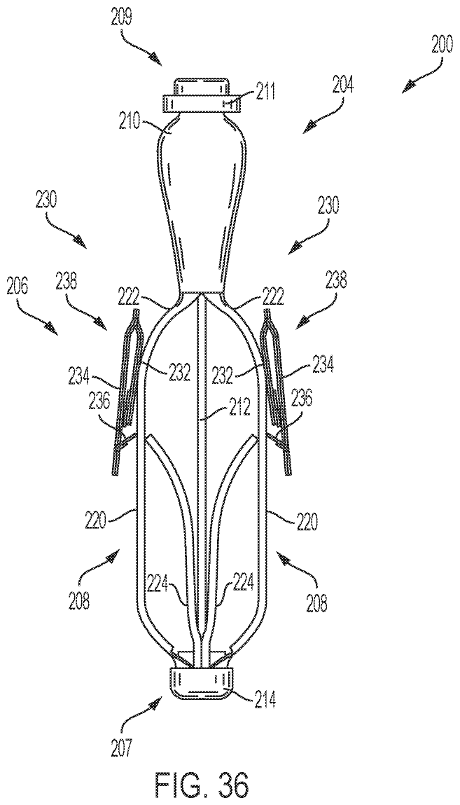

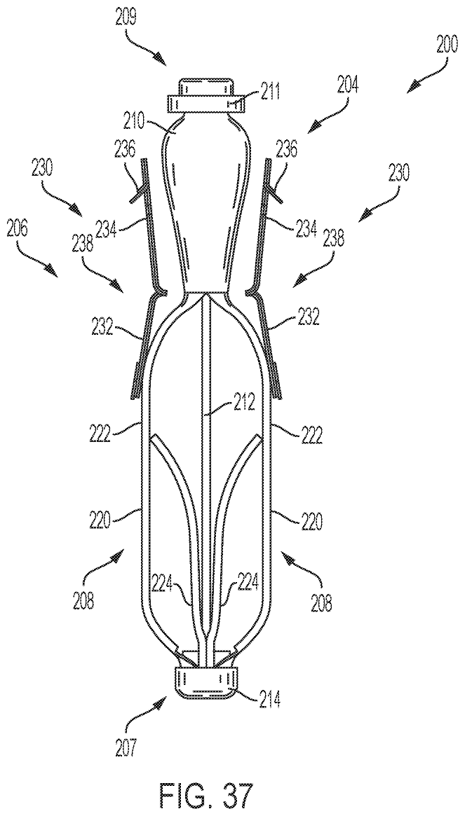

Referring now to FIGS. 36-37, the example device 200 is shown in a fully extended position. The device 200 is moved into the fully extended position by continuing to extend the actuation element 212 described above, thereby increasing the distance between the coaptation element 210 and the cap 214 of the distal portion 207 to a maximum distance allowable by the device 200. Continuing to extend the actuation element 212 pulls down on the outer paddles 220 and paddle frames 224, thereby causing the inner paddles 222 to spread apart further from the coaptation element 210. The outer paddles 220 and paddle frames 224 move to a position where they are close to the actuation element. In the fully extended position, the inner paddles 222 are open to an approximately 180-degree angle with the coaptation element 210. The inner and outer paddles 222, 220 are stretched straight in the fully extended position to form an approximately 180-degree angle between the paddles 222, 220. The fully extended position of the device 200 provides the maximum size of the gap between the coaptation element 210 and inner paddle 222, and, in some implementations, allows clasps 230 to also open fully to approximately 180 degrees (FIG. 37) between the fixed and moveable arms 232, 234 of the clasp 230. The position of the device 200 is the longest and the narrowest configuration. Thus, the fully extended position of the device 200 can be a desirable position for bailout of the device 200 from an attempted implantation or can be a desired position for placement of the device in a delivery catheter, or the like.

Configuring the device or implant 200 such that the anchors 208 can extend to a straight or approximately straight configuration (e.g., approximately 120-180 degrees relative to the coaptation element 210) can provide several advantages. For example, this configuration can reduce the radial crimp profile of the device or implant 200. It can also make it easier to grasp the native leaflets 20, 22 by providing a larger opening between the coaptation element 210 and the inner paddles 222 in which to grasp the native leaflets 20, 22. Additionally, the relatively narrow, straight configuration can prevent or reduce the likelihood that the device or implant 200 will become entangled in native anatomy (e.g., chordae tendineae CT shown in FIGS. 3 and 4) when positioning and/or retrieving the device or implant 200 into the delivery system 202.

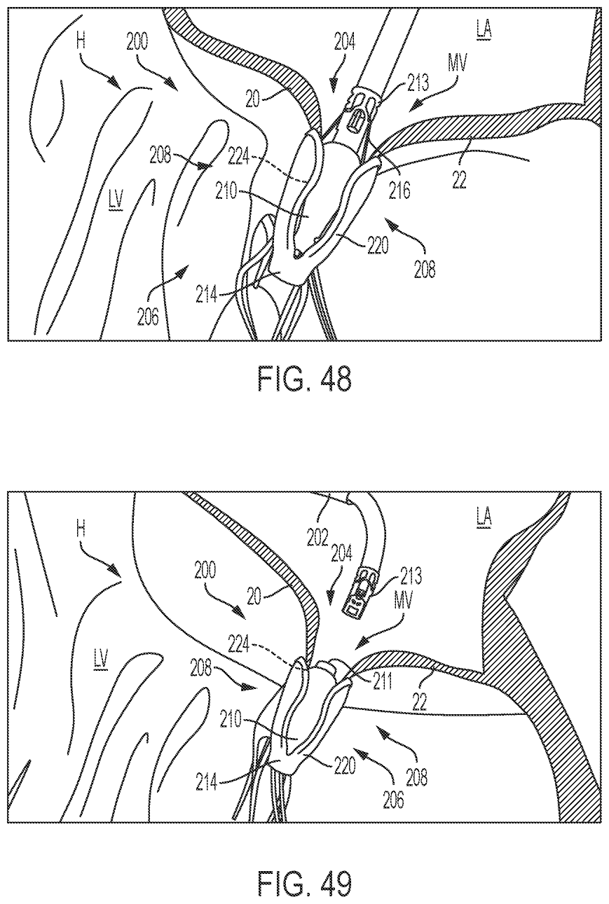

Referring now to FIGS. 38-49, an example implantable device 200 is shown being delivered and implanted within the native mitral valve MV of the heart H. As described above, the device 200 shown in FIGS. 38-49 includes the optional covering 240 (e.g., FIG. 25) over the coaptation element 210, clasps 230, inner paddles 222 and/or the outer paddles 220. The device 200 is deployed from a delivery system 202 (e.g., which can comprise an implant catheter that is extendable from a steerable catheter and/or a guide sheath) and is retained by a capture mechanism 213 (see e.g., FIGS. 43 and 48) and is actuated by extending or retracting the actuation element 212. Fingers of the capture mechanism 213 removably attach the collar 211 to the delivery system 202. In some implementations, the capture mechanism 213 is held closed around the collar 211 by the actuation element 212, such that removal of the actuation element 212 allows the fingers of the capture mechanism 213 to open and release the collar 211 to decouple the capture mechanism 213 from the device 200 after the device 200 has been successfully implanted.

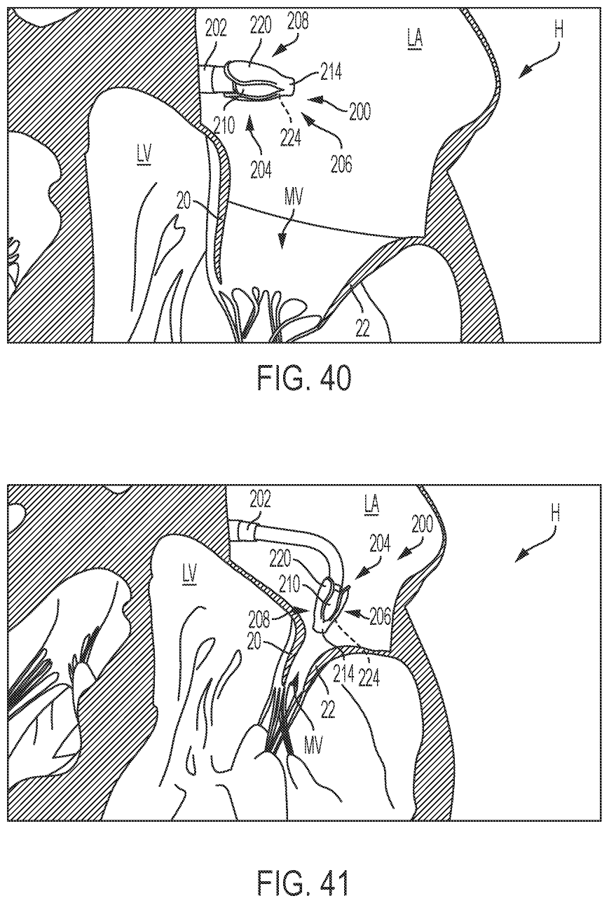

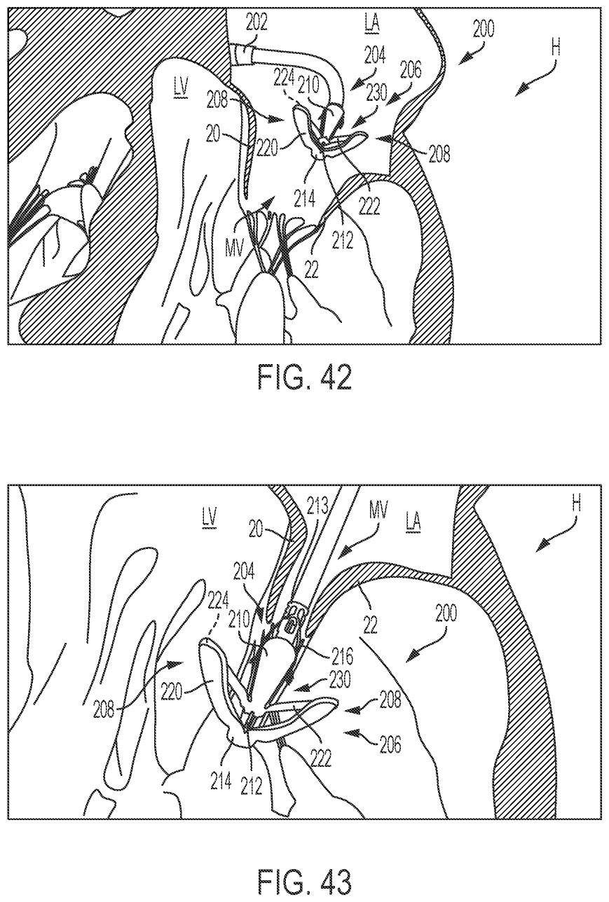

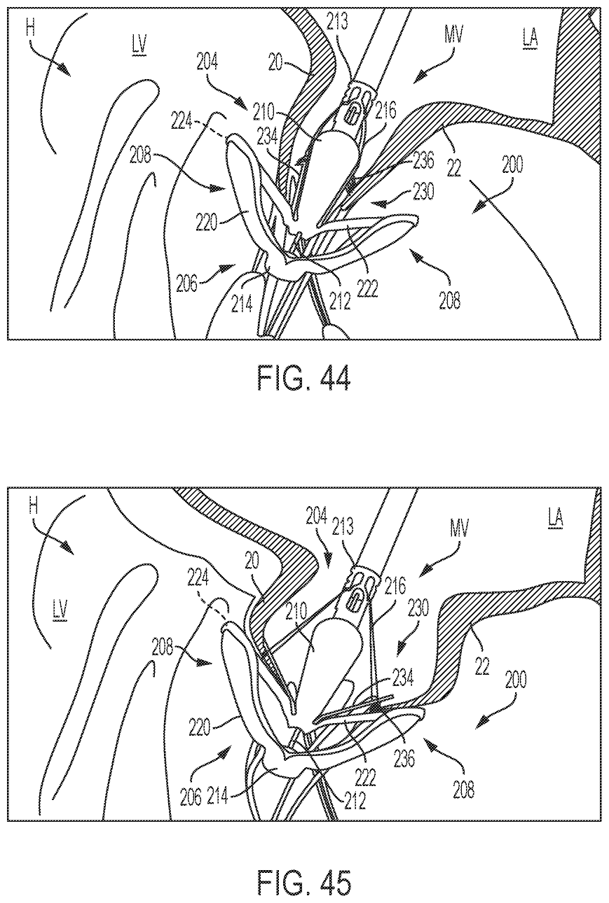

Referring now to FIG. 38, the delivery system 202 (e.g., a delivery catheter/sheath thereof) is inserted into the left atrium LA through the septum and the device/implant 200 is deployed from the delivery system 202 (e.g., an implant catheter retaining the device/implant can be extended to deploy the device/implant out from a steerable catheter) in the fully open condition for the reasons discussed above with respect to the device 100. The actuation element 212 is then retracted to move the device 200 through the partially closed condition (FIG. 39) and to the fully closed condition shown in FIGS. 40-41. Then the delivery system or catheter maneuvers the device/implant 200 towards the mitral valve MV as shown in FIG. 41. Referring now to FIG. 42, when the device 200 is aligned with the mitral valve MV, the actuation element 212 is extended to open the paddles 220, 222 into the partially opened position and the actuation lines 216 (FIGS. 43-48) are retracted to open the clasps 230 to prepare for leaflet grasp. Next, as shown in FIGS. 43-44, the partially open device 200 is inserted through the native valve (e.g., by advancing an implant catheter from a steerable catheter) until leaflets 20, 22 are properly positioned in between the inner paddles 222 and the coaptation element 210 and inside the open clasps 230.

FIG. 45 shows the device 200 with both clasps 230 closed, though the optional barbs 236 of one clasp 230 missed one leaflet 22. As can be seen in FIGS. 45-47, the out of position clasp 230 is opened and closed again to properly grasp the missed leaflet 22. When both leaflets 20, 22 are grasped properly, the actuation element 212 is retracted to move the device 200 into the fully closed position shown in FIG. 48. With the device 200 fully closed and implanted in the native valve, the actuation element 212 is disengaged from the cap 214 and is withdrawn to release the capture mechanism 213 from the proximal collar 211 (or other attachment element) so that the capture mechanism 213 can be withdrawn into the delivery system 202 (e.g., into a catheter/sheath), as shown in FIG. 49. Once deployed, the device 200 can be maintained in the fully closed position with a mechanical means such as a latch or can be biased to remain closed through the use of spring material, such as steel, and/or shape-memory alloys such as Nitinol. For example, the paddles 220, 222 can be formed of steel or Nitinol shape-memory alloy produced in a wire, sheet, tubing, or laser sintered powder—and are biased to hold the outer paddles 220 closed around the inner paddles 222, coaptation element 210, and/or the clasps 230 pinched around native leaflets 20, 22.

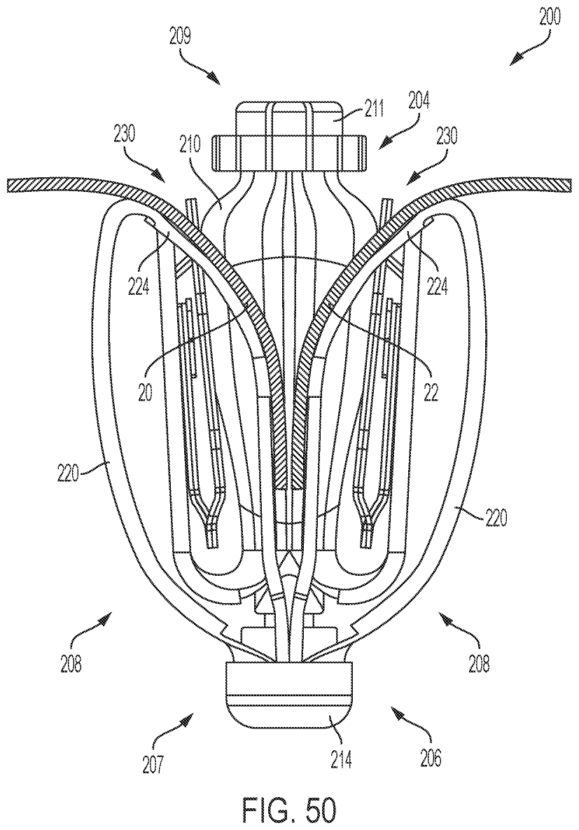

Referring to FIGS. 50-54, once the device 200 is implanted in a native valve, the coaptation element 210 functions as a gap filler in the valve regurgitant orifice, such as the gap 26 in the mitral valve MV illustrated by FIG. 6 or a gap in another native valve. In some implementations, when the device 200 has been deployed between the two opposing valve leaflets 20, 22, the leaflets 20, 22 no longer coapt against each other in the area of the coaptation element 210, but instead coapt against the coaptation element 210. This reduces the distance the leaflets 20, 22 need to be approximated to close the mitral valve MV during systole, thereby facilitating repair of functional valve disease that may be causing mitral regurgitation. A reduction in leaflet approximation distance can result in several other advantages as well. For example, the reduced approximation distance required of the leaflets 20, 22 reduces or minimizes the stress experienced by the native valve. Shorter approximation distance of the valve leaflets 20,22 can also require less approximation forces which can result in less tension experienced by the leaflets 20, 22 and less diameter reduction of the valve annulus. The smaller reduction of the valve annulus—or none at all—can result in less reduction in valve orifice area as compared to a device without a coaptation element or spacer. In this way, the coaptation element 210 can reduce the transvalvular gradients.

To adequately fill the gap 26 between the leaflets 20, 22, the device 200 and the components thereof can have a wide variety of different shapes and sizes. For example, the outer paddles 220 and paddle frames 224 can be configured to conform to the shape or geometry of the coaptation element 210 as is shown in FIGS. 50-54. As a result, the outer paddles 220 and paddle frames 224 can mate with both the coaptation element 210 and the native valve leaflets 20, 22. In some implementations, when the leaflets 20, 22 are coapted against the coaptation element 210, the leaflets 20, 22 fully surround or “hug” the coaptation element 210 in its entirety, thus small leaks at lateral and medial aspects 201, 203 of the coaptation element 210 can be prevented or reduced. The interaction of the leaflets 20, 22 and the device 200 is made clear in FIG. 51, which shows a schematic atrial or surgeon's view that shows the paddle frame 224 (which would not actually be visible from a true atrial view, e.g., FIG. 52), conforming to the coaptation element 210 geometry. The opposing leaflets 20, 22 (the ends of which would also not be visible in the true atrial view, e.g., FIG. 52) being approximated by the paddle frames 224, to fully surround or “hug” the coaptation element 210.

This coaptation of the leaflets 20, 22 against the lateral and medial aspects 201, 203 of the coaptation element 210 (shown from the atrial side in FIG. 52, and the ventricular side in FIG. 53) would seem to contradict the statement above that the presence of a coaptation element 210 minimizes the distance the leaflets need to be approximated. However, the distance the leaflets 20, 22 need to be approximated is still minimized if the coaptation element 210 is placed precisely at a regurgitant gap 26 and the regurgitant gap 26 is less than the width (medial-lateral) of the coaptation element 210.

FIG. 50 illustrates the geometry of the coaptation element 210 and the paddle frame 224 from an LVOT perspective. As can be seen in this view, the coaptation element 210 has a tapered shape being smaller in dimension in the area closer to where the inside surfaces of the leaflets 20, 22 are required to coapt and increase in dimension as the coaptation element 210 extends toward the atrium. Thus, the depicted native valve geometry is accommodated by a tapered coaptation element geometry. Still referring to FIG. 50, the tapered coaptation element geometry, in conjunction with the illustrated expanding paddle frame 224 shape (toward the valve annulus) can help to achieve coaptation on the lower end of the leaflets, reduce stress, and minimize transvalvular gradients.

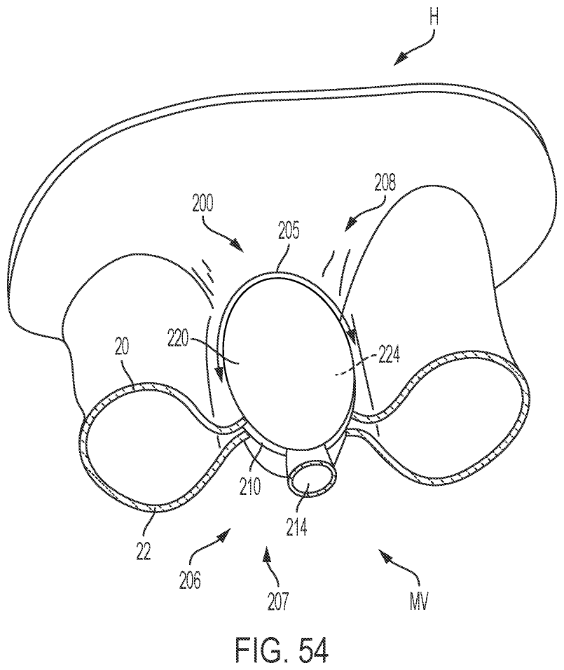

Referring to FIG. 54, the shape of the coaptation element 210 and the paddle frames 224 can be defined based on an Intra-Commissural view of the native valve and the device 200. Two factors of these shapes are leaflet coaptation against the coaptation element 210 and reduction of stress on the leaflets due to the coaptation. Referring to FIGS. 54 and 24, to both coapt the valve leaflets 20, 22 against the coaptation element 210 and reduce the stress applied to the valve leaflets 20, 22 by the coaptation element 210 and/or the paddle frames 224, the coaptation element 210 can have a round or rounded shape and the paddle frames 224 can have a full radius that spans nearly the entirety of the paddle frame 224. The round shape of the coaptation element 210 and/or the illustrated fully rounded shape of the paddle frames 224 distributes the stresses on the leaflets 20, 22 across a large, curved engagement area 205. For example, in FIG. 54, the force on the leaflets 20, 22 by the paddle frames is spread along the entire rounded length of the paddle frame 224, as the leaflets 20 try to open during the diastole cycle.

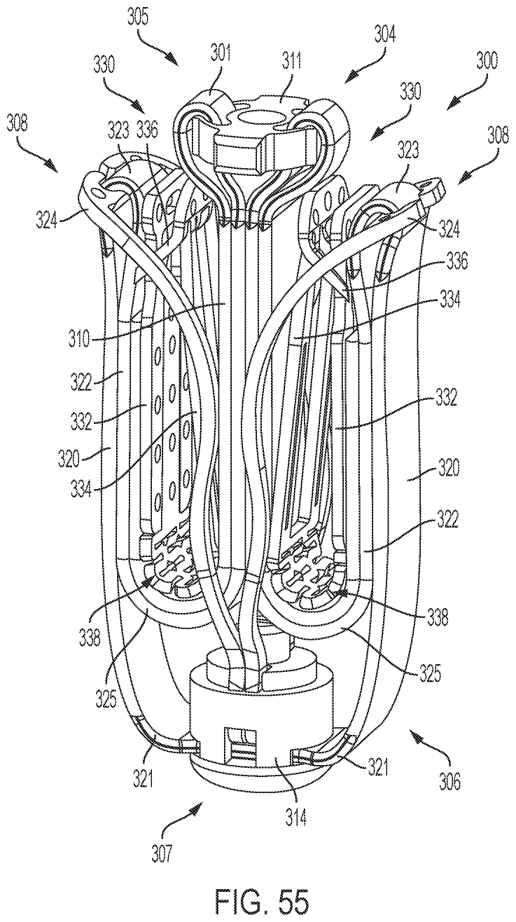

Referring now to FIG. 55, an example of an implantable device or implant 300 is shown. The implantable device 300 is one of the many different configurations that the device 100 that is schematically illustrated in FIGS. 8-14 can take. The device 300 can include any other features for an implantable device or implant discussed in the present application, and the device 300 can be positioned to engage valve tissue 20, 22 as part of any suitable valve repair system (e.g., any valve repair system disclosed in the present application).

The implantable device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism (e.g., a capture mechanism such as the capture mechanism 213 shown in FIGS. 43-49) of a delivery system (e.g., a delivery system such as the system shown in FIGS. 38-42 and 49).

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation member or coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation member or coaptation element 310, the coaptation member or coaptation element 310 and the anchors 308 can be coupled together in various ways. For example, as shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from the continuous strip of fabric 301.

Like the anchors 208 of the implantable device or implant 200 described above, the anchors 308 can be configured to move between various configurations by axially moving the distal end of the device (e.g., cap 314, etc.) relative to the proximal end of the device (e.g., proximal collar 311 or other attachment element, etc.) and thus the anchors 308 move relative to a midpoint of the device. This movement can be along a longitudinal axis extending between the distal end (e.g., cap 314, etc.) and the proximal end (e.g., collar 311 or other attachment element, etc.) of the device. For example, the anchors 308 can be positioned in a fully extended, fully elongated, or straight configuration (e.g., similar to the configuration of device 200 shown in FIG. 36) by moving the distal end (e.g., cap 314, etc.) away from the proximal end of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the coaptation element 310 (e.g., similar to the configuration of device 200 shown in FIG. 36). From the fully elongated or straight configuration, the anchors 308 can be moved to a fully folded or closed configuration (e.g., FIG. 55), e.g., by moving the proximal end and distal end toward each other and/or toward a midpoint or center of the device. Initially, as the distal end (e.g., cap 314, etc.) moves toward the proximal end and/or midpoint or center of the device, the anchors 308 bend at connection portions 321, 323, 325, and the connection portions 323 move radially outwardly relative to the longitudinal axis of the device 300 and axially toward the midpoint and/or toward the proximal end of the device (e.g., similar to the configuration of device 200 shown in FIG. 34). As the cap 314 continues to move toward the midpoint and/or toward the proximal end of the device, the connection portions 323 move radially inwardly relative to the longitudinal axis of the device 300 and axially toward the proximal end of the device (e.g., similar to the configuration of device 200 shown in FIG. 30).

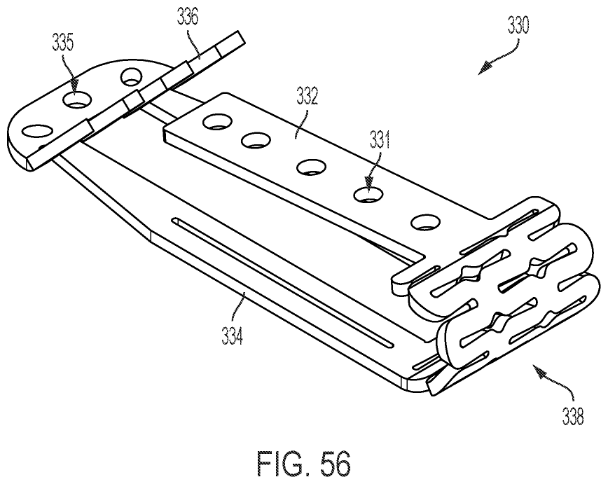

In some implementations, the clasps comprise a moveable arm coupled to an anchor. In some implementations, the clasps 330 (as shown in detail in FIG. 56) include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots 331 with sutures (not shown). The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336. The clasps 330 are opened by applying tension to actuation lines (e.g., the actuation lines 216 shown in FIGS. 43-48) attached to holes 335 in the moveable arms 334, thereby causing the moveable arms 334 to articulate, pivot, and/or flex on the joint portions 338.

In short, the implantable device or implant 300 is similar in configuration and operation to the implantable device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 55 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 55, the strip of material 301 begins and ends in the location of the inner paddles 322.

As with the implantable device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

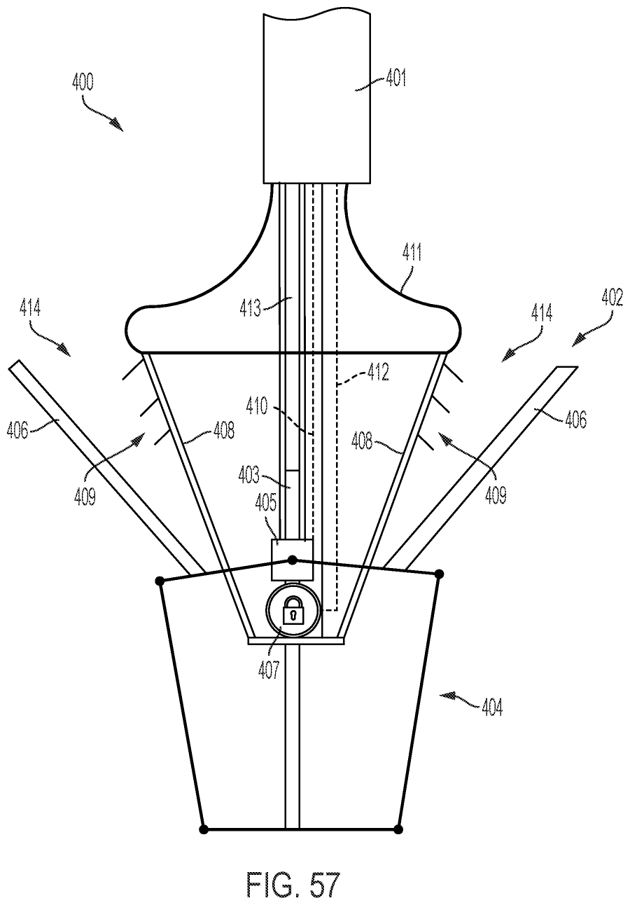

The concepts disclosed by the present application can be used with a wide variety of different valve treatment devices. FIGS. 57-63 illustrate one example valve treatment system configured as a valve repair system 400 for repairing a native valve of a patient to which the concepts of the present application can be applied. Though the concepts of the present application can be applied to many more valve treatment systems (e.g., many other valve repair systems and many valve replacement systems).

The valve repair system 400 includes a delivery system or delivery device 401 and an implant 402 configured as a valve repair device. The implant or valve repair device 402 includes a base assembly 404, a pair of paddles 406, and a pair of gripping members 408. In some implementations, the paddles 406 can be integrally formed with the base assembly. For example, the paddles 406 can be formed as extensions of links of the base assembly. In the illustrated example, the base assembly 404 of the valve repair device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include an optional barbed portion 409 for attaching the gripping members to valve tissue when the implant or valve repair device 402 is attached to the valve tissue. The gripping member 408 forms a means for gripping the valve tissue (in particular tissue of the valve leaflets) with a sticking means or portion such as the barbed portion 409. When the paddles 406 are in the closed position, the paddles engage the gripping members 408, such that, when valve tissue is attached to the barbed portion 409 of the gripping members, the paddles act as holding or securing means to hold the valve tissue at the gripping members and to secure the implant 402 to the valve tissue. In some implementations, the gripping members 408 are configured to engage the paddles 406 such that the barbed portion 409 engages the valve tissue member and the paddles 406 to secure the implant 402 to the valve tissue member. For example, in certain situations, it can be advantageous to have the paddles 406 maintain an open position and have the gripping members 408 move outward toward the paddles 406 to engage valve tissue and the paddles 406.

While the examples shown in FIGS. 57-63 illustrate a pair of paddles 406 and a pair of gripping members 408, it should be understood that the implant or valve repair device 402 can include any suitable number of paddles and gripping members.

In some implementations, the valve repair system 400 includes a placement shaft 413 that is removably attached to the shaft 403 of the base assembly 404 of the valve repair device 402. After the valve repair device 402 is secured to valve tissue, the placement shaft 413 is removed from the shaft 403 to remove the valve repair device 402 from the remainder of the valve repair system 400, such that the valve repair device 402 can remain attached to the valve tissue, and the delivery device 401 can be removed from a patient's body.

The valve repair system 400 can also include a paddle control mechanism 410, a gripper control mechanism 411, and a lock control mechanism 412. The paddle control mechanism 410 is mechanically attached to the coupler 405 to move the coupler along the shaft, which causes the paddles 406 to move between the open and closed positions. The paddle control mechanism 410 can take any suitable form, such as, for example, a shaft or rod. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture or wire, a rod, a catheter, etc.

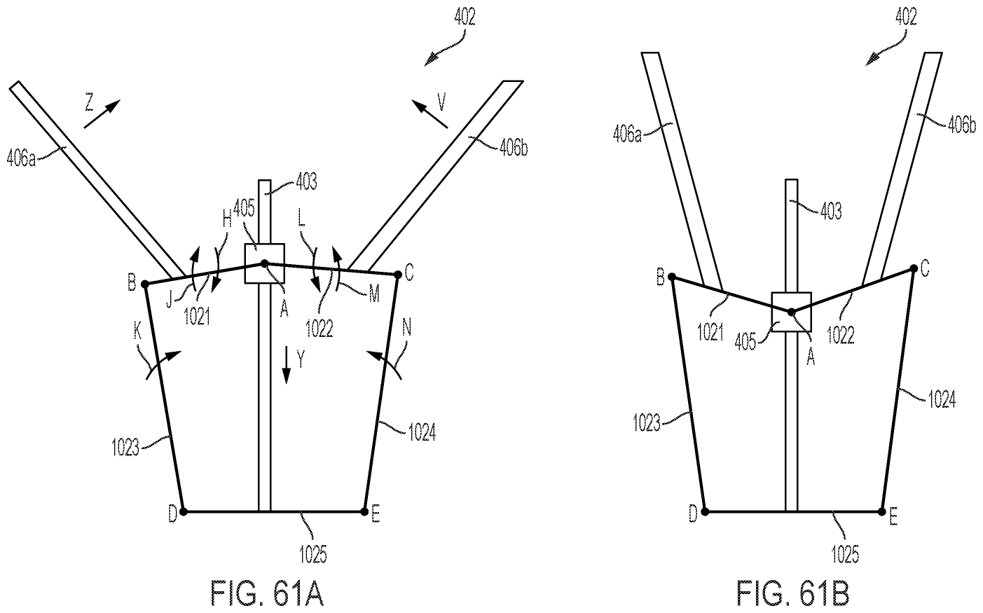

The lock control mechanism 412 is configured to lock and unlock the lock. The lock 407 serves as a locking means for locking the coupler 405 in a stationary position with respect to the shaft 403 and can take a wide variety of different forms and the type of lock control mechanism 412 may be dictated by the type of lock used. In some implementations, the lock 407 takes the form of locks often used in caulk guns. That is, the lock 407 includes a pivotable plate having a hole, in which the shaft 403 of the valve repair device 402 is disposed within the hole of the pivotable plate. In this example, when the pivotable plate is in the tilted position, the pivotable plate engages the shaft 403 to maintain a position on the shaft 403, but, when the pivotable plate is in a substantially non-tilted position, the pivotable plate can be moved along the shaft (which allows the coupler 405 to move along the shaft 403). In other words, the coupler 405 is prevented from moving in the direction Y (as shown in FIG. 61A) along the shaft 403 when the pivotable plate of the lock 407 is in a tilted (or locked) position, and the coupler is allowed to move in the direction Y along the shaft 403 when the pivotable plate is in a substantially non-tilted (or unlocked) position. In some implementations in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position. In some implementations, the pivotable plate of the lock 407 is biased in the tilted (or locked) position, and the lock control mechanism 412 is used to move the plate from the tilted position to the substantially non-tilted (or unlocked) position. In some implementations, the pivotable plate of the lock 407 is biased in the substantially non-tilted (or unlocked) position, and the lock control mechanism 412 is used to move the plate from the substantially non-tilted position to the tilted (or locked) position.

FIGS. 61A-61B illustrate the valve repair device 402 moving from an open position (as shown in FIG. 61A) to a closed position (as shown in FIG. 61). The base assembly 404 includes a first link 1021 extending from point A to point B, a second link 1022 extending from point A to point C, a third link 1023 extending from point B to point D, a fourth link 1024 extending from point C to point E, and a fifth link 1025 extending from point D to point E. The coupler 405 is movably attached to the shaft 403, and the shaft 403 is fixed to the fifth link 1025. The first link 1021 and the second link 1022 are pivotally attached to the coupler 405 at point A, such that movement of the coupler 405 along the shaft 403 moves the location of point A and, consequently, moves the first link 1021 and the second link 1022. The first link 1021 and the third link 1023 are pivotally attached to each other at point B, and the second link 1022 and the fourth link 1024 are pivotally attached to each other at point C. One paddle 406a is attached to first link 1021 such that movement of first link 1021 causes the paddle 406a to move, and the other paddle 406b is attached to the second link 1022 such that movement of the second link 1022 causes the paddle 406b to move. In some implementations, the paddles 406a, 406b can be connected to links 1023, 1024 or be extensions of links 1023, 1024.

In order to move the valve repair device from the open position (as shown in FIG. 61A) to the closed position (as shown in FIG. 61), the coupler 405 is moved along the shaft 403 in the direction Y, which moves the pivot point A for the first links 1021 and the second link 1022 to a new position. Movement of the coupler 405 (and pivot point A) in the direction Y causes a portion of the first link 1021 near point A to move in the direction H, and the portion of the first link 1021 near point B to move in the direction J. The paddle 406a is attached to the first link 1021 such that movement of the coupler 405 in the direction Y causes the paddle 406a to move in the direction Z. In addition, the third link 1023 is pivotally attached to the first link 1021 at point B such that movement of the coupler 405 in the direction Y causes the third link 1023 to move in the direction K. Similarly, movement of the coupler 405 (and pivot point A) in the direction Y causes a portion of the second link 1022 near point A to move in the direction L, and the portion of the second link 1022 near point C to move in the direction M. The paddle 406b is attached to the second link 1022 such that movement of the coupler 405 in the direction Y causes the paddle 406b to move in the direction V. In addition, the fourth link 1024 is pivotally attached to the second link 1022 at point C such that movement of the coupler 405 in the direction Y causes the fourth link 1024 to move in the direction N. FIG. 61B illustrates the final position of the valve repair device 402 after the coupler 405 is moved as shown in FIG. 61A.

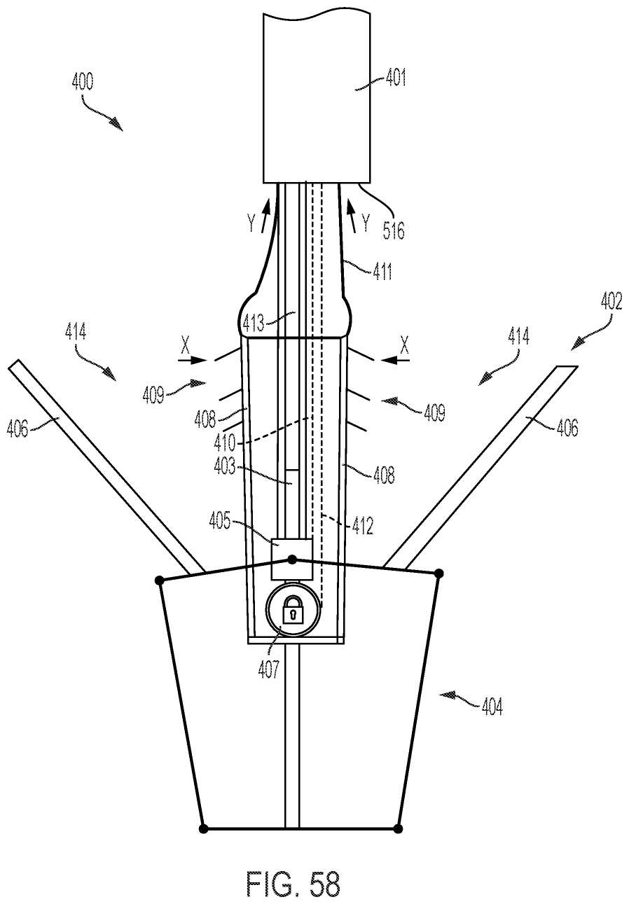

Referring to FIG. 58, the valve repair device 402 is shown in the open position (similar to the position shown in FIG. 61A), and the gripper control mechanism 411 is shown moving the gripping members 408 to provide a wider gap at the opening 414 between the gripping members and the paddles 406. In the illustrated example, the gripper control mechanism 411 includes a line, such as a suture, a wire, etc. that is threaded through an opening in an end of the gripping members 408. Both ends of the line extend through the delivery opening 516 of the delivery device 401. When the line is pulled through the delivery opening 516 in the direction Y, the gripping members 408 move inward in the direction X, which causes the opening 414 between the gripping members and the paddles 406 to become wider.

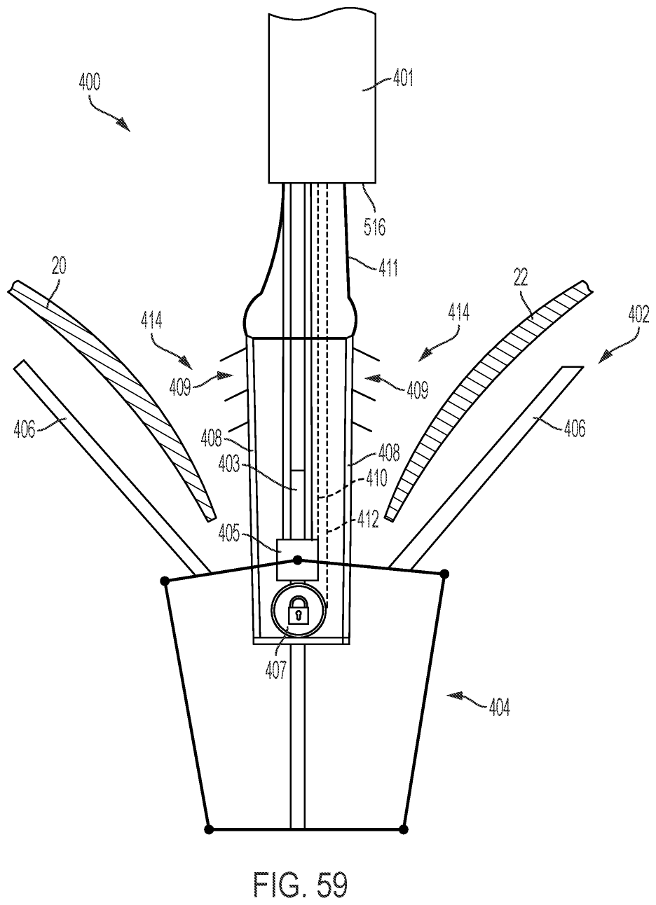

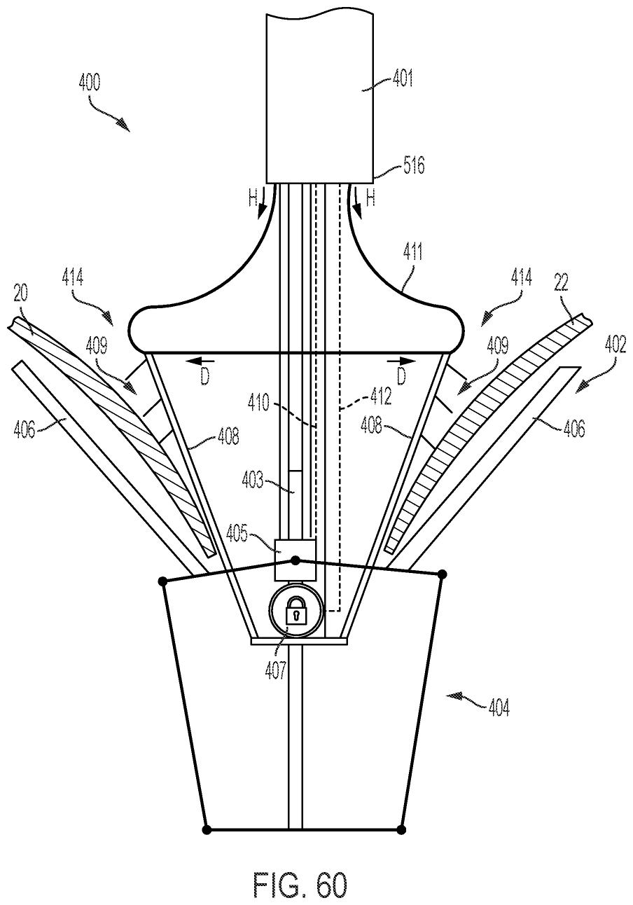

Referring to FIG. 59, the valve repair device 402 is shown such that valve tissue 20, 22 is disposed in the opening 414 between the gripping members 408 and the paddles 406. Referring to FIG. 60, after the valve tissue 20, 22 is disposed between the gripping members 408 and the paddles 406, the gripper control mechanism 411 is used to lessen the width of the opening 414 between the gripping members and the paddles. That is, in the illustrated example, the line of the gripper control mechanism 411 is released from or pushed out of the opening 516 of the delivery member in the direction H, which allows the gripping members 408 to move in the direction D to lessen the width of the opening 414. While the gripper control mechanism 411 is shown moving the gripping members 408 to increase the width of the opening 414 between the gripping members and the paddles 406 (FIG. 59), it should be understood that the gripping members may not need to be moved in order to position valve tissue in the opening 414. In certain circumstances, however, the opening 414 between the paddles 406 and the gripping members 408 may need to be wider in order to receive the valve tissue.

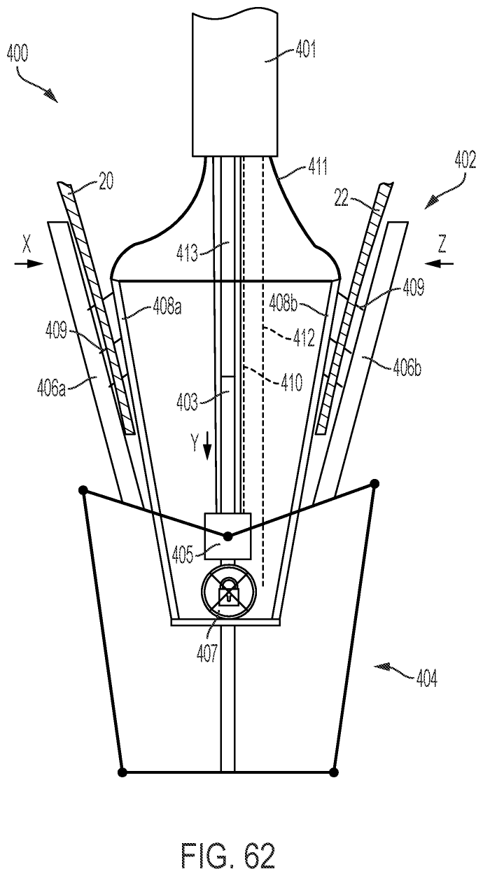

Referring to FIG. 62, the implant or valve repair device 402 is in the closed position and secured to valve tissue 20, 22. The valve repair device 402 is secured to the valve tissue 20 by the paddles 406a, 406b and the gripping members 408a, 408b. In particular, the valve tissue 20,22 is attached to the valve repair device 402 by the barbed portion 409 of the gripping members 408a, 408b, and the paddles 406a, 406b engage the gripping members 408 to secure the valve repair device 402 to the valve tissue 20, 22.

In order to move the valve repair device 402 from the open position to the closed position, the lock 407 is moved to an unlocked condition (as shown in FIG. 62) by the lock control mechanism 412. Once the lock 407 is in the unlocked condition, the coupler 405 can be moved along the shaft 403 by the paddle control mechanism 410. In the illustrated example, the paddle control mechanism 410 moves the coupler 405 in a direction Y along the shaft, which causes one paddle 406a to move in a direction X and the other paddle 406b to move in a direction Z. The movement of the paddles 406a, 406b in the direction X and the direction Z, causes the paddles to engage the gripping members 408a, 408b and secure the valve repair device 402 to the valve tissue 20, 22.

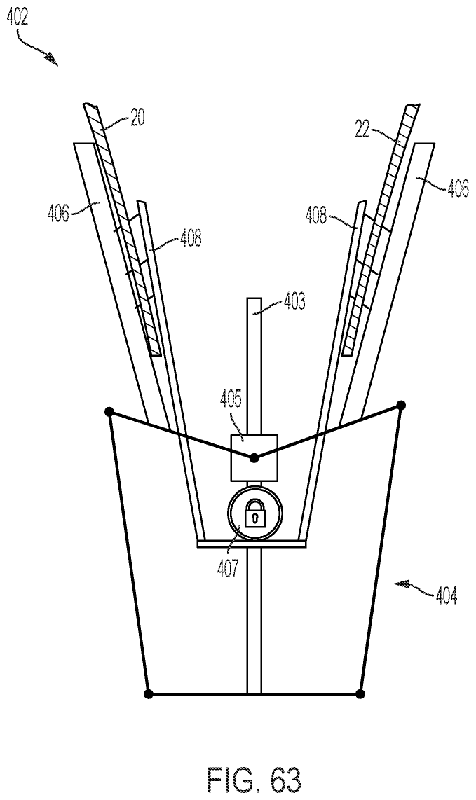

Referring to FIG. 63, after the paddles 406 are moved to the closed position to secure the valve repair device 402 to the valve tissue 20, 22 (as shown in FIG. 62), the lock 407 is moved to the locked condition by the lock control mechanism 412 (FIG. 62) to maintain the valve repair device 402 in the closed position. After the valve repair device 402 is maintained in the locked condition by the lock 407, the valve repair device 402 is removed from the delivery device 401 by disconnecting the shaft 403 from the placement shaft 413 (FIG. 62). In addition, the valve repair device 402 is disengaged from the paddle control mechanism 410 (FIG. 62), the gripper control mechanism 411 (FIG. 62), and the lock control mechanism 412. Removal of the valve repair device 402 from the delivery device 401 allows the valve repair device to remain secured to valve tissue 20, 22 while the delivery device 401 is removed from a patient.

During implantation of an implantable device or implant in the native heart valve, movement of the device to the implanted position may be impeded or obstructed by the native heart structures. For example, articulable portions of an implantable device or implant (such as paddle portions of anchors used to secure the device to the native heart valve tissue) may rub against, become temporarily caught, or be temporarily blocked by the chordae tendineae CT (shown in FIGS. 3 and 4) that extend to the valve leaflets. An example implantable device or implant can be configured to reduce the likelihood of the device or implant getting temporarily caught or blocked by the CT. For example, the implantable device or implant can take a wide variety of different configurations that are configured to be actively or passively narrowed to reduce the width of a paddle frame of an anchor portion of the device and, consequently, reduce the surface area of the device, which will make it easier to move the device/implant past and/or through the CT.

In some implementations, a delivery system or delivery assembly is configured to make it easier to move the implantable device or implant between its various configurations and/or to implant the implantable device or implant in the native heart valve. For example, controls on a handle used in a delivery system/assembly can be configured to enable improved control of the implantable device or implant, as will be described.

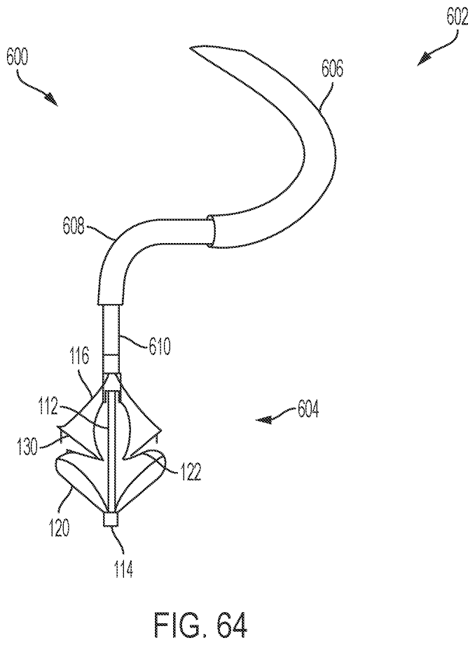

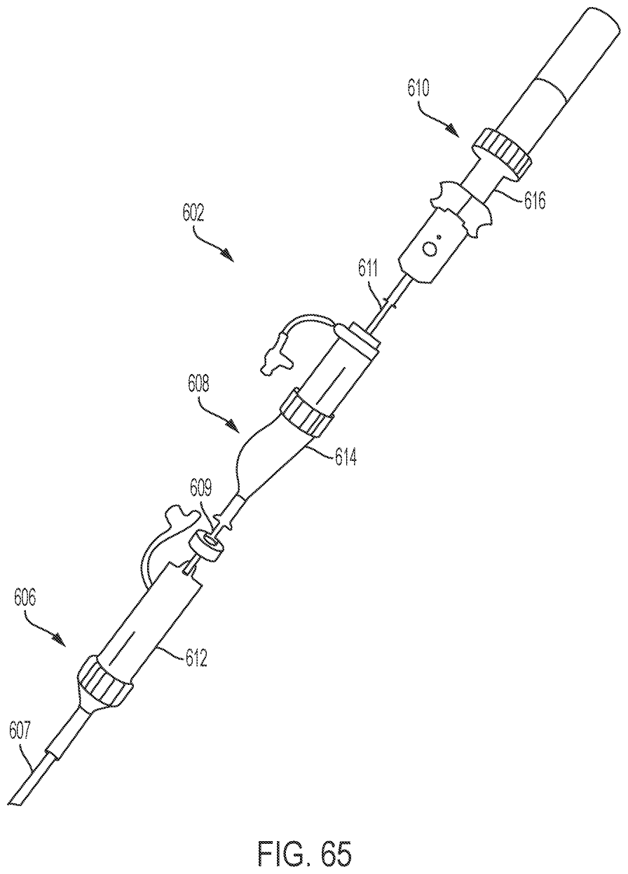

FIGS. 64-65 show an example of a system or assembly 600 (e.g., a valve treatment system or assembly, valve repair system or assembly, valve replacement system or assembly, etc.) and its components. Referring to FIG. 64, the system or assembly 600 can comprise the delivery assembly or delivery system 602 and an implantable device or implant 604. The delivery system 602 can comprise a plurality of catheter assemblies. The delivery system 602 can also comprise one or more optional catheter stabilizers or stabilizing systems/devices (not shown in FIGS. 64 and 65).

In some implementations, as shown in the illustrated example in FIG. 65, the delivery system 602 includes a first catheter assembly 606, a second catheter assembly 608, and a third catheter assembly 610. Though, in some implementation, the delivery system 602 can include fewer or more catheter assemblies than shown. In some implementations, the first catheter assembly 606 is configured as a delivery catheter assembly and will often be referred to as such for illustration herein, though it can also be other types of catheters or catheter assemblies. In some implementations, the second catheter assembly 608 is configured as a steerable catheter assembly and will often be referred to as such for illustration herein, though it can also be other types of catheters or catheter assemblies. In some implementations, the third catheter assembly 610 is configured as an implant catheter assembly and will often be referred to as such for illustration herein, though it can also be other types of catheters or catheter assemblies.

In some implementations, the second catheter assembly or steerable catheter assembly 608 extends coaxially through the first catheter assembly or delivery catheter assembly 606, and the third catheter assembly or implant catheter assembly 610 extends coaxially through the second catheter assembly 608 and the first catheter assembly 606. The implantable device 604 can be releasably coupled to a distal portion of the third catheter assembly or implant catheter assembly 610, as further described below. It should be appreciated that the implantable device 604 can be any device described herein.

As shown in FIG. 65, each of the catheter assemblies (e.g., delivery catheter assembly 606, the steerable catheter assembly 608, and the implant catheter assembly 610) includes a sheath or shaft 607, 609, 611 extending from a handle 612, 614, 616, respectively. The handles 612, 614, 616 are located at a proximal end of each of the corresponding sheaths or shafts, and include one or more control members to enable a user to manipulate the catheter assembly (e.g., bend or rotate the sheath or shaft of the catheter assembly) or control a component coupled to the corresponding catheter assembly (e.g., a control wire extending through the shaft of the catheter assembly).

The delivery catheter assembly 606 and the steerable catheter assembly 608 can be used, for example, to access an implantation location (e.g., a native mitral valve region of a heart) and/or to position the implant catheter assembly 610 at the implantation location. Accordingly, in some implementations, the delivery catheter assembly 606 and the steerable catheter assembly 608 are configured to be steerable. The catheter assemblies or features of the catheter assemblies disclosed by U.S. Pat. Nos. 10,653,862 and 10,646,342 can be used as or in the catheter assemblies 606, 608, 610. U.S. Pat. Nos. 10,653,862 and 10,646,342 are hereby incorporated by reference in their entireties.

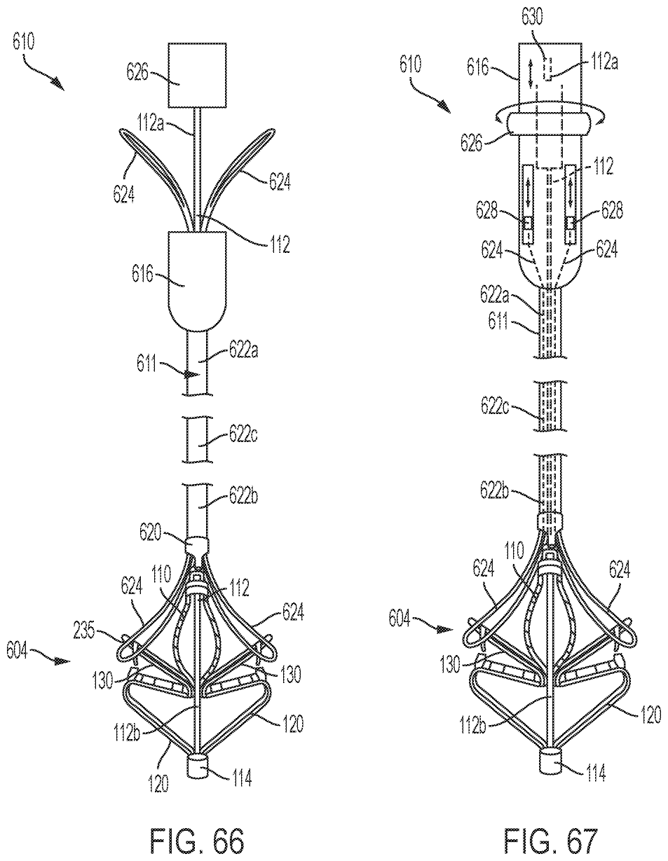

FIGS. 66 and 67 illustrate examples of implant catheter assemblies 610. FIG. 66 illustrates a generalized implant catheter assembly 610 while FIG. 67 is a schematic illustration of an example implant catheter assembly 610, in which each of the clasp actuation lines 624 is coupled to a clasp control member positioned on the handle 616 and the actuation element 112 is coupled (directly or indirectly) to a control element (e.g., knob 626, but the control element can also be a button, switch, slider, motor, button that controls a motor, combination of these, etc.) positioned on the handle. In each of the examples illustrated by FIGS. 66 and 67, the implant catheter assembly 610 can comprise the inner or actuation element 112, a coupler 620, an outer shaft 611, a handle 616 (shown schematically), and clasp actuation lines 624. A proximal end portion 622a of the outer shaft 611 can be coupled to extend distally from the handle 616, and a distal end portion 622b of the outer shaft 611 can be coupled to the coupler 620. The actuation element 112 can extend distally from the control element or knob 626 (shown schematically in FIG. 66), through the handle 616, through the outer shaft 611, and through the coupler 620. The actuation element 112 can be movable (e.g., axially and/or rotationally) relative to the outer shaft 611 and the handle 616. The clasp actuation lines 624 can extend through and be axially movable relative to the handle 616 and the outer shaft 611. The clasp actuation lines 624 can also be axially movable relative to the actuation element 112.

In some implementations, the outer shaft 611 of the implant catheter assembly 610 can be configured to be steerable. For example, although not shown, the implant catheter assembly 610 can comprise an actuation element, such as a pull wire, and a flexible, axially non-compressible pull wire sleeve (e.g., a helical coil).

As shown in FIG. 66, the actuation element 112 of the implant catheter assembly 610 can be releasably coupled to the cap 114 of the device 604. For example, in some implementations, the distal end portion 112b of the actuation element 112 can comprise external threads configured to releasably engage interior threads of the cap 114 of the device 604. As such, rotating the actuation element 112 in a first direction (e.g., clockwise) relative to the cap 114 of the device 604 releasably secures the actuation element 112 to the cap 114, while rotating the actuation element 112 in a second direction (e.g., counter-clockwise) relative to the cap 114 of the device 604 releases the actuation element 112 from the cap 114.

In the examples of FIGS. 66 and 67, the outer shaft 611 of the implant catheter assembly 610 is an elongate shaft extending axially between the proximal end portion 622a, which is coupled to the handle 616, and the distal end portion 622b, which is coupled to the coupler 620. The outer shaft 611 can also include an intermediate portion 622c disposed between the proximal and distal end portions 622a, 622b. The outer shaft 611 can be formed from various materials, including metals and polymers. For example, in some implementations, the proximal end portion 622a can comprise stainless steel and the distal and intermediate portions 622b, 622c can comprise polyether block amide (PEBA). The outer shaft 611 can also comprise an outer covering or coating, such as a polymer that is reflowed over the portions 622a, 622b, and 622c.

As shown in FIGS. 66 and 67, the clasp actuation lines 624 are coupled to the clasps 130 through holes 235 in the clasps 130 and extend axially through the outer shaft 611 between the clasps 130 and the handle 616. In some implementations, e.g., as illustrated by FIG. 67, at the proximal end of the clasp actuation lines 624, the clasp actuation lines 624 are each operatively and/or physically coupled to a clasp control member 628. Each clasp control member 628 is configured such that actuation thereof can cause axial movement of the clasp actuation line 624 relative to the handle 616, outer shaft 611 and/or the actuation element 112. As will be described in greater detail, in some implementations, each of the clasp control members 628 can be actuated/operated independently of the other clasp control member such that each clasp actuation line 624 is moved independently relative to the handle 616, outer shaft 611, the actuation element 112, and/or the other clasp actuation line 624. In some implementations, the clasp control members 628 can be operatively or physically fixed (or synchronized) with respect to one another (e.g., locked) such that the clasp actuation lines 624 are axially moved together relative to the outer shaft 611 and the actuation element 112. In some applications, the clasp control members 628 are configured such that they can be toggled by the end user between independently actuatable and actuatable together (e.g., synchronized).

The clasp control members 628 can be configured in a variety of ways. In some implementations, one or more of the clasp control members 628 is an axially-moving control or slider coupled to a corresponding clasp actuation line 624 to axially move the clasp actuation line 624 relative to the outer shaft 611 and the actuation element 112. In some implementations, one or more of the clasp control members 628 comprises a button, switch, latch, gear, etc.

As described above, in some implementations, the actuation element 112 is coupled at a distal end to the cap 114 of the device 604. The actuation element 112 extends axially through the outer shaft 611 to the handle 616 and is coupled at a proximal end portion 112a to the control element or knob 626. Although described with respect to various figures herein as being configured as a knob, it should be appreciated that the actuation element 112 can be coupled to any other type of control element, such as another type of rotational control member that is rotatable about the axis of the handle 616.

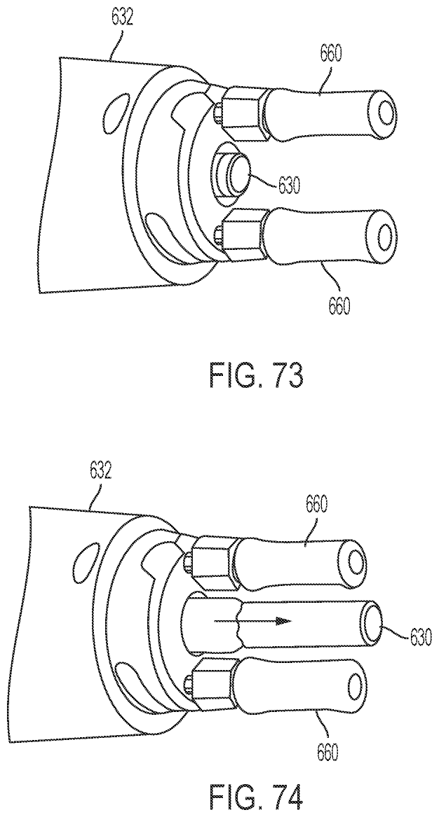

As will be described in greater detail, in some implementations, as the knob 626 is rotated about the axis of the handle 616, the rotation is translated to axial movement of the actuation element 112, and is effective to axially advance or retract the actuation element, such a as a rod or wire, to open or close the valve repair device. Optionally, the knob can also drive a paddle release knob 630 (sometimes referred to as an indicator component) between a proximal, or extended, position (as shown in FIG. 74), and a distal, or retracted, position (as shown in FIG. 73). In some implementations, the control element can be a button, switch, or the like that causes a motor to rotate a shaft, gear, screw, or other component to cause axial movement of the actuation element 112.

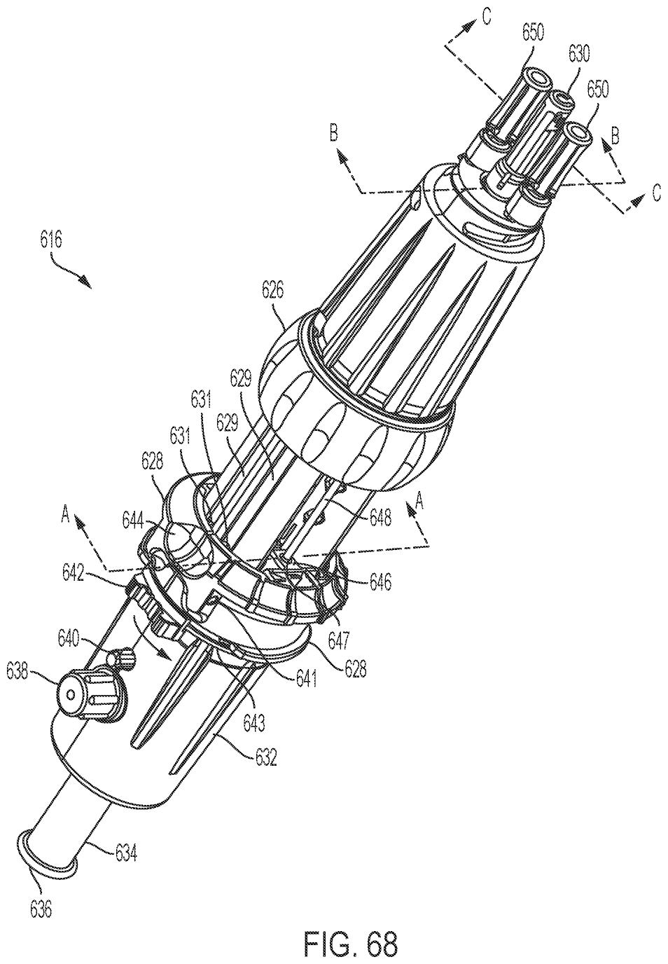

Turning now to FIG. 68, an example of a handle 616 of an implant catheter assembly 610 is shown. In FIG. 68, the handle 616 includes a housing 632 to which the various controls are coupled.

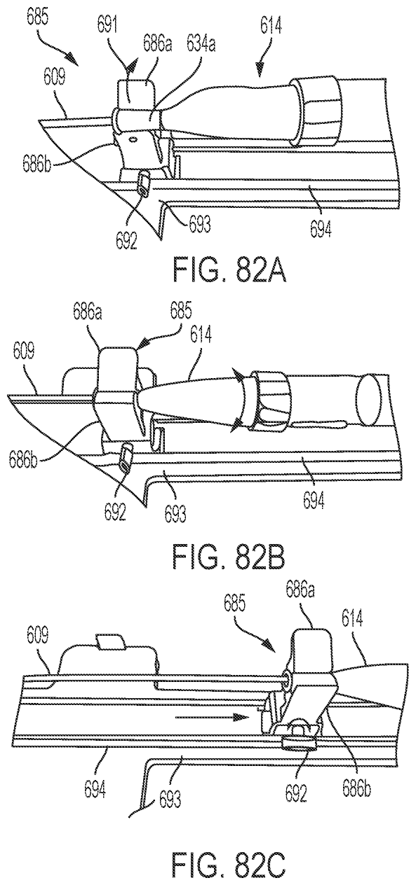

In some implementations, an optional nose grip 634 extends axially from a distal end of the housing 632 and facilitates removably coupling the implant catheter assembly to a stabilizer (shown in FIGS. 82A-C and 83B-D) during use. In some implementations, the nose grip 634 extends between the housing 632 and a distal flange 636, and the distal flange 636 limits the axial movement of the handle with respect to the stabilizer. The housing 632 and the nose grip 634 can be formed from various materials, including polymers such as polycarbonate, and can be formed as a unitary body (e.g., through injection molding) or fastened together in any one of a variety of manners, including fasteners, pins, adhesives, or the like.

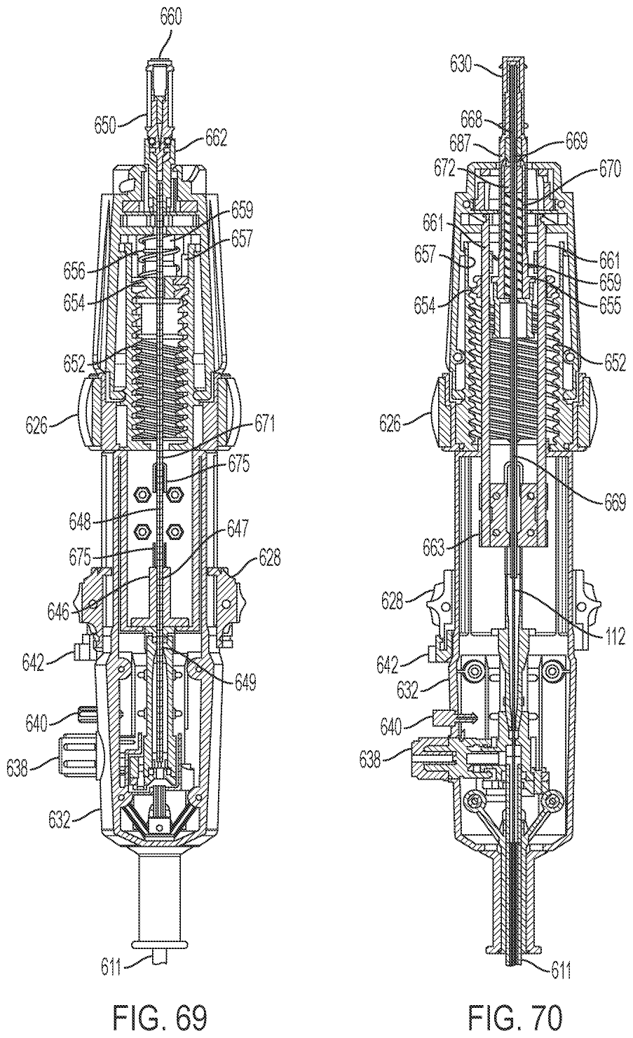

In some implementations, as shown for example in FIGS. 69-71, the housing 632 can comprise open regions or a plurality of lumens, through which the clasp actuation lines 624 and the actuation element 112 extend.

In some implementations, the handle 616 further comprises a flush port 638, as shown in FIGS. 68-70. The flush port 638 is configured for flushing (e.g., with a saline solution) the outer shaft 611 prior to inserting the outer shaft 611 into a patient's vasculature. Additionally, the flush port 638 enables air present in the outer shaft 611 to be removed as fluid is drawn into the proximal end of the outer shaft 611. Additional information regarding a flush port that can be used in the handle 616 can be found in, for example, International Publication No. WO 2020/112622, the entire contents of which is incorporated by reference herein.

In some implementations, as shown for example in FIGS. 68-70, the housing 632 comprises an aperture for receiving a marker/indicator 640 (e.g., a marking pin, tactile marker, visual marker, tag, etc.). The marker/indicator 640 can be a visual and/or tactile indication of which clasp control member is which to help the end user keep track if the handle is rotated.

In some implementations, the marker/indicator 640 can be a marking pin that is removably coupled with the aperture of the housing 632 for storage of the marking pin 640. During use, the marking pin 640 can be removed from the aperture of the housing and inserted into an aperture 641 (shown in FIG. 68) on one of the clasp control members 628. For example, the marking pin 640 can be inserted into an aperture 641 on the clasp control member 628 that controls the clasp to be coupled to the posterior leaflet of the mitral valve. Accordingly, in the event that the handle 616 is rotated during use, the user can readily identify the clasp control member 628 for control of the clasp to be coupled to the posterior leaflet (e.g., the marked clasp control member 628) and the clasp control member 628 for control of the clasp to be coupled to the anterior leaflet (e.g., the unmarked clasp control member 628). Although the marking pin 640 is described as being coupled to the clasp control member 628 that controls the clasp to be coupled to the posterior leaflet, it is contemplated that the marking pin 640 can be coupled to either of the clasp control members 628 to indicate any particular orientation of the implant catheter assembly.

In some implementations, a lock 642 is also included and is configured such that it can be actuated or engaged to selectively physically and/or operatively lock the clasp control members 628 together. The lock can be configured in a variety of ways and take different forms. In some implementations, lock 642 is configured as a slide lock that can be actuated or engaged to slide between a first position, in which the slide lock 642 is coupled to one of the clasp control members 628, to a second position, in which the slide lock 642 is coupled to both of the clasp control members 628. For example, each of the clasp control members 628 can include a flange 643 to which the slide lock 642 is slidably coupled.

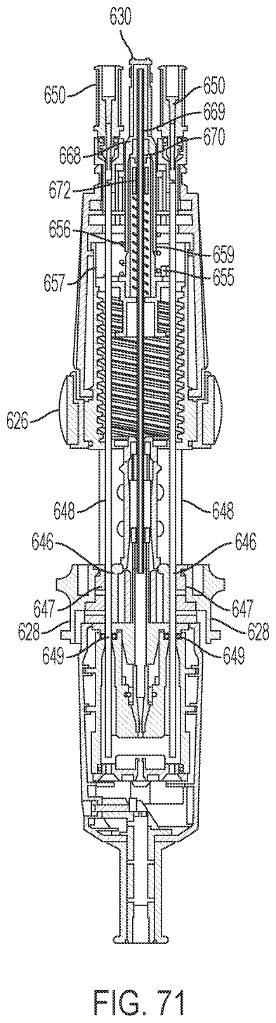

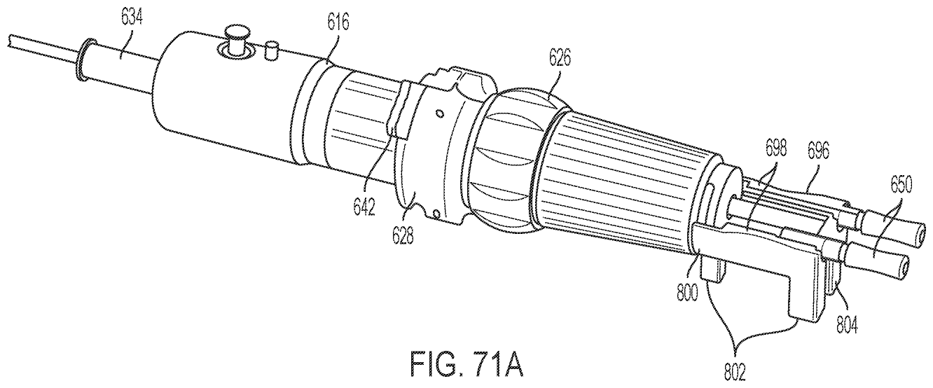

In some implementations, the slide lock 642 can be moved to the first position (shown in FIG. 71A and as indicated by the arrow shown in FIG. 68) in which the slide lock is engaged with the flange 643 of one of the clasp control members 628 (e.g., to enable independent movement of each clasp actuation line relative to the other) and the second position (shown in the FIG. 68) in which the slide lock 642 is engaged with the flange 643 of both of the clasp control members 628 (e.g., to enable simultaneous movement of the clasp actuation lines) in order to selectively control relative movement between the clasp control members 628. When the slide lock is in the first position, each of the clasp control members is axially movable independently of the other clasp control member, and when the slide lock is in the second position, the clasp control members are axially movable together. Although not shown in the figures, in some implementations, each flange 643 can include a stop to limit the position of the slide lock 642 along the flange 643. Moreover, in some implementations, the slide lock 642 can include one or more detents on or near a bottom of the slide lock 642 configured to prevent or inhibit the slide lock 642 from being removed from the flange 643.

In some implementations, each of the clasp control members 628 can include one or more tactile or visual indicators to enable the user to differentiate one clasp control member from the other with improved accuracy. For example, one clasp control member 628 can have a different color and/or texture (e.g., ribbed, smooth) than the other clasp control member 628.

As shown in the figures, in some implementations, each of the clasp control members 628 is wrapped approximately 180 degrees or otherwise in an actuate manner around the circumference of the housing 632 such that together the clasp control members 628 surround and/or encircle (which can include partially encircling) the housing 632. Such an arrangement can, for example, enable the clasp control members 628 to be accessible to the user from any angle with a single hand. Moreover, in the example depicted in FIGS. 68 and 71A, the clasp control members 628 include a depressed region 644 that is generally shaped to receive and cradle the thumb of the user. Although the inclusion of such a depressed region is optional in some examples, it can provide comfort for the user and improve the ease of use of the clasp control members 628 when it is included.

While various configurations and arrangements of clasp control members 628 are possible, having clasp control members that surround, encircle, or are otherwise readily accessible around a significant portion or majority of the handle (or even a full circumference of the handle) provides significant benefits as compared to a handle with clasp control members on only one side or a small portion of the handle. If clasp control members are on only one side of the handle, they may become very difficult to operate and use at various stages of a procedure, for example, when the catheter handle may be rotated to navigate and/or reposition the device appropriately inside the body of a patient and the clasp control members end up on an underside of the handle. Having clasp control members that encircle or are otherwise readily accessible around a significant portion of the handle (or even a full circumference of the handle) makes it significantly easier to continue to operate/actuate the clasp control members when the handle is rotated into any orientation during a procedure.

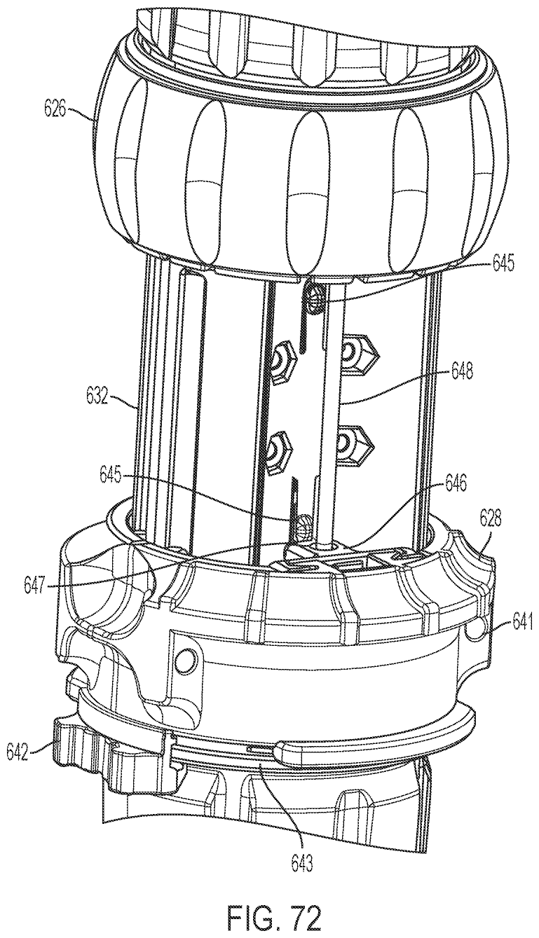

In some implementations, each clasp control member 628 can further comprise one or more keyed projections or tongues that are configured to be received by and slide along a corresponding groove or slot in the housing 632. As best shown in FIG. 72, in some implementations, the housing 632 further includes a pair of detents 645. A first detent 645 is located at a first axial position along a path of one of the clasp control members 628, and a second detent 645 is located at a second axial position along the path of the clasp control member 628. For example, in FIG. 72, one of the detents 645 projects from the housing 632 at a location that places the clasp control member 628 in a fully proximal position (i.e., a fully open position), thereby maintaining the clasp control member 628 in the fully proximal position (i.e., a fully open position) and preventing the clasp control member 628 from moving distally without intention of the user. A second one of the detents 645 projects from the housing 632 at a location that places the clasp control member 628 in a fully distal position (i.e., a fully closed position), thereby maintaining the clasp control member 628 in the fully distal position (i.e., a fully closed position) and preventing the clasp control member 628 from moving proximally without intention of the user. To release the clasp control member 628 for operation of the clasps, the user simply presses the clasp control member 628 with enough force to depresses the detent 645 and slide over the detent 645. When the clasp control member 628 moved to either the proximal position (open) or distal position (closed), the corresponding detent 645 provides tactile and audible feedback to the user indicative that the clasp control member 628 is in the open or closed position.

Referring to FIG. 72, the clasp control member 628 can comprise a coupler 646 configured to attach the clasp control member 628 to a clasp control tube 648. As described in more detail below, the clasp control tube 648 is releasably coupled to the clasp actuation lines 624. Sliding movement of the clasp control member 628 along the housing moves the clasp control tube 648 relative to the housing 632 to open and close the clasps.

In the example illustrated by FIGS. 68 and 69, the coupler 646 includes at least one aperture 647, in which the clasp control tube 648 is connected. The clasp control member 628 is slidably coupled to the housing 632, such as by the mating channels 629 of the housing 632 and the projections 631 of the clasp control member 628 (see FIG. 68) and/or by the clasp control tube 648 extending through a guide passage 649 formed in the housing (see FIG. 71). The clasp control member, the coupler 646, and the clasp control tube 648 can slide together axially (i.e., distally and proximally) with respect to the housing 632 to open and close the clasp. Only a single coupler 646 and clasp control tube 648 can be seen in FIGS. 68 and 69. FIG. 71 shows that each clasp control member 628 is fixedly coupled and slides in conjunction with a corresponding clasp control tube 648 in the same manner. The clasp control tube 648 can be fixed at a proximal end to a suture lock 650 that is used to secure the clasp actuation line 624 to the to the clasp control tube 648.

In some implementations, each clasp control tube 648 can comprise one or more optional keying features 671 at various positions or all along the axial length of the clasp control tube 648. In some implementations, no keying features are included. When included, the optional keying features 671 are complementary to corresponding keying features 675 formed in one or more components of the handle 616, such as in the housing 632 of the handle. These keying features 671, 675 can prevent or inhibit rotation of the clasp control tube 648 in the housing and thereby limit movement of the clasp control tube to linear movement along the length of the housing 632. For example, the optional keying feature 671 of the clasp control tube 648 can comprise a wire welded to the external surface of the clasp control tube 648 (see FIG. 69). In such implementations, one or more portions of the axial path defined by the handle for the clasp control tube 648, can include a corresponding groove configured to receive the wire and provide a path along which the wire can slide. The optional keying features 671, 675 can enhance stabilization of the clasp control member 628 by preventing or inhibiting rotation or torquing of the clasp control tube 648 within the handle 616. For example, the optional keying features 671, 675 can prevent or inhibit rotation or torquing of the clasp control tube 648 within the handle 616 when the control lines are pulled by the clasp control tube 648 to open the clasps and/or the control lines are pulled through the clasp control tube 648 to release the clasps.

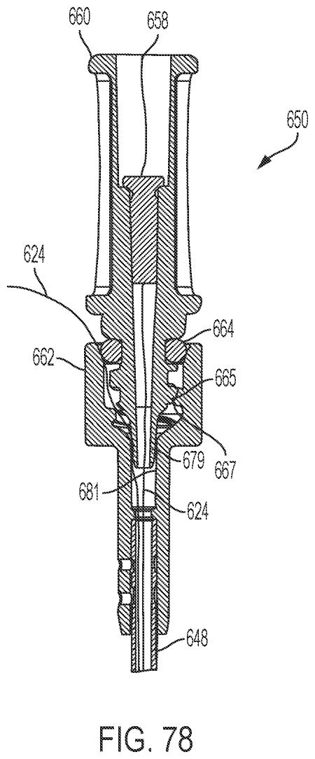

The suture lock can take a wide variety of different forms. In the example illustrated by FIG. 78, the suture lock 650 comprises a post 658, a suture lock body 660, and a suture lock body receptacle 662. In the illustrated example, the suture lock body 660 has external threads 665 that mate with internal threads 667 of the suture lock body receptacle 662 to connect the suture lock body and the suture lock body receptacle together. The suture lock body 660 includes a central bore that extends from a first end to a second end of the suture lock body 660. In some implementations, the central bore of the suture lock body 660 has a diameter that varies from the first end to the second end of the suture lock body 660. At least a portion of the central bore is sized to receive the post 658. Accordingly, the diameter of the central bore of the suture lock body 660 can vary to form a flange to limit the position of the post 658 within the bore of the suture lock body 660 while enabling the post 658 to be inserted into a first end of the suture lock body 660.

In some implementations, the suture lock body receptacle 662 includes a central bore that extends from a first end to a second end of the suture lock body receptacle 662. The central bore of the suture lock body receptacle 662 is sized to receive the suture lock body 660 at threaded end of the suture lock body receptacle 662, and is sized to receive and be attached to the clasp control tube 648 at the second end of the suture lock body receptacle 662. An optional sealing member or o-ring 664 is positioned around the suture lock body 660 to form a fluid-tight seal between the suture lock body 660 and the suture lock body receptacle 662.

In some implementations, the clasp actuation line 624 is fixed at one end of the clasp actuation line 624 to the post 658, which is inserted into the suture lock body 660, thereby coupling the clasp actuation line 624 to the suture lock body 660. In some implementations, the clasp actuation line 624 can be welded, adhered, or otherwise fixedly coupled to the post 658.

In some implementations, the clasp actuation line 624 is threaded through the central bore in the suture lock body 660, through the central bore in the suture lock body receptacle 662, and through the clasp control tube 648. The clasp control tube 648 guides and protects the clasp actuation line 624 through the interior of the handle 616. The clasp actuation line 624 exits the clasp control tube 648 near the distal end of the handle 616 and extends through the outer shaft 611 of the implant catheter assembly 610. As described herein, the clasp actuation line 624 exits the outer shaft 611 at the distal end of the outer shaft, and is coupled to the device, such as by passing through one or more holes 235 in the clasp 130 (see, e.g., FIGS. 66 and 67). The clasp actuation line 624 is then threaded back through the outer shaft 611 from the distal end to the proximal end and through the clasp control tube 648 to form a loop in the clasp actuation line 624 that extends from the distal end of the outer shaft 611. The clasp actuation line 624 then exits the clasp control tube 648 and passes between the suture lock body 660 and the suture lock body receptacle 662 to exit the central bore of the suture lock body receptacle 662. The threaded connection 665, 667 between the suture lock body 660 and the suture lock body receptacle 662 can be tightened to pinch the clasp actuation line 624 in position between a tapered nose 679 of the suture lock body 660 and a reduced diameter passage 681 of the suture lock body receptacle 662. Tightening the threaded connection 665, 667 also compresses the seal, such as an o-ring, between the suture lock body 660 and the suture lock body receptacle 662 to form a fluid-tight seal between the suture lock body 660 and the suture lock body receptacle 662.

In some implementations, an optional spacer or fixture can be used to position the clasp control tubes 648 and/or suture locks 650 while the effective length of the clasp actuation line 624 is set. That is, the optional spacer or fixture sets a correct position of the clasp actuation line 624 corresponding to the clasps in an open position. The clasp actuation lines 624 can then be pulled taught with the clasps in the open position and the suture locks 650 can be tightened to fix the clasp actuation lines 624 to the clasp control tubes 648. The optional spacer or fixture can take a variety of different forms. Any device that sets the position of the control tubes 648 and/or suture locks 650 relative to the body of the handle can be used.

Referring to FIG. 71A, an example clasp setting spacer 696 is shown supporting the clasp control tube 648 and the suture lock 650 for each clasp in a corresponding groove 698 while the threaded connection between the suture lock body and the suture lock receptacle is tightened. Each groove 698 in the clasp setting spacer 696 is sized to receive the clasp control tube 648 and/or at least a part of the suture lock 650. The clasp setting spacer 696 can also include a handle supporting end 800 that is configured to couple to and support the handle 616. In some implementations, the handle supporting end 800 can include a groove that is configured to receive a protrusion on the surface of the handle 616, thereby enabling the clasp setting spacer 696 to be coupled to the handle 616.

As shown in the example in FIG. 71A, the clasp setting spacer 696 can include one or more pairs of optional feet 802. The pair of feet 802 interface with a surface on which the handle is placed during clasp setting and provides support to the handle 616, the clasp control tubes 648, and the suture locks 650.

In some implementations, the clasp setting spacer 696 has a length extending between the handle supporting end 800 and a proximal end 804 of the clasp setting spacer 696. Although the length of the clasp setting spacer 696 can vary, in some implementations, the length of the clasp setting spacer 696 is selected to provide a pre-determined distance between the suture lock and a proximal end of the handle 616. Accordingly, in use, the clasp setting spacer 696 receives the clasp control tube 648 and suture lock 650 and secures them in a predetermined position relative to the handle 616 such that when the clasp control line is secured in place between the suture lock body and the suture lock receptacle, the distance of the path of the clasp control line is constant. This ensures that the clap control line has a sufficient length to enable the clasps to move through their full range of motion while not being too long.

In some implementations, to release the clasp from the clasp actuation line, the suture lock body 660 can be removed (e.g., by unscrewing) from the suture lock body receptacle 662, freeing the end of the clasp actuation line 624 pinched between the suture lock body 660 and the suture lock body receptacle 662. Once the clasp actuation line 624 is released, the clasp actuation line can be pulled through the clasp control tube 648, the outer shaft 611, the hole 235 of the clasp and back through the outer shaft 611 and clasp control tube 648. As such, the clasp actuation line 624 is no longer connected to the clasp and is removed from the patient.

Returning to FIG. 69, in some implementations, as the clasp control member 628 is moved in a proximal direction, the clasp control tube 648 also moves in the proximal direction, pushing the suture lock 650 in the proximal direction with respect to the handle 616. As the suture lock 650 moves in the proximal direction, the loop of the clasp actuation line 624 extending from the distal end of the outer shaft 611 moves proximally, pulling the clasp 130. To release the clasp 130, such as to grasp the leaflet, the clasp control member 628 is moved in a distal direction, which in turn moves the clasp control tube 648 and suture lock 650 in the distal direction, thereby moving the loop of the clasp actuation line 624 in the distal direction, enabling the clasp 130 to move toward the inner paddles 122, grasping the leaflet between the clasp 130 and the inner paddle 122.

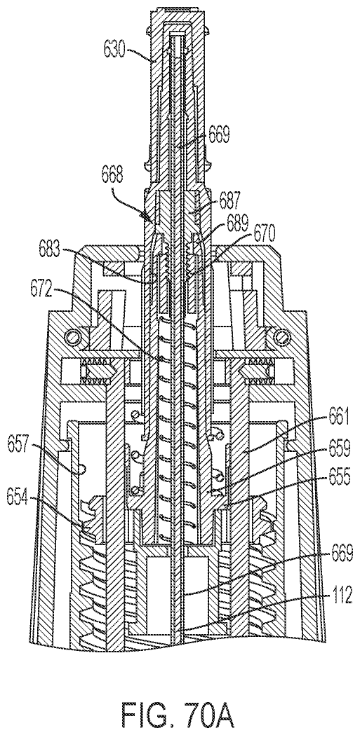

As previously mentioned, in some implementations, the handle 616 can further comprise a control element or knob 626 that can be configured to rotate about an axis of the handle 616 and to control the position of the actuation element 112 relative to the handle 616 and outer shaft 611. As can be seen in the example of FIGS. 69-71, the knob 626 is fixedly coupled to an internally threaded tube 652 positioned within the housing 632 of the handle 616. When the knob 626 is rotated about the axis of the handle 616, the internally threaded tube 652 rotates with respect to the housing 632. An externally threaded nut or retractor 654 is positioned within and engaged with the internally threaded tube 652. The externally threaded nut or retractor 654 and is rotationally fixed with respect to the housing 632. The externally threaded nut or retractor 654 can be rotationally fixed in a wide variety of different ways. In the example illustrated by FIG. 70, a pair of guide rods 661 extend axially within the handle 616, and each of the pair of guide rods 661 is fixed to the housing 632 at both ends of the guide rod 661. A mounting bracket 663 couples the pair of guide rods 661 to the housing 632 at the distal end of each of the pair of guide rods 661. Accordingly, as the internally threaded tube 652 is rotated, the externally threaded retractor 654 is advanced linearly in an axial direction (i.e., distally and proximally) along the pair of guide rods 661.

In some implementations, the externally threaded retractor 654 is connected to the actuation element 112 such that linear movement of the externally threaded retractor 654 causes linear movement of the actuation element 112, thereby moving the device between a fully elongated configuration, an open configuration, and a closed configuration, as described herein. The translation of the rotational movement of the knob 626 into linear motion of the actuation element 112 can result in improved control and precision, thereby leading to improved precision during the opening and closing of the device.

In some implementations, the internally threaded tube 652 includes and unthreaded portion 657. A clutch spring 656 is positioned around an unthreaded proximal portion 659 of the retractor 654. The clutch spring presses against a proximal end surface 655 of the threaded portion of the retractor 654. Proximal movement of the actuation element 112 after closure of the device 604 can result in overclosure or compression of the valve repair device. The position of the unthreaded portion 657 is selected to prevent or inhibit over-retraction of the actuation element 112 and thereby prevent or inhibit over-closing of the valve repair device. That is, the externally threaded retractor 654 is no longer driven proximally when the externally threaded nut or retractor 654 reaches the unthreaded portion 657. The clutch spring is configured to bias the externally threaded retractor 654 distally (e.g., toward the threads of the internally threaded tube 652) when the externally threaded retractor 654 has reached the end of the threads of the internally threaded tube 652. Continued rotation of the knob 626 following disengagement of the external threads of the externally threaded retractor 654 and the internal threads of the internally threaded tube 652 results in an audible indication that the device is in a closed position. The biasing of the externally threaded retractor 654 can also reduce slop in the threaded connection, thereby improving stability of the paddle angle. The biasing of the externally threaded retractor 654 towards the internal threads of the internally threaded tube 652 ensures that the externally threaded retractor 654 will be re-engaged by the internally threaded tube 652 when the knob 626 is rotated in an opposite direction that corresponds to advancement of the actuation element 112.

As shown in FIGS. 70, 70A and 71, in some implementations, the externally threaded retractor 654 can include a central passage 683. A crimp assembly 668 is coupled in the central passage 683. The crimp assembly attaches the actuation element 112 that is in an actuation tube 669 to the paddle release knob 630. The crimp assembly 668 can take a variety of different forms. In the illustrated example, the crimp assembly 668 comprises a collet 687 and a nut 670. The collet 687 includes external threads and the nut includes internal threads. The collet and nut each have a central bore that is sized to receive the actuation tube 669 which surrounds the actuation element 112. The actuation element 112 can be fixed to the actuation tube 669 near the proximal end of the actuation tube 669. The actuation tube 669 is fixed to the crimp assembly 668, which is fixed to the externally threaded retractor 654. The external threads on the outer surface of the collet 687 engage with the internal threads of the nut 670. When tightened, the collet 687 and nut 670 fix the crimp assembly to both the actuation tube 669 and a shoulder 689 at the proximal end of the retractor 654. The nut is positioned within the central passage 683 of the externally threaded retractor 654. A spring 672 is also positioned within the central passage 683 of the externally threaded retractor 654 such that the nut 670 is biased against the shoulder 689. The positioning of the nut 670 against the shoulder 689 allows the collet 687 to be easily attached to the nut 670.

In some implementations, in use, the knob 626 is rotated, which rotates the internally threaded tube 652 to rotate with respect to the housing 632, thereby driving the externally threaded retractor 654 axially. The axial movement of the externally threaded retractor 654 causes axial movement of the actuation element 112, which moves the device between a fully elongated configuration, an open configuration, and a closed configuration. Axial movement of the externally threaded retractor 654 also causes axial movement of the release knob 630 between a proximal, or extended, position (shown in FIG. 74) and a distal, or retracted, position (shown in FIG. 73). Accordingly, in some implementations, the axial position of the release knob 630 with respect to the housing 632 is a visual indicator of the configuration of the device 604.

In some implementations, when the device 604 is in a closed configuration (e.g., shown in FIG. 21), the release knob 630 is in a proximal position, extending from the housing 632, as shown in FIG. 74. To release the device from the implant catheter assembly 610, the release knob 630 is rotated in an unscrewing or loosening direction, which rotates the crimp assembly 668, the actuation tube 669, and the actuation element 112. Rotation of the actuation element 112 extends down the length of the actuation element to the distal end portion 112b of the actuation element, thereby unscrewing the actuation element 112 from the cap 114 of the device 604.

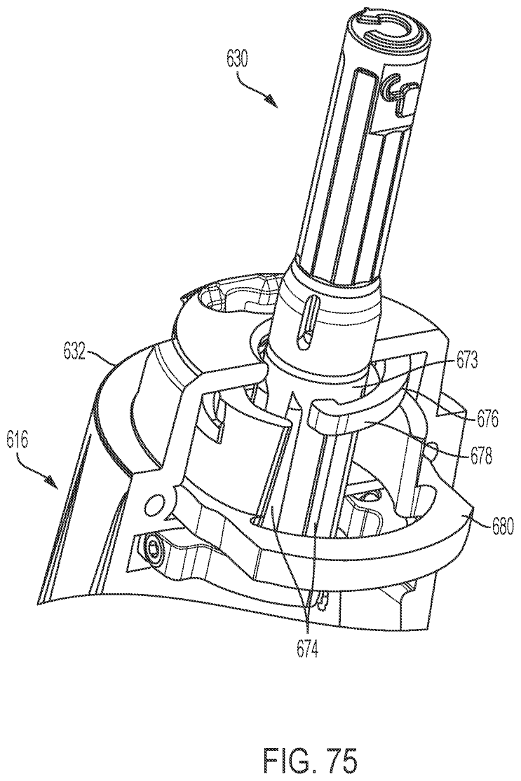

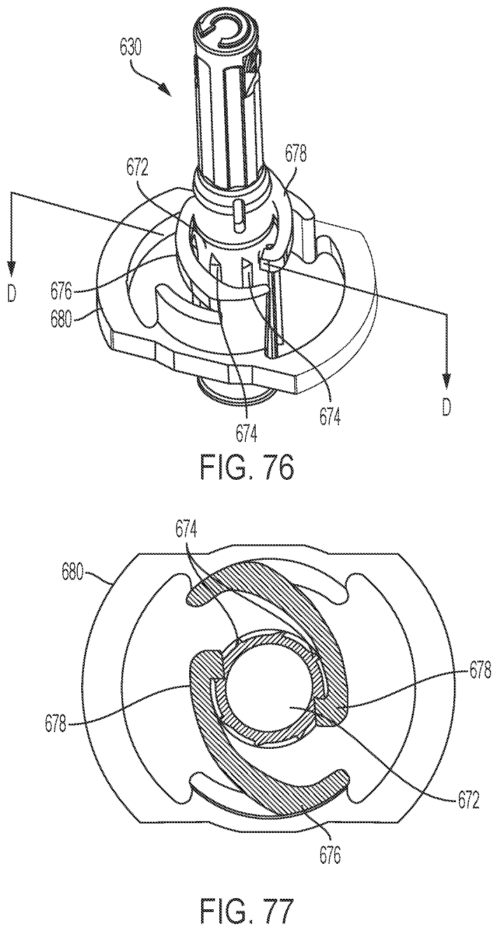

As shown in FIGS. 75-77, in some implementations, the release knob 630 comprises an elongated shaft 673 and a plurality of teeth 674 extending from the outer surface of the elongated shaft 673. Each of the plurality of teeth 674 are uniform, but asymmetrical in shape, with each of the plurality of teeth 674 having a moderate slope on one edge and a much steeper slope on the other edge. A ratchet insert 676 is positioned within the housing 632 of the handle 616, between the elongated shaft 673 and the housing 632. The ratchet insert 676 includes one or more pawls 678 flexibly mounted to a ratchet frame 680. In some implementations, the pawls 678 and the ratchet frame 680 are made from a unitary piece from a material that enables the pawls 678 to flex with respect to the ratchet frame 680.

In some implementations, each of the pawls 678 is in contact with the outer surface of the elongated shaft 673. Accordingly, as the release knob 630 is rotated in the unscrewing or loosening direction, each pawl 678 slides up and over the edge of the tooth 674 with a moderate slope, and springs back into the area between the tooth and an adjacent tooth. In some implementations, the springing back of the pawl into the area between teeth generates an audible indication that a tooth has been cleared. The edge of the tooth 674 with the steeper slope catches the pawl 678 prevents or inhibits the release knob 630 from being rotated in the opposite, tightening direction. Enabling a single direction of rotation of the release knob 630 prevents or inhibits torsion that can build up as a result of the torquing of the actuation element 112 during release of the device 604 from turning the release knob 630 back in the tightening direction.

Once the actuation element 112 is decoupled from the device 604, the actuation element 112 can be withdrawn into the implant catheter assembly 610, and the implant catheter assembly 610 can be withdrawn through the steerable catheter assembly 608 and the delivery catheter assembly 606.

Although not shown in the figures, in some implementations a cap is removably couplable to the proximal end of the handle to cover the release knob 630 and the suture locks 650. Accordingly, the cap can be positioned over the release knob 630 and suture locks 650 to prevent or inhibits the release knob 630 and suture locks 650 from being accidentally contacted or caught on something during manipulation of the implantable device, and removed to access the release knob 630 and suture locks 650.

In some implementations, one or more components of the delivery system are couplable to a stabilizer system to further provide improved control of the delivery system during delivery and implantation of the implantable device. As will be described in greater detail, the stabilizer system generally includes one or more clamps slidable with respect to a base plate that can be fixed relative to the patient. One or more components of the delivery system (e.g., one or more catheter assemblies) are received by the one or more clamps to limit movement of the delivery system or one or more components (e.g., catheter assemblies, etc.) thereof in one or more directions. For example, the stabilizer system can prevent or inhibit the delivery system or component(s) thereof from being moved vertically and from side-to-side while enabling the delivery system or one or more components thereof to be moved axially. In some implementations, the clamps receive a nose grip of one or more of the catheter assemblies of the delivery system.

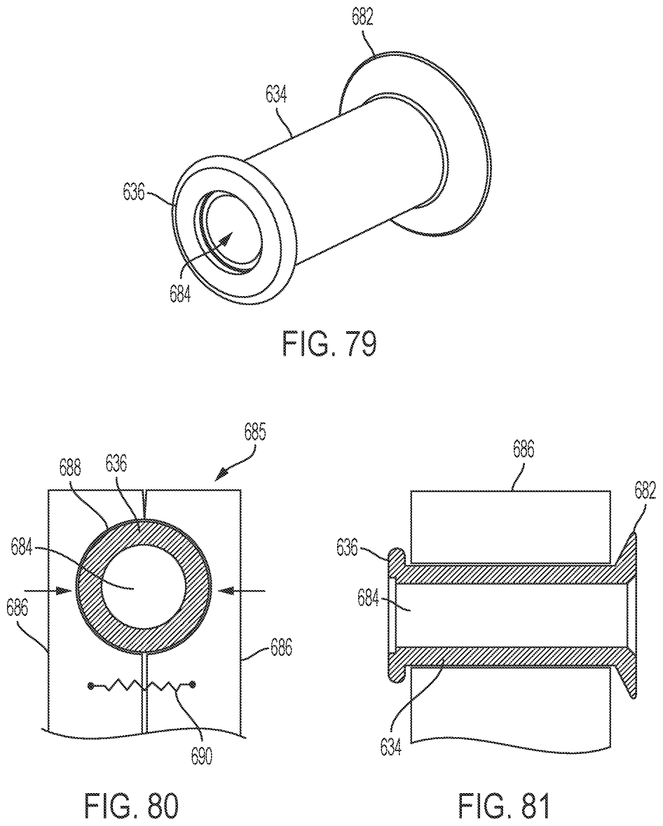

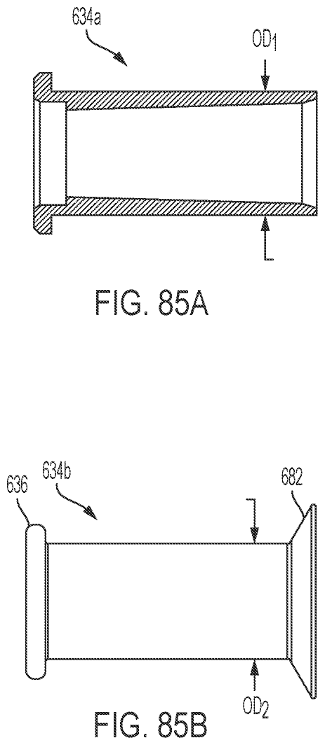

Turning now to FIGS. 79-81, an example nose grip 634 is shown in additional detail. In FIG. 79, the nose grip 634 extends between a distal flange 636 and a proximal flange 682. Although the example shown in FIGS. 79 and 81 includes the proximal flange 682, it is contemplated that some implementations may not include the proximal flange 682, as shown in FIGS. 82A and 85A. Accordingly, the proximal flange 682 is optional. As can be seen in FIGS. 82A-84, the nose grip 634 is coupled at a proximal end of the nose grip 634 to a catheter handle, such as handle 614 of the steerable catheter assembly 608 or handle 616 of the implant catheter assembly 610. A passage 684 extends through the nose grip 634 from the proximal end to the distal end, enabling the corresponding catheter sheath 609, 611 to pass through the nose grip 634.

In some implementations, the nose grip 634 is formed from or as an outer surface that is coated with a rubber or other material with a relatively high coefficient of friction such that the nose grip 634 does not slip when coupled to a clamp 685, such as can be incorporated into a stabilizer. However, in some implementations, the material has a coefficient of friction that allows the nose grip 634 to be rotated about an axis when coupled to the clamp 685, thereby enabling the handle to be rotated during a procedure to rotationally position the implantable device. As shown in FIG. 80, the clamp 685 generally comprises a pair of jaws 686 forming an opening 688 configured to receive the nose grip 634. In some implementations, the pair of jaws 686 are coupled to one another through a spring 690, which biases the pair of jaws 686 toward one another (as shown in FIG. 80), while enabling the jaws 686 to be opened with an application of an opening force to allow the nose grip 634 to be positioned within or removed from the opening 688. U.S. Provisional Patent Application Ser. No. 63/073,392, filed on Sep. 1, 2020 discloses examples of clamps that can be used as the clamp 685 and examples of stabilizing systems and devices that can be used with the systems and devices herein. U.S. Provisional Patent Application Ser. No. 63/073,392 is hereby incorporated by reference for all purposes. Any of the devices, methods, etc. can be used in any of the implementations or examples disclosed by the present application.

As shown in FIGS. 80-81, the nose grip 634 is received in the opening 688 between the jaws 686, with the distal flange 636 and the proximal flange 682 disposed outside the jaws 686. The distal flange 636 has an outer diameter that is greater than an outer diameter of a central portion of the nose grip 634 (e.g., the portion of the nose grip between the distal flange and the proximal flange) and greater than a diameter of the opening of the clamp 685, thereby preventing or inhibiting the nose grip 634 from being pulled out of the clamp 685 unintentionally and from being rotated relative to the clamp 685 unintentionally.

FIGS. 82A-82C depict an example nose grip 634a in conjunction with a handle 614 of a steerable catheter assembly coupled with a clamp 685. In FIGS. 82A-82C, the clamp 685 comprises a movable jaw 686a and a fixed jaw 686b. The clamp 685 is opened and closed by pivoting the movable jaw 686a relative to the fixed jaw 686b. The movable jaw 686a is biased with a spring toward the fixed jaw 686b so that the clamp 685 remains in a closed condition unless opened by an application of an opening force to the movable jaw 686a, as shown by arrow 691 in FIG. 82A. The nose grip 634a is positioned within the opening of the clamp 685 while the clamp is open. Referring to FIG. 62B, when the opening force 691 is removed from the movable jaw 686a, the clamp 685 closes and applies a closing force of the clamp to the nose grip 634a. In some implementations, the closing force applied by the clamp 685 is sufficient to stabilize and prohibit unintended rotation of the nose grip 634a and, accordingly, the handle 614 and corresponding sheath 609. However, intentional rotation of the handle 614, as shown by the rotational arrows in FIG. 82B, can apply a rotational force to the nose grip 634a that is sufficient to overcome the closing force, thereby enabling the handle 614 to be rotated.

In some implementations, the clamp 685 is coupled to a base plate 693 through one or more mounting rails 694 extending from the base plate 693. U.S. Provisional Patent Application Ser. No. 63/073,392 discloses examples of base plates and mounting rails that can be used as the base plate 693 and mounting rails 694, U.S. Provisional Patent Application Ser. No. 63/073,392 and is incorporated by reference herein for all purposes. In some implementations, the one or more mounting rails 694 extend in an axial direction (e.g., proximally and distally) along the base plate 693 and enable the clamp 685 to be moved axially. In some implementations, the clamp 685 is configured to slide with respect to the mounting rails 694 to enable the axial movement of the clamp 685 and, therefore, the steerable catheter assembly when the nose grip 634a is positioned within the opening of the clamp 685. To hold the clamp 685 in a desired location along the mounting rails 694, a locking knob 692 is rotatable between a first, locked position (shown in FIGS. 82A and 82B) and a second, unlocked position (shown in FIG. 82C).

In some implementations, the locking knob 692 is connected to a rotating cam portion (not shown) so that rotating the locking knob 692 causes the rotating cam portion to rotate. As the rotating cam portion is rotated, an oblong portion of the rotating camp portion engages a locking foot (not shown), thereby causing the locking foot to move downward to an extended position, which in turn causes the carriage to move upward with respect to the mounting rails 694. Friction generated between the carriage and the mounting rails 694 retains the clamp 685 in the desired location. To reposition the clamp 685, the locking knob 692 is rotated to disengage the locking foot so that the clamp 685 can slide along the mounting rail 694, as shown in FIG. 82C.

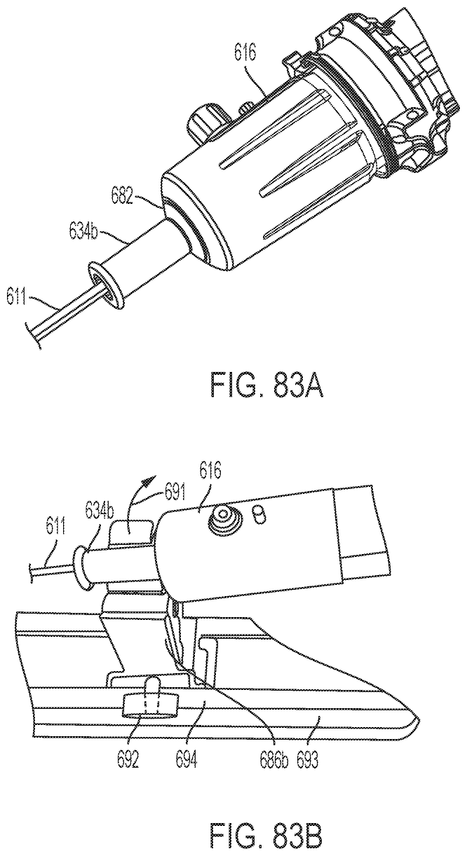

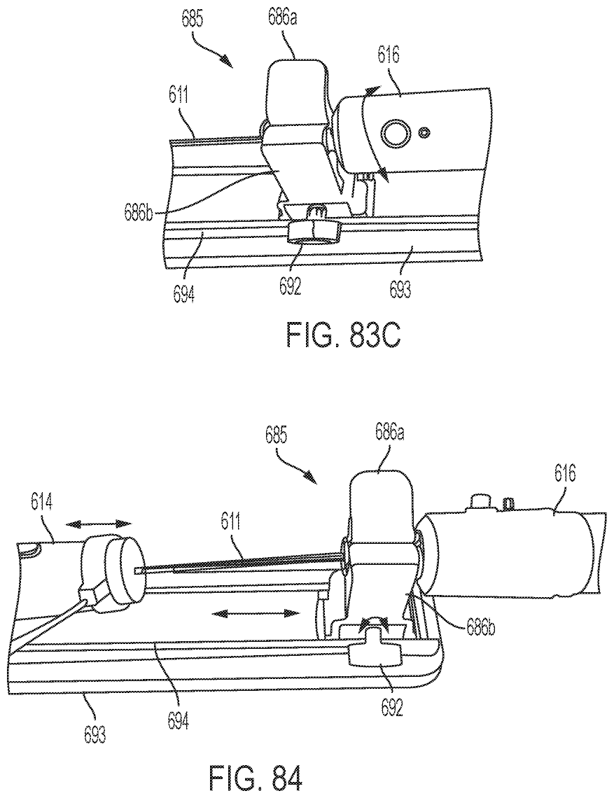

FIGS. 83A-83C depict an example nose grip 634b in conjunction with a handle 616 of implant catheter assembly coupled with a clamp 685. In FIGS. 83A-83C, the clamp 685 comprises a movable jaw 686a and a fixed jaw 686b. The clamp 685 is opened and closed by pivoting the movable jaw 686a relative to the fixed jaw 686b. The movable jaw 686a is biased with a spring toward the fixed jaw 686b so that the clamp 685 remains in a closed condition unless opened by an application of an opening force to the movable jaw 686a, as indicated by arrow 691 in FIG. 83B. The nose grip 634a can positioned within the open clasp. When the opening force is removed from the movable jaw 686a, the clamp 685 closes and applies a clamping force to the nose grip 634a, as shown in FIG. 83C. In some implementations, the closing force applied by the clamp 685 is sufficient to stabilize and prohibit unintended rotation of the nose grip 634a and, accordingly, the handle 616 and corresponding sheath 611. However, intentional rotation of the handle 616, as shown by the rotational arrows in FIG. 83C, can apply a rotational force to the nose grip 634a that is sufficient to overcome the closing force, thereby enabling the handle 616 to be rotated.

As with the previous example, the clamp 685 can be coupled to a base plate 693 through one or more mounting rails 694 extending from the base plate 693. In some implementations, the one or more mounting rails 694 extend in an axial direction (e.g., proximally and distally) along the base plate 693 and enable the clamp 685 to be moved axially. The clamp 685 is configured to slide with respect to the mounting rails 694 to enable the axial movement of the clamp 685 and, therefore, the implant catheter assembly when the nose grip 634a is positioned within the opening of the clamp 685. To hold the clamp 685 in a desired location along the mounting rails 694, a locking knob 692 is rotatable between a first, locked position (not shown) and a second, unlocked position (shown in FIGS. 83B-C).

In some implementations, the locking knob 692 is connected to a rotating cam portion (not shown) so that rotating the locking knob 692 causes the rotating cam portion to rotate. As the rotating cam portion is rotated, an oblong portion of the rotating cam portion engages a locking foot (not shown), thereby causing the locking foot to move downward to an extended position, which in turn causes the carriage to move upward with respect to the mounting rails 694. Friction generated between the carriage and the mounting rails 694 retains the clamp 685 in the desired location. To reposition the clamp 685, the locking knob 692 is rotated to disengage the locking foot so that the clamp 685 can slide along the mounting rail 694.

FIG. 84 depicts a portion of a stabilization system for stabilizing a system or assembly (e.g., a valve treatment system or assembly), such as a system or assembly 600 of FIGS. 64-65. For example, FIG. 84 depicts the handle 616 and sheath 611 of a catheter assembly (e.g., an implant catheter assembly 610), with the sheath 611 thereof being received by a proximal end of the handle 614 of another catheter assembly (e.g., a steerable catheter assembly 608). Although only one catheter assembly (e.g., implant catheter assembly) is shown as being coupled to the stabilization system, it should be appreciated that the handle 614 of the other catheter assembly (e.g., steerable catheter assembly) can also be coupled to a clamp (not shown), e.g., through a nose grip, such as nose grip 634a. Moreover, it should be appreciated that the system or assembly can further include one or more additional catheter assemblies (e.g., a delivery catheter assembly 606) that can also be coupled to a clamp (not shown), e.g., through a nose grip, other attachment point, or the like.

In some implementations, the sheath 611 enters the handle 614 of the steerable catheter assembly and can extend through the handle and into the sheath 609 of the steerable catheter assembly, as described hereinabove. In some implementations, the sheath 611 has a friction fit within the handle 614, the sheath 609, or both. As shown in FIG. 84, the locking knob 692 of the clamp 685 coupled to the nose grip 634 is positioned in an unlocked position, thereby enabling axial movement of the implant catheter assembly with respect to the base plate 693. Accordingly, when the locking knob of the clamp coupled to the nose grip 634 of the handle 614 is also in an unlocked position, axial movement of the handle 616 of the implant catheter assembly can cause axial movement of the handle 614 of the steerable catheter assembly and the handle 616 of the implant catheter assembly with respect to the base plate 693. However, when the locking knob of the clamp coupled to the nose grip 634 of the handle 614 is in a locked position, axial movement of the handle 616 of the implant catheter assembly can cause axial movement of the handle 616 of the implant catheter assembly with respect to the handle 614 of the steerable catheter assembly and the base plate 693.

As described herein, the nose grip 634 can be incorporated into the handles 614, 616 for a steerable catheter assembly and an implant catheter assembly of a delivery system. It is further contemplated that, in some implementations, any one or more of the catheter assemblies included in a delivery system can comprise the nose grip 634 to enable the catheter assembly to be coupled to a stabilization system. Moreover, it is contemplated that in some implementations in which multiple catheter assemblies in a delivery system comprise the nose grip 634, the nose grip 634 can be the same for each catheter assembly or can differ between catheter assemblies. For example, FIGS. 85A and 85B illustrate the nose grip 634a and the nose grip 634b, as described hereinabove. As shown in FIG. 85A, the nose grip 634a, described herein as being incorporated into the handle 614 of the steerable catheter assembly, does not include a proximal flange, whereas the nose grip 634b (FIG. 85B), described herein as being incorporated into the handle 616 of the implant catheter assembly includes both a distal flange 636 and a proximal flange 682.

In some implementations, the nose grip 634a has an outer diameter OD₁, and the nose grip 634b has an outer diameter OD₂. In some implementations, the outer diameter OD₁ is equal to the outer diameter OD₂. In some implementations, the outer diameter OD₁ is different than the outer diameter OD₂. For example, the outer diameter OD₁ can be greater than the outer diameter OD₂, or the outer diameter OD₁ can be less than the outer diameter OD₂. A difference in the outer diameters OD₁, OD₂ can, for example, enable a different tactile feel upon rotation of the steerable catheter assembly as compared to rotation of the implant catheter assembly. In some implementations, the outer diameter OD₁ of the nose grip 634a of the steerable catheter assembly is greater than the outer diameter OD₂ of the nose grip 634b of the implant catheter assembly, which can, for example, provide additional friction between the nose grip 634a and the opening 688 of the clamp 685 as compared to an amount of friction between the nose grip 634b and the opening 688 of the clamp 685. This can provide a feeling to the user that it is easier to turn the implant catheter assembly and implant than it is to rotate the steerable catheter assembly in the guide sheath or delivery catheter assembly.

Although the stabilization system can be effective to stabilize the delivery system at proximal locations, in some implementations, one or more of the catheter assemblies comprise features to further improve accuracy of the delivery of the implantable device. For example, movement of the implant catheter assembly through the steerable catheter assembly or while in the steerable catheter assembly (e.g., rotation of the implant catheter shaft to properly orient the implantable device) can alter the trajectory of the steerable catheter sheath, which in turn alters the location of the implantable device coupled to the implant catheter assembly. Accordingly, in some implementations, features can be implemented to reduce friction between the implant catheter shaft and the steerable catheter shaft, to stiffen a proximal portion of the steerable catheter sheath and/or the implant catheter shaft, or both.

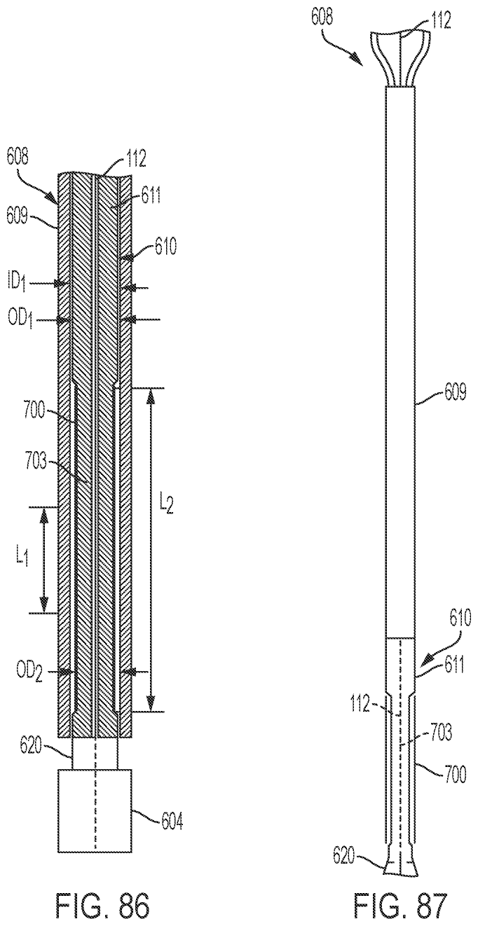

Turning now to FIGS. 86 and 87, a shaft or sheath 611 of a catheter assembly, e.g., an implant catheter assembly 610, having a portion of the shaft or sheath 611 with a reduced outer diameter portion 703 is shown. In particular, FIG. 86 shows a cross-section of a shaft or sheath 611 of a catheter assembly, e.g., an implant catheter assembly 610, extending through a shaft or sheath 609 of another catheter assembly, e.g., a steerable catheter assembly 608. While described in the context of particular types of catheter assemblies for illustration, similar features and principles can be applied in a variety of catheter assemblies.

FIG. 87 shows the shaft or sheath 611 of the implant catheter assembly 610 extending from the shaft or sheath 609 of the steerable catheter assembly 608. As shown in FIG. 86, the shaft or sheath 609 of the steerable catheter assembly 608 has an inner diameter ID₁ through which the shaft or sheath 611 implant catheter assembly 611 extends. The sheath 609 of the steerable catheter assembly 608 includes a steerable portion having a length L₁, over which the sheath 609 of the steerable catheter assembly 608 can be bent or deformed, as described in greater detail above.

In some implementations, the outer shaft or sheath 611 of the implant catheter assembly 610 includes a lumen through which an actuation element 112 (which can be the same as or similar to other actuations elements herein) extends, as described in greater detail above. The actuation element 112 extends in an axial direction through a lumen of the sheath or shaft 611 of the implant catheter assembly 610. In some implementations, the sheath or shaft 611 of the implant catheter assembly has a portion having a first outer diameter OD₁. In some implementations, the portion having the first outer diameter OD₁ extends distally from the handle 616 (FIG. 66) to the reduced diameter portion 703 having a second outer diameter OD₂, but this portion can extend over a smaller distance in some implementations (e.g., may not extend all the way to the handle). The reduced diameter portion 703 is typically proximate the distal end of the sheath 611 of the implant catheter assembly 610. The second outer diameter OD₂ is less than the first outer diameter OD₁ of the sheath of the implant catheter assembly.

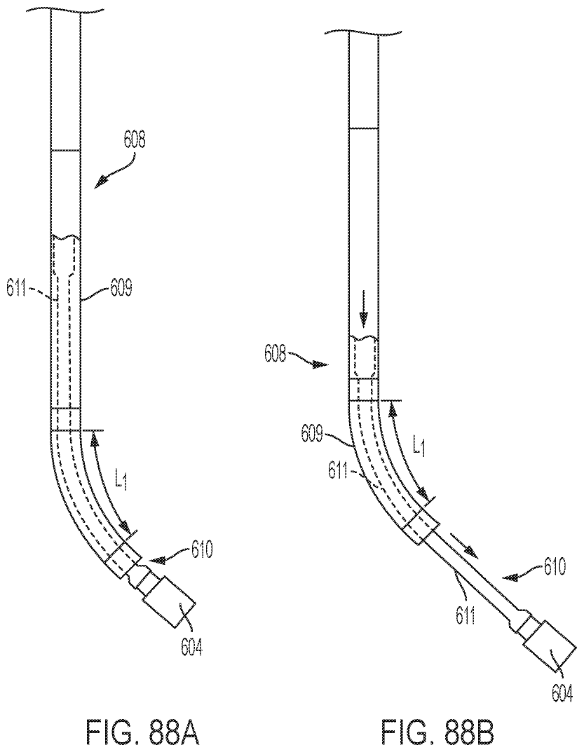

In some implementations, the portion of the sheath 611 of the implant catheter assembly 610 having the first outer diameter OD₁ has a length that is greater than the length L₂ of the reduced diameter portion 703 having the second outer diameter OD₂. The length L₂ of the reduced diameter portion 703 is greater than the length L₁ of the steerable portion of the sheath of the steerable catheter assembly 608. Although the length L₂ of the reduced diameter portion 703 can vary, in some implementations, the length L₂ is greater than or equal to the sum of a stroke distance (i.e., the distance over which the sheath or shaft 611 of the implant catheter assembly 610 is extended from the sheath or shaft 609 of the steerable catheter assembly 608 during delivery of the implantable device) and the length L₁ of the steerable portion of the sheath or shaft 609 of the steerable catheter assembly 608. Accordingly, the system can be operated such that only the reduced diameter portion 703 of the sheath or shaft 611 is in the steerable portion of the sheath or shaft 609 during use of the implant catheter assembly 610 and the steerable catheter assembly 608 to position and implant the implant or valve repair device 604. The sheath or shaft 611 of the implant catheter assembly 610 can have a reduced outer diameter over the entire length of the sheath or shaft 611 that will be within the steerable portion of the sheath 609 of the steerable catheter assembly 608, including when the sheath or shaft 611 implant catheter assembly is retracted and extended with respect to the steerable catheter assembly 608, as shown in FIGS. 88A and 88B.

The difference between the first outer diameter OD₁ and the second outer diameter OD₂ can vary depending on the particular implementation and can be, for example, from about 0.25 mm to about 0.76 mm, or any range between 0.25 mm and 0.76 mm. In some implementations, the transition from the first outer diameter OD₁ to the second outer diameter OD₂ is gradual, and can form a smooth taper from the first outer diameter OD₁ to the second outer diameter OD₂ over a distance of from about 25 mm to about 50 mm. In some implementations, one or more discrete steps are defined from the first outer diameter OD₁ to the second outer diameter OD₂. Moreover, as shown in FIGS. 86 and 87, the sheath or shaft 611 of the implant catheter assembly 610 transitions from the first outer diameter OD₁ to the second outer diameter OD₂ and back to the first outer diameter OD₁ along the length of the sheath or shaft 611 of the implant catheter assembly 610, such that the portion having the second outer diameter OD₁ is between two portions of the implant catheter assembly having the first outer diameter OD₁.

In some implementations, both of the outer diameters OD₁ and OD₂ are less than the inner diameter ID₁ of the sheath 609 of the steerable catheter assembly 608, and, as described above, the sheath 611 of the implant catheter assembly 610 has the second outer diameter OD₂ along the length L₁ of the steerable portion of the sheath 609 of the steerable catheter assembly 608. The reduced outer diameter OD₂ of the sheath 611 of the implant catheter assembly 610 through the steerable portion of the sheath 609 of the steerable catheter assembly 608 reduces friction between the steerable catheter sheath 609 and the implant catheter sheath 611, which can reduce the amount of cross-talk between the steerable catheter sheath 609 and the implant catheter sheath 611 (e.g. unintended flexing of the steerable catheter sheath 609 and the implant catheter sheath 611 due to friction therebetween), and reduce the need to re-orient the steerable catheter assembly during implant positioning.

As an alternative to reducing the outer diameter of the sheath of the implant catheter assembly over the steerable length, it is contemplated that, in some implementations, the inner diameter of the sheath of the steerable catheter assembly can be increased over the length L₁ while the outer diameter of the sheath of the implant catheter assembly remains constant. The inner diameter of the portion of the sheath 609 that flexes can be increased by an amount of from about 0.25 mm to about 0.76 mm (or any subrange thereof), and/or the transition from the first inner diameter to the second inner diameter can be over a distance of about 25 mm to about 50 mm. Moreover, in some implementations, both the inner diameter of the sheath of the steerable catheter assembly can be increased and the outer diameter of the sheath of the implant catheter assembly can be decreased to reduce friction between the steerable catheter assembly and the implant catheter assembly through the steerable portion.

Friction can additionally or alternatively be reduced between the implant catheter shaft and the steerable catheter sheath through the use of a lubricated coating 700, as shown in FIGS. 86 and 87. The lubricated coating can take a wide variety of different forms. The coating can be a wet or dry coating or include both wet and dry components. In FIGS. 86 and 87, the lubricated coating 700 is applied to an outer surface of the reduced diameter portion 703 of the sheath 611 of the implant catheter assembly 610 having the second outer diameter OD₂. However, it is contemplated that some implementations can include the lubricated coating 700 over a length of a sheath of the implant catheter assembly having a substantially constant outer diameter. Alternatively or additionally, the lubricated coating 700 can be applied to an inner surface of the length L₁ of the steerable portion of the sheath 609 of the steerable catheter assembly 608. Moreover, although illustrated in FIGS. 86 and 87 as being included along only the length L₂, it is contemplated that the coating can be applied along any additional length of the sheath 611 of the implant catheter assembly 610 or the sheath 609 of the steerable catheter assembly 608.

The lubricated coating 700 can be a coating made from a wide variety of different materials. Any material that reduces friction can be used. The lubricated coating 700 can be a coating made, for example, from silicone oil, a hydrophilic material, or another material having a low coefficient of friction, such as perfluoropolyether (PFPE) or expanded polytetrafluoroethylene (ePTFE). Accordingly, the lubricated coating 700 can include hydrophilic coatings, coatings of PFPE lubricants, and ePTFE sleeves, such as coatings made from the materials commercially available under the tradenames CHRISTO-LUBE™ (available from Engineered Custom Lubricants) and SURMODICS SERENE™ (available from Surmodics, Inc.).

In some implementations, the lubricated coating 700 reduces a coefficient of friction between the outer surface of the sheath 611 of the implant catheter 610 and an inner surface of the sheath 609 of the steerable catheter assembly 608 through the steerable portion of the sheath 609 of the steerable catheter assembly 608. This reduction in friction can reduce the amount of cross-talk between the steerable catheter sheath 609 and the implant catheter sheath 611 (e.g., unintended flexing of the steerable catheter sheath 609 and the implant catheter sheath 611 due to friction therebetween), and reduce the need to re-orient the steerable catheter sheath during implant orientation.

Use of a liner (e.g., a lubricious liner) within the lumen of the steerable catheter sheath 609 can alternatively or additionally reduce friction between the steerable catheter sheath 609 and the implant catheter sheath 611. In some implementations, such a liner is formed from a polyamide doped with a PTFE powder. The PTFE powder can be incorporated into molten polyamide in an amount of from about 5 wt. % to about 30 wt. %, depending on the particular implementation. For example, the PTFE powder can be incorporated into the polyamide in an amount of from about 5 wt. % to about 30 wt. %, from about 5 wt. % to about 25 wt. %, from about 5 wt. % to about 20 wt. %, from about 5 wt. % to about 15 wt. %, from about 7.5 wt. % to about 30 wt. %, from about 7.5 wt. % to about 25 wt. %, from about 7.5 wt. % to about 20 wt. %, from about 7.5 wt. % to about 15 wt. %, or from about 10 wt. % to about 30 wt. %, from about 10 wt. % to about 25 wt. %, from about 10 wt. % to about 20 wt. %, or from about 10 wt. % to about 15 wt. %, including any ranges and sub-ranges therein.

The polyamide can be a variety of different polyamides. In some implementations, the polyamide is nylon 6,6, nylon 6,12, nylon 4,6, nylon 6, nylon 12, or a combination thereof. In some implementations, the polyamide has a Shore D durometer of 70D or greater. For example, the polyamide can have a Shore D durometer of 70D or greater, 75D or greater, 80D or greater, or 85D or greater. According to various implementations, the PTFE-doped polyamide liner may exhibit a coefficient of friction that is less than that of an otherwise identical polyamide liner, while also exhibiting an improved adhesion to a polymer jacket (e.g., a PEBAX or nylon polymer jacket) bound to the outer surface of the polymer liner. The PTFE-doped polyamide liner can define an interior surface of one or more lumens of the guide sheath, the steerable catheter, and/or the implant catheter of the delivery assemblies described herein, or of any other delivery assembly.

According to some implementations, the inner diameter ID₁ of the sheath 609 of the steerable catheter assembly 608 varies along the length of the sheath 609 and, more particularly, can have a larger inner diameter at or near the ends of the sheath 609 than at an inner diameter at a point located closer to the center of the length of the sheath 609. For example, in some implementations, the inner diameter ID₁ of the sheath 609 of the steerable catheter assembly 608 is flared at one or both of the proximal end and the distal end of the sheath. The flared inner diameter ID₁ of the sheath 609 can be effective to create a smooth transition between the outer diameter of the sheath 611 of the implant catheter assembly 610 and the outer diameter of the sheath 609 of the steerable catheter assembly 608, which in turn can reduce an amount of coating (e.g., the lubricated coating 700) scraped off of the outer surface of the sheath 611 of the implant catheter by the inner surface of the sheath 609 of the steerable catheter assembly 608.

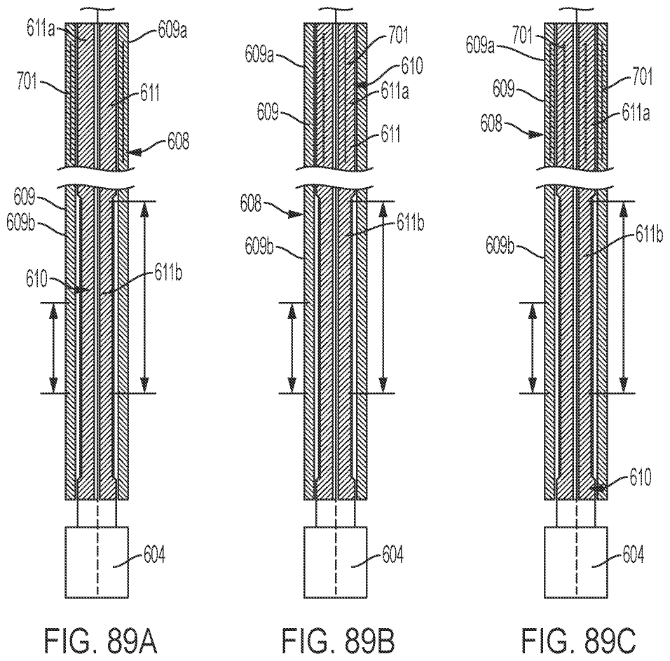

As described above, another method of improve accuracy of the delivery of the implantable device includes stiffening a proximal portion of the sheath 609 of the steerable catheter assembly 608 (as shown in FIG. 89A), the sheath 611 of the implant catheter assembly 610 (as shown in FIG. 89B), or both (as shown in FIG. 89C). It is believed that stiffening the proximal portion of the sheaths of the catheter assemblies can reduce torsional deformation, thereby reducing lag between the implant catheter handle rotation and rotation of the implant, reducing the out of plane movement of the sheath of the steerable catheter assembly, or both, depending on the sheath of the catheter assembly that is stiffened.

In FIGS. 89A-C, a proximal portion 611a of the sheath or shaft 611 of the implant catheter assembly 610 and a distal portion 611b of the sheath or shaft 611 of the implant catheter assembly 610 are shown extending through a proximal portion 609a of the sheath 609 of the steerable catheter assembly 608 and a distal portion 609b of the sheath 609 of the steerable catheter assembly 608, respectively. The proximal portion 611a of the sheath or shaft 611 of the implant catheter assembly 611, the proximal portion 609a of the sheath 609 of the steerable catheter assembly 608, or both comprise a stiffening material 701, while the distal portions 609b, 611b of the sheaths 609, 611 of the implant catheter assembly 610 and steerable catheter assembly 608 do not include the stiffening material. Accordingly, the proximal portion of the sheath(s) or shafts(s) of the catheter assembly(s) including the stiffening material 701 is/are stiffened as compared to the distal portion(s) of the sheath(s) or shaft(s) of the catheter assembly(s).

The stiffening material 701 can take a wide variety of different forms. The stiffening material 701 can be, for example, a laser-cut hypotube, a material having a higher durometer than materials in the distal portion(s) of the sheath(s) or shaft(s) of the catheter assembly(s), one or more braids, one or more meshes, one or more woven materials, or the like. The stiffening material 701 can be incorporated into the catheter sheath(s) or shaft(s) as a layer of a multi-layered sheath, as shown in FIGS. 89A-C. In the example illustrated by FIG. 89A, the stiffening material 701 is provided in the proximal portion 609a of the steerable catheter shaft or sheath 609. In the example illustrated by FIG. 89B, the stiffening material 701 is provided in the proximal portion 611a of the implant catheter shaft or sheath 611. In the example illustrated by FIG. 89C, the stiffening material 701 is provided in the proximal portion 609a of the steerable catheter shaft or sheath 609 and in the proximal portion 611a of the implant catheter shaft or sheath 611.

FIG. 98 is another illustration of an example implant catheter. As shown in FIG. 98, the implant catheter 611 includes stiffening material in the form of a braid, mesh, or woven material 704 and a laser-cut hypotube 702 extending over the braid, mesh, or woven material 704. As described above, the laser-cut hypotube 702 can have a variable or a constant cut pattern along a length of the implant catheter 611. The braid, mesh, or woven material 704 can extend over a polymer layer 732 (e.g., a PEBA layer) that defines one or more lumens of the implant catheter 611. Although illustrated in FIG. 98 as including five lumens, it is contemplated that the polymer layer 732 can include one or more lumens depending on the particular implementation. The implant catheter illustrated in FIG. 98 also includes a polymer jacket 734, which can impart lubricity, stiffness, or other properties to the implant catheter 611. One or more additional layers can also be included in some implementations, including but not limited to PTFE liners, polymer layers, adhesive layers, or the like.

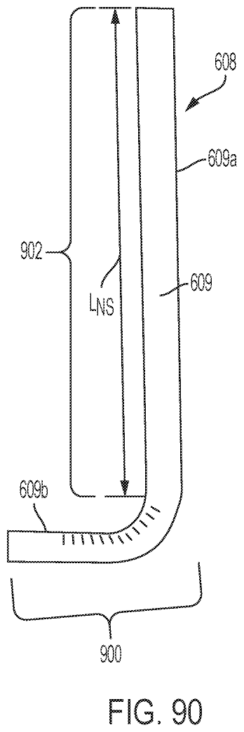

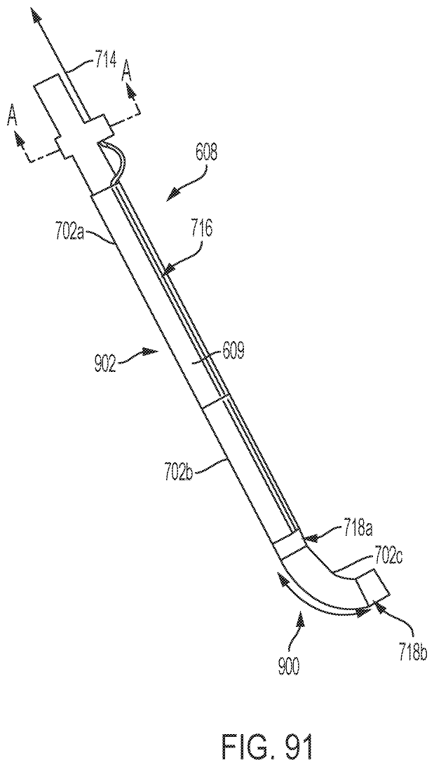

FIGS. 90 and 91 illustrate an example of a steerable catheter assembly 608. The steerable catheter assembly 608 includes a steerable portion 900 and a portion that is not steerable 902. In some implementations, the stiffness of the sheath of the catheter assembly can vary along the length L_{N}S of the sheath. Generally, the non-steerable portion is configured to have a greater stiffness at a proximal end than at a distal end. The stiffness of the non-steerable portion 902 can be varied in a variety of different ways. The variable stiffness non-steerable portion 902 can be used with any of the implementations disclosed by the present application or can be used in a delivery system that does not include any of the other features disclosed by the present application. The variable stiffness non-steerable portion can be used in conjunction with a decreased outer diameter of the distal portion of the sheath or shaft 611 of the implant catheter assembly 610 and/or a low-friction coating can be on the decreased outer diameter section of the sheath or shaft 611 of the implant catheter assembly 610. Further, the sheath 607 of the guide sheath or catheter assembly 606 can include a variable stiffness non-steerable portion in addition to or instead of the variable stiffness non-steerable portion of the sheath or shaft of the steerable catheter assembly 608.

As shown in FIG. 90, in some implementations, when a non-steerable portion 902 of the sheath or shaft 609 of the steerable catheter assembly 608 has a variable stiffness, such as by incorporation of a laser-cut hypotube, the variable stiffness portion can extend any length of the non-steerable portion 902. Accordingly, in some implementations, a variable stiffness length of the sheath 609 of the steerable catheter assembly 608 is less than or equal to a length L_{NS}. In some implementations, this stiffened length can include one or more stiffnesses.

A variable stiffness can be achieved in a variety of different ways. In the example illustrated by FIG. 91, a variable stiffness can be achieved, for example, by using a first laser-cut hypotube 702a along a first length of the sheath 609 of the catheter assembly 608 and a second laser-cut hypotube 702b along a second length of the sheath 609 of the catheter assembly, where the first laser-cut hypotube 702a and the second laser-cut hypotube 702b have different stiffnesses. An example catheter that includes one or more laser-cut hypotubes is disclosed by PCT Application No. PCT/US2019/062194, published as WO2020106705A1, filed on Nov. 19, 2019, which is incorporated herein by reference. Any of the features of the devices and systems disclosed by PCT Application No. PCT/US2019/062194 can be used with any of the implementations of the present application.

In FIG. 91, an actuation element, such as a pull wire 714 extends along the sheath 609 (typically embedded in the material of the sheath) of the steerable catheter assembly 608 and extends through a compression coil 716 (also typically embedded in the material of the sheath). The compression coil 716 (along with the pull wire 714) extend along (either inside or outside) the first laser-cut hypotube 702a and the second laser-cut hypotube 702b. In some implementations, each of the first laser-cut hypotube 702a and the second laser-cut hypotube 702b are formed as jackets that cover the compression coil 716. The compression coil 716 is affixed (e.g., welded or adhered) to a first ring 718a. At the first ring 718a, the pull wire 714 exits the distal end of the compression coil 716 and extends between the first ring 718a and a second ring 718b, and is affixed (e.g., welded or adhered) to the second ring 718b. In some implementations, the first ring 718a and the second ring 718b are at opposite ends of a third laser-cut hypotube 702c having a flex pattern cut into it to enable the sheath 609 of the steerable catheter assembly 608 to flex along the length of the third laser-cut hypotube 702c. Accordingly, to steer the sheath of the steerable catheter assembly 608, the pull wire 714 is pulled in a proximal direction, which causes the second ring 718b to move relative to the first ring 718a.

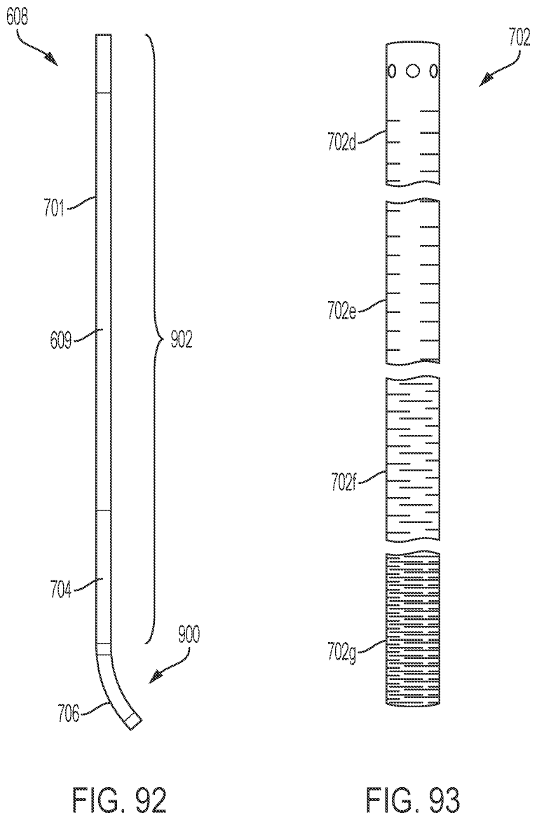

The sheath 609 of the steerable catheter assembly 608 illustrated in FIG. 92 or other catheter assemblies can also have a variable stiffness. A proximal portion 701 of the sheath of the catheter assembly can comprise a laser-cut hypotube positioned over a braid (or other mesh or woven material) which surrounds the tubing or polymer material of the sheath 609 of the catheter assembly 608. The sheath 609 of the steerable catheter assembly 608 also includes a portion 704 in which no laser-cut hypotube is present, but the braid surrounds the tubing and a portion 706 in which the tubing is not covered by the laser-cut hypotube or the braid. Accordingly, the sheath 609 of the steerable catheter assembly 608 in FIG. 92 has at least three stiffnesses along its length, where each of the hypotube and the braid add to the stiffness of the tubing 706.

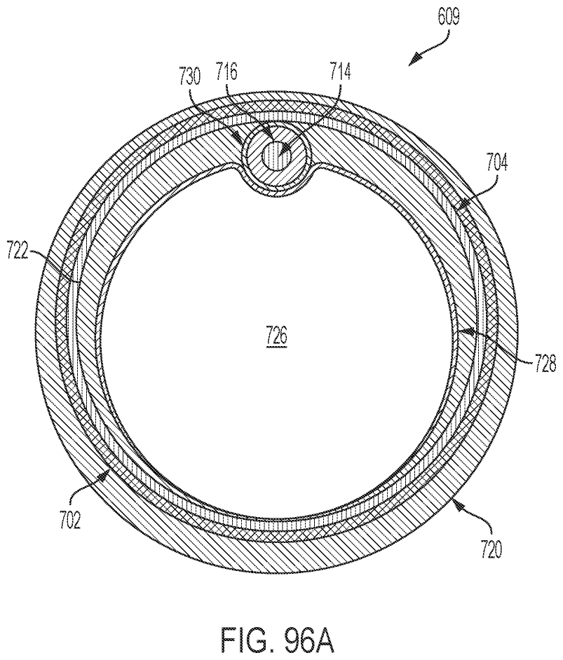

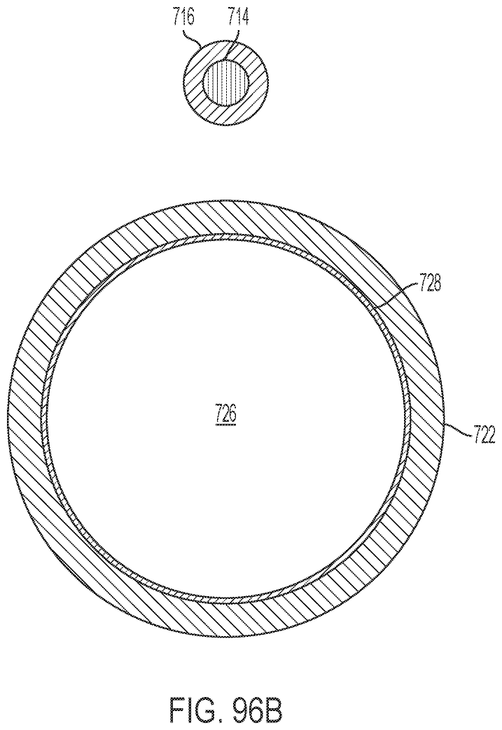

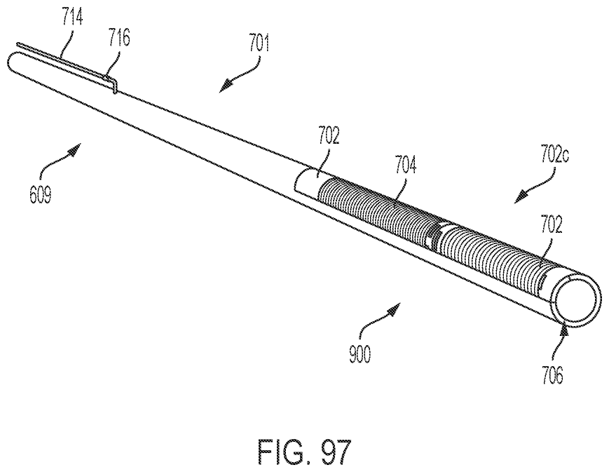

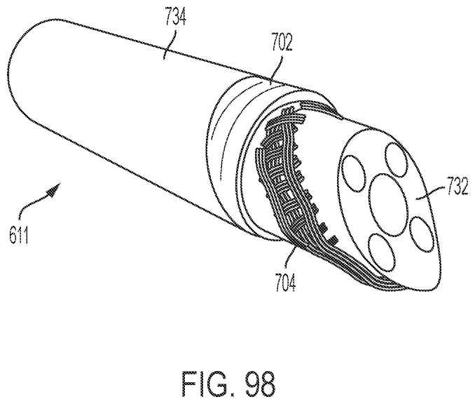

Referring to FIG. 96A and FIG. 97, in some implementations, the sheath 609 includes an elongated middle portion that comprises a braid, mesh, or woven material 704 positioned within a laser-cut hypotube 702. In some implementations, the braid, mesh, or woven material 704 and the laser-cut hypotube 702 are embedded in fused tubing or polymer material 706 of the sheath 609. In the example illustrated by FIG. 97, the sheath 609 also includes a steerable portion 900 that comprises another laser-cut hypotube 702c. The braid, mesh, or woven material 704 and the laser-cut hypotube 702 are not included in the steerable portion 900. The laser-cut hypotube 702c has a flex pattern cut into it to enable the sheath 609 to flex along the length of the third laser-cut hypotube 702c. Accordingly, to steer the sheath 609, the pull wire 714 is pulled in a proximal direction. Accordingly, in some implementations, the sheath 609 can include a plurality of layers that are selected to provide various properties (e.g., stiffness, lubricity, etc.) to the sheath 609 (or portions thereof) and which can extend along varying lengths of the sheath 609.

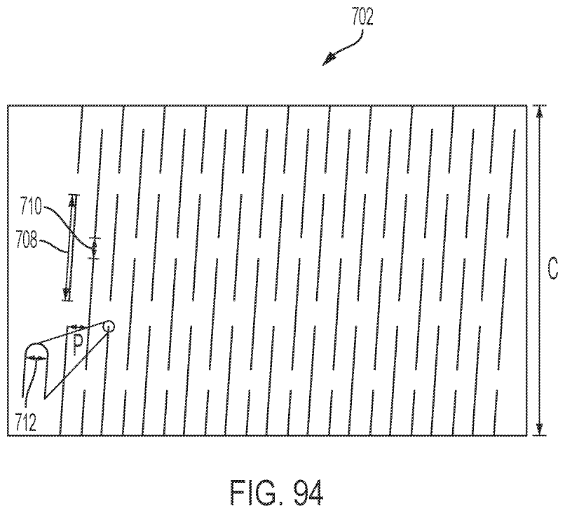

Referring to FIG. 93, in some implementations, a variable stiffness portion can be formed by a single laser-cut hypotube 702 having a variable stiffness along its length. FIG. 93 illustrates four segments (702d, 702e, 702f, 702g) of a laser-cut hypotube, each having a different cut pattern that results in different stiffness in each section. In the illustrated example, the stiffness of the first segment 702d is greater than the stiffness of the second segment 702e, the stiffness of the second segment 702e is greater than the stiffness of the third segment 702f, and the stiffness of the third segment 702f is greater than the stiffness of the fourth segment 702g. In the FIG. 93 example, the uncut area of the first segment 702d is greater than the uncut area of the second segment 702e, the uncut area of the second segment 702e is greater than the uncut area of the third segment 702f, and the uncut area of the third segment 702f is greater than the uncut area of the fourth segment 702g.

The cut patterns of laser cut hypotubes can take a wide variety of different forms. In the example illustrated by FIG. 91, the first segment 702d has a pitch of about 1.27 mm (0.05 inches), 40° cut, and 51° uncut. The second segment 702e has a pitch that transitions from about 1.27 mm (0.05 inches) to about 0.5 mm (0.02 inches), the degrees cut transitions from 400 to 740 cut, and the degrees uncut transitions from 510 to 28.85° uncut. The third segment 702f has a pitch of about 0.5 mm (0.02 inches), 74° cut, and 28.85° uncut. The fourth segment 702g has a pitch that transitions from about 0.5 mm (0.02 inches) to about 0.1 mm (0.004 inches), the degrees cut transitions from 740 to 940 cut, and the degrees uncut transitions from 28.85° to 8.85° uncut. It should be understood that although four segments are illustrated in FIG. 93, the laser-cut hypotube can have any suitable number of segments having different stiffnesses. Additionally, it is contemplated that, in some implementations, the stiffness of the hypotube can decrease gradually in a distal direction (as shown in the second segment 702e and the fourth segment 702g) without defined segments.

The stiffness of each laser-cut hypotube 702 (or a segment of a hypotube) can be selected, for example, based on a material from which the hypotube is formed, a pitch of cuts, degrees of the circumference of the laser-cut hypotube that are cut, degrees of the circumference of the laser-cut hypotube that are uncut, and a kerf width of each cut. In some implementations, the hypotube is formed from stainless steel or nitinol, although other materials are suitable and contemplated.

FIG. 94 illustrates a hypotube 702 having an interrupted spiral cut and shown as a rectangular sheet. In FIG. 94, the circumference of the hypotube is represented by the letter C and the pitch is represented by the letter P. The degrees cut 708 is a length of each cut, and the degrees uncut 710 is a distance between one cut and an adjacent cut in the direction of the circumference C. The kerf width 712 of each cut refers to the width of the space between two adjacent laser cuts at a point at which the sides of the cut are parallel to one another.

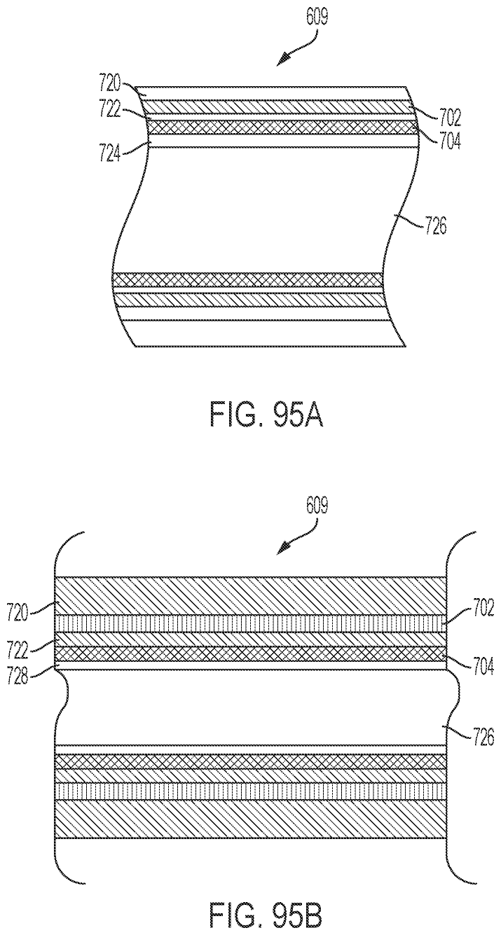

As described above, in some implementations, stiffening materials can be incorporated in the form of a jacket of the sheath 609 of the steerable catheter assembly 608, or the stiffening materials can be incorporated into the structure of the sheath as layers of a multi-layer sheath. Example multi-layer sheaths are shown in FIGS. 95A, 95B, 96A, and 96B. The number and type of layers can vary from what is shown. The layers can be extruded, molded, or otherwise formed and combined in a variety of ways.

FIG. 95A is a longitudinal cross-section of an example multi-layer sheath that includes (from the outer surface of the multi-layer sheath toward the lumen) a first polymer layer 720, a laser-cut hypotube 702, a second polymer layer 722, a braid, mesh, or woven material 704, a third polymer layer 724 defining the lumen 726. Similarly, the multi-layer sheath in FIG. 95B includes (from the outer surface of the multi-layer sheath toward the lumen) the first polymer layer 720, the laser-cut hypotube 702, the second polymer layer 722, the braid, mesh, or woven material 704, and the lumen 726. However, in the example illustrate in FIG. 95B, a polytetrafluoroethylene (PTFE) liner 728 defines the lumen 726. As previously described, the use of a PTFE liner 728 can be used to reduce friction along the internal diameter of the lumen 726. The polymer layers 720, 722, 724 can provide torque resistance based on the durometer of the polymer used, and the laser-cut hypotube 702 and the braid, mesh, or woven material 704 can further provide torque resistance to the multi-layer sheath. It is contemplated that a cross-section of the multi-layer sheath can vary along the length of the sheath. For example, as described herein, the laser-cut hypotube 702, the braid 704, or both can be present in proximal portions of the sheath, but not in distal portions of the sheath to provide a variable stiffness of the sheath.

FIG. 96A illustrates a radial cross-section of an example multi-layer sheath. For example, the cross-section illustrated by FIG. 96A can correspond to one or more portions of the sheath (e.g., to a middle portion of the sheath, etc.) illustrated by FIG. 97. The multi-layer sheath example shown in FIG. 96A includes two lumens. For example, when the multi-layer sheath is used as a sheath 609 of a steerable catheter assembly 608, the shaft 611 of the implant catheter assembly 610 can pass through the lumen 726, and the flex element or flex control element 714, such as the illustrated pull wire, and compression coil 716 can extend through a second lumen, as described above with respect to FIG. 91. In the example shown in FIG. 96A, the lumen 726 is defined by a PTFE liner 728, which is positioned within the second polymer layer 722.

In some implementations, a braid, mesh, or woven material 704 is positioned around the second polymer layer 722 and is positioned within the laser-cut hypotube 702. In some implementations, the first layer of polymer 720 surrounds the laser-cut hypotube 702. Within the second polymer layer 722 and adjacent to the PTFE liner 728, a second liner 730 defines the second lumen, through which the flex control element or flex element 714 (e.g., pull wire, pull suture, tension member, etc.) and compression coil 716 extend. It should be appreciated that other layers and other lumens can be present in some implementations, and the layers can be provided in alternative orders while providing the torque resistance described herein.

FIG. 96B illustrates a radial cross-section of an example multi-layer sheath in which the flex element 714 and coil are on the outside of the proximal portion of the sheath. The cross section can correspond to various locations along the length of the sheath. For example, in some implementations, the cross-section illustrated by FIG. 96B corresponds to a proximal portion of the sheath illustrated by FIG. 97. In some implementations, the second lumen for accommodating the flex element runs less than the full length of the multi-layer sheath. In some implementations, the lumen of the multi-layer sheath varies along the length of the multi-layer sheath and, for example, can transition from a circular cross-section to a non-circular cross-section along the length of the multi-layer sheath. The cross-section illustrated in FIG. 96B can correspond to a proximal portion of the multi-layer sheath relative to the radial cross-section illustrated in FIG. 96A, such as along the line AA in FIG. 91. As shown in FIGS. 96B and 97, the flex element 714 is external to the multi-layer sheath, and the lumen 726 is defined by a PTFE liner 728, which is positioned within the second polymer layer 722.

In some implementations, the braid 704 (or other mesh or woven material), the laser-cut hypotube 702, and the first layer of polymer 720 that surrounds the laser-cut hypotube 702 do not extend along the proximal length of the multi-layer sheath, enabling the flex element 714 to enter the second lumen of the multi-layer sheath as described above. The lumen 726 has a substantially circular cross-section. In some implementations, a groove in the mandrel used to support the PTFE liner 728 and the second polymer layer 722 is filled at a position corresponding to the proximal end of the multi-layer sheath to enable the lumen 726 illustrated in FIG. 96B to be formed. In some of these implementations, reflow of the PTFE and/or the polymer used to form the second polymer layer can create a gradual transition between a circular cross-section and the non-circular cross-section illustrated in FIG. 96A. It should be appreciated that other layers and other lumens can be present in some implementations, and the layers can be arranged in various orders while providing the torque resistance described herein.

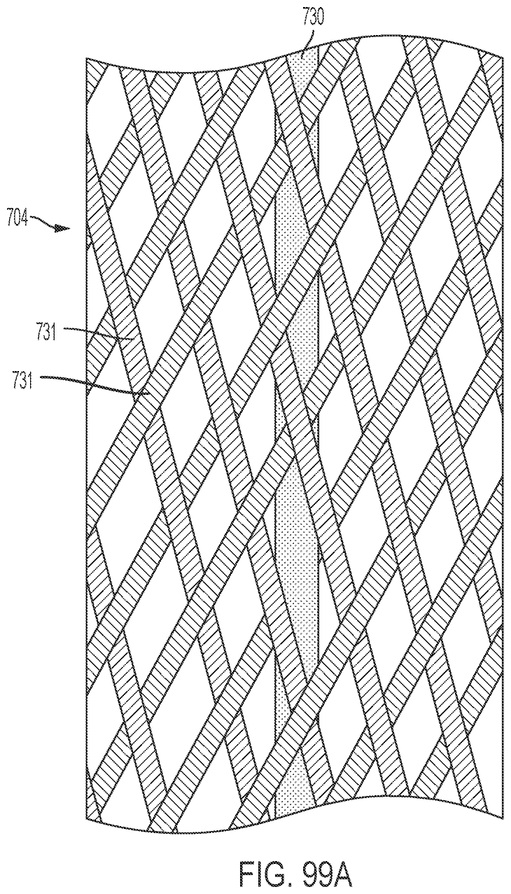

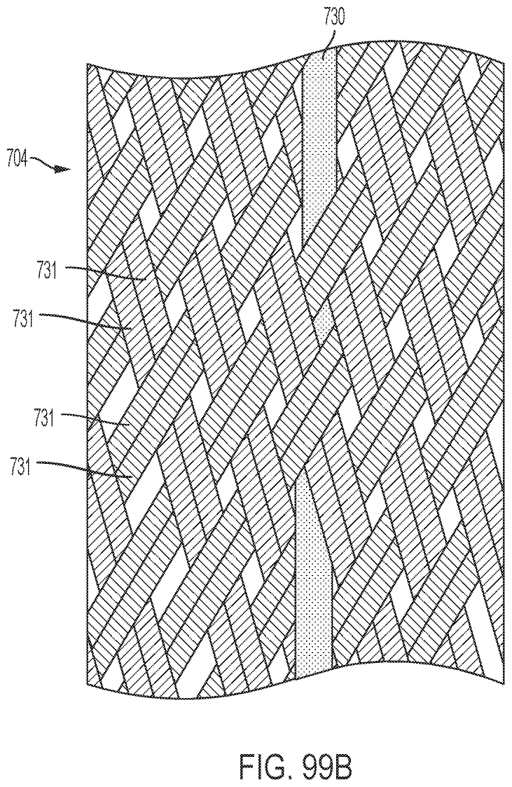

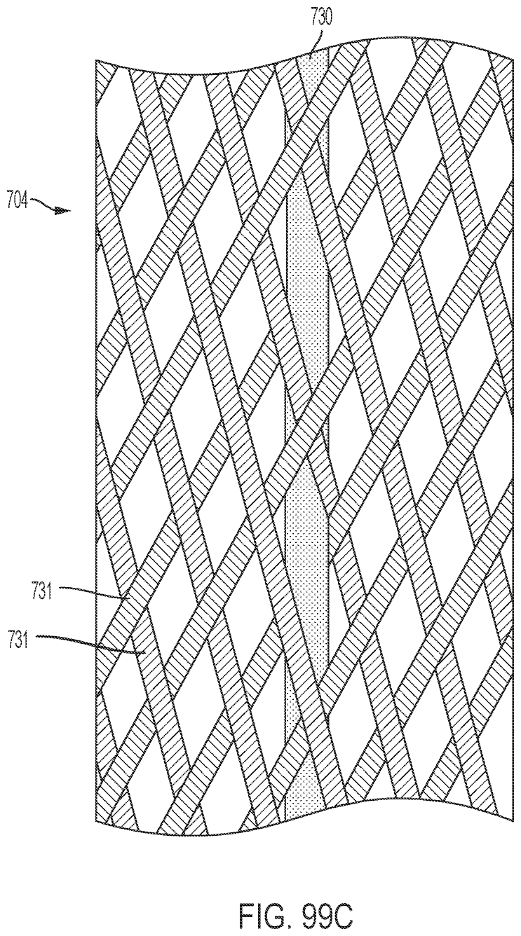

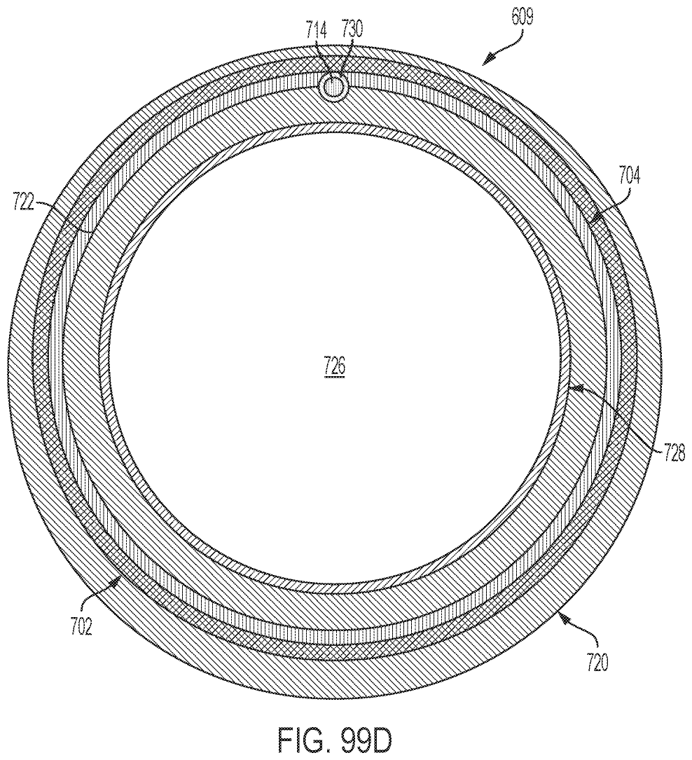

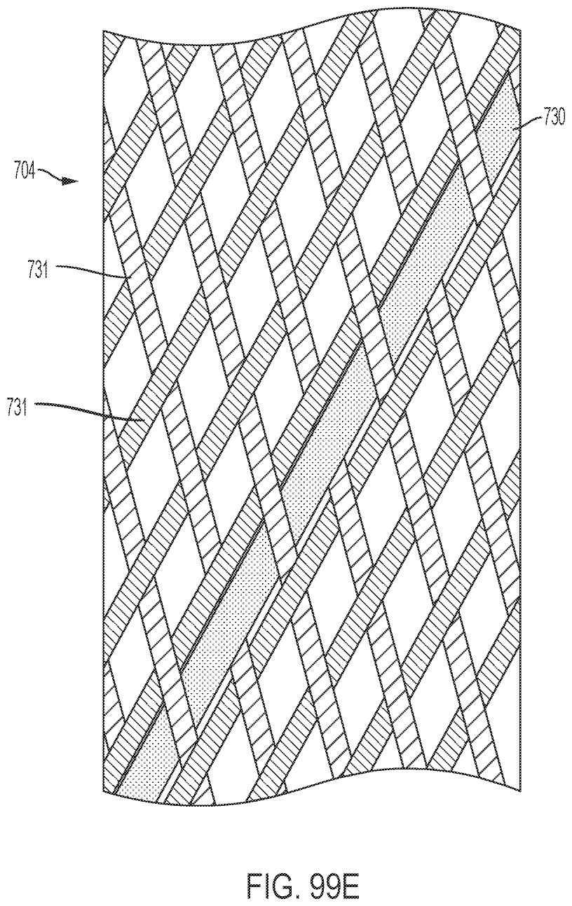

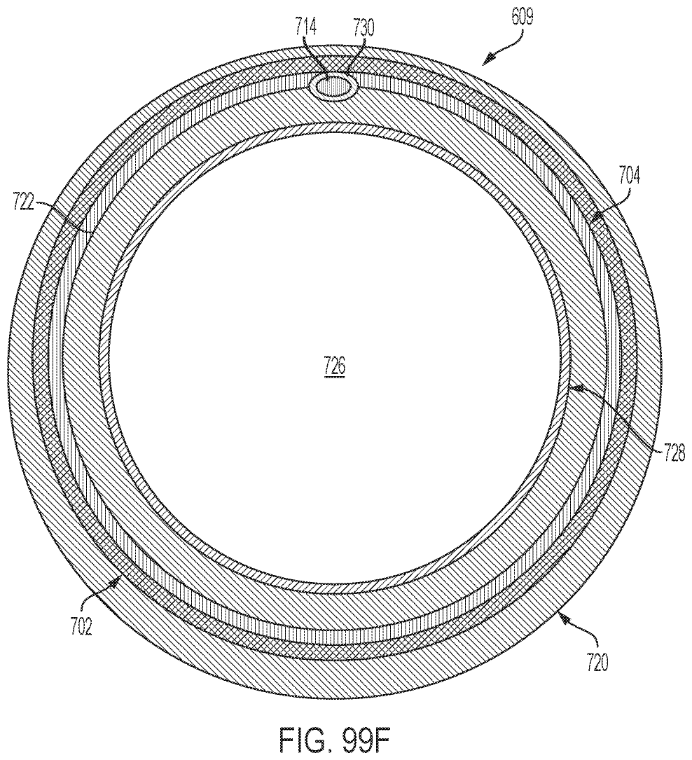

In still further implementations, the braid of the multi-layer sheath can be used to facilitate a passage for the flex control element or flex element (e.g., pull wire, pull suture, tension member, etc.). The incorporation of the passage into the braid can enable the separate lumen (lumen 726) under the braid, and, in some implementations, the compression coil 716, to be removed, which can in turn reduce the outer diameter profile of the multi-layer sheath, allow for a more uniform (e.g., circular) profile, allow for a larger inner profile, and/or reduce manufacturing complexity. Various braid patterns incorporating a flex element passage are illustrated in FIGS. 99A-99C and 99E.

In FIGS. 99A-99C, a lumen 730 is woven into the braid 704, with the lumen 730 extending longitudinally or along the direction of the length of the sheath. The lumen can extend along the length of the sheath in a variety of different ways. For example, the lumen can extend longitudinally and wires 731 of the braid are disposed at two opposing angles relative to the lumen. In some implementations, the lumen and the other wires form a triaxial braid. In some implementations, the lumen 730 and the wires 731 of the braid form sixty degree angles or about sixty degree angles relative to one another.

The lumen 730 can be a tube, such as a thin-walled tube, made from any suitable material. In some implementations, the lumen 730 can be a stainless steel or nitinol hypotube. In some other implementations, the lumen 730 can be a polymeric tube, such as a tube made from polyamide, PEEK, or other polymers known and used in the art. The lumen can be made from any material. In the implementations illustrated in FIGS. 99A-99C, the lumen 730 is incorporated into the braid structure and runs longitudinally along the length of the braid 704. While providing the passage for the flex element (e.g., pull wire, pull suture, tension member, etc.), the lumen 730 can further reinforce the braid and strength of the multi-layer sheath. In some implementations, the lumen 730 is braided in and out of every other crossing or pick of the braid 704, although other patterns are contemplated and possible. As illustrated in FIGS. 99A-99C, the lumen 730 can be incorporated into any one of a variety of braid patterns, such as tri-axial braid patterns or any pattern where the lumen extends longitudinally. For example, FIG. 99A illustrates the longitudinally extending lumen 730 incorporated in a full braid pattern, FIG. 99B illustrates the longitudinally extending lumen 730 incorporated in a diamond braid pattern, and FIG. 99C illustrates the lumen 730 incorporated in a half diamond braid pattern.

FIG. 99D illustrates a radial cross-section of an example multi-layer sheath in which the flex element 714, such as the pull wire illustrated in the figure, passes through a longitudinally extending lumen 730 that is woven into the braid 704. The flex element 714 and the lumen 730 can be used in the same manner as the flex element and the compression coil 716 described above to flex or steer the sheath. For example, application of tension to the flex element 714 in the lumen 730 causes a portion of the sheath to flex. The cross-section can correspond to various locations along the length of the sheath. For example, in some implementations, the cross-section illustrated by FIG. 99D corresponds to a proximal portion of the sheath illustrated by FIG. 97. In some implementations, the lumen 730 for accommodating the flex element runs less than the full length of the multi-layer sheath.

In some implementations, the braid 704 (or other mesh or woven material), the laser-cut hypotube 702, and the first layer of polymer 720 that surrounds the laser-cut hypotube 702 do not extend along the proximal length of the multi-layer sheath, enabling the flex element 714 to enter the lumen 730 of the multi-layer sheath as described above. The lumen 730 can have a substantially circular cross-section. It should be appreciated that other layers and other lumens can be present in some implementations, and the layers can be provided in alternative orders while providing the torque resistance described herein.

In FIG. 99E, the lumen 730 is woven into the braid 704 such that the lumen does not extend only longitudinally or only in the direction of the length of the sheath. For example, the lumen 730 can wrap or twist about the sheath as the lumen extends along the length of the sheath (e.g. in a helical configuration). In some braids, chase wires that extend along or parallel with another wire of the braid are included. In the implementation illustrated by FIG. 99E, the lumen 730 replaces a chase wire in the braid.

As above, the lumen 730 can be a tube, such as a thin-walled tube, made from any suitable material. In some implementations, the lumen 730 can be a stainless steel or nitinol hypotube. In some other implementations, the lumen 730 can be a polymeric tube, such as a tube made from polyamide, PEEK, or other polymers known and used in the art. The lumen 730 can be made from any material. In the implementations illustrated in FIG. 99E, the lumen 730 is braided into the braid 704 next to another wire of the braid. While providing the passage for the flex element, such as the illustrated pull wire, the lumen 730 can further reinforce the braid and strength of the multi-layer sheath. As a chase wire-type feature, the lumen can reduce bowing of the multi-layer sheath during application of tension to the flex element, as it spirals around the multi-layer sheath. In implementations, the lumen 730 is incorporated as a chase wire feature in a half diamond braid pattern. However, the lumen 730 can have any spiral or helical configuration and can be incorporated into any braid pattern.

FIG. 99F illustrates a radial cross-section of an example multi-layer sheath in which the flex element 714 passes through a spiraled, helical, or otherwise winding lumen 730 that is woven into the braid 704. The cross sections of the lumen 730 and flex element 714 are non-circular (e.g. elliptical or otherwise shaped), since the section extends through the spiraled, helical, or otherwise winding flex element, such as the illustrated pull wire 714, and lumen 730. The cross section can correspond to various locations along the length of the sheath. For example, in some implementations, the cross-section illustrated by FIG. 99F corresponds to a proximal portion of the sheath illustrated by FIG. 97. In some implementations, the lumen 730 for accommodating the flex element runs less than the full length of the multi-layer sheath.

In some implementations, the braid 704 (or other mesh or woven material), the laser-cut hypotube 702, and the first layer of polymer 720 that surrounds the laser-cut hypotube 702 do not extend along the proximal length of the multi-layer sheath, enabling the flex element 714 to enter the lumen 730 of the multi-layer sheath as described above. In contrast to the lumen illustrated in FIG. 99D, the lumen 730 illustrated in FIG. 99F has a substantially elliptical cross-section due to its coiled configuration (e.g. not longitudinally extending) within the braid 704. It should be appreciated that other layers and other lumens can be present in some implementations and can be arranged in various orders while providing the torque resistance described herein.

While various inventive aspects, concepts and features of the disclosures may be described and illustrated herein as embodied in combination in the example implementations, these various aspects, concepts, and features may be used in many alternative implementations, either individually or in various combinations and sub-combinations thereof. Unless expressly excluded herein all such combinations and sub-combinations are intended to be within the scope covered herein. Still further, while various alternative implementations as to the various aspects, concepts, and features of the disclosures-such as alternative materials, structures, configurations, methods, devices, and components, alternatives as to form, fit, and function, and so on—may be described herein, such descriptions are not intended to be a complete or exhaustive list of available alternative implementations, whether presently known or later developed. One or more of the inventive aspects, concepts, or features can be adapted into additional implementations and uses within the scope of the present application even if such implementations are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated. Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. Further, the techniques, methods, operations, steps, etc. described or suggested herein can be performed on a living animal or on a non-living simulation, such as on a cadaver, cadaver heart, simulator (e.g., with the body parts, tissue, etc. being simulated), etc. The words used in the claims have their full ordinary meanings and are not limited in any way by the description of the implementations in the specification.

What is claimed is:

1. A delivery system for an implantable device, the delivery system comprising:
a first catheter assembly having a handle and a sheath extending from the handle in an axial direction, the sheath comprising a steerable section;
a second catheter assembly having a handle and a sheath comprising a proximal end coupled to the handle and a distal end;
the sheath of the second catheter assembly extending coaxially through the sheath of the first catheter assembly;
wherein the sheath of the second catheter assembly has a first outer diameter along a first length and a second outer diameter along a second length;
wherein the second outer diameter is smaller than the first outer diameter;
wherein the sheath of the second catheter assembly is extendable from a distal end of the sheath of the first catheter assembly by a stroke distance; and
wherein the second length is greater than or equal to a sum of the stroke distance and a length of the steerable section of the first catheter assembly, such that the sheath of the second catheter assembly has the second outer diameter through the steerable section of the first catheter assembly.

2. The delivery system for an implantable device according to claim 1, wherein a difference between the first outer diameter and the second outer diameter is from about 0.25 to about 0.76 mm.

3. The delivery system for an implantable device according to claim 1, wherein a transition from the first outer diameter to the second outer diameter forms a smooth taper over a distance of from about 25 mm to about 50 mm.

4. The delivery system for an implantable device according to claim 1, further comprising a lubricated coating on an outer surface of the sheath along the second length of the sheath.

5. The delivery system for an implantable device according to claim 4, wherein the lubricated coating comprises a hydrophilic coating.

6. The delivery system for an implantable device according to claim 1, wherein the first outer diameter and the second outer diameter are each less than an inner diameter of the sheath of the first catheter assembly.

7. The delivery system according to claim 1, wherein the first catheter assembly is a steerable catheter assembly, and the second catheter assembly is an implant catheter assembly.

8. A catheter assembly for controlling an implantable device, the catheter assembly comprising:
a handle housing;
a sheath extending distally from the handle housing;
an actuation element extending through the sheath, the actuation element configured to be coupled to one or more paddles of the implantable device;
a control knob coupled to the actuation element that is rotatable relative to the handle housing, wherein rotation of the control knob causes axial movement of the actuation element with respect to the handle housing and the sheath;
an externally threaded retractor coupled to the control knob and the actuation element, wherein the externally threaded retractor is rotationally fixed with respect to the handle housing, and wherein the rotation of the control knob advances the externally threaded retractor in an axial direction, thereby causing linear movement of the actuation element;
a clutch spring that biases the externally threaded retractor distally towards threads of an internally threaded tube within the handle housing;
a pair of clasp actuation lines extending through the sheath, each clasp actuation line of the pair of clasp actuation lines configured to be coupled to a pair of clasps of the implantable device; and
a pair of clasp control members, wherein each clasp control member of the pair of clasp control members is axially movable relative to the handle housing, wherein the axial movement of each of the pair of clasp control members causes one of the pair of clasps to move between an open configuration and a closed configuration.

9. The catheter assembly according to claim 8, wherein the rotation of the control knob causes rotation of the internally threaded tube with respect to the handle housing, which drives the externally threaded retractor to a proximal position in which external threads of the externally threaded retractor and internal threads of the internally threaded tube disengage, and wherein continued rotation of the control knob provides an audible indication.

10. The catheter assembly according to claim 8, further comprising a release element extending from a proximal end of the handle housing and coupled to the actuation element.

11. The catheter assembly according to claim 10, wherein the release element is a release knob comprising an elongated shaft having a plurality of asymmetrical teeth extending from an outer surface of the elongated shaft, wherein the handle housing comprises a ratchet insert comprising one or more pawls configured to contact the outer surface of the elongated shaft, and wherein the plurality of asymmetrical teeth enables the release knob to be rotated in a first direction to withdraw the actuation element into the catheter assembly and inhibits the release knob from being rotated in a second direction.

12. The catheter assembly according to claim 8 further comprising a nose grip having a distal flange at a distal end of the nose grip and a proximal end connected to a distal end of the handle housing, wherein the nose grip comprises a passage extending from the proximal end to the distal end of the nose grip, wherein the distal flange has an outer diameter that is greater than an outer diameter of a central portion of the nose grip.

13. The catheter assembly according to claim 12, further comprising a proximal flange positioned at the proximal end of the nose grip adjacent to the distal end of the handle housing.

14. The catheter assembly according to claim 8, wherein the handle housing comprises a first detent at a first axial position along a path of one of the pair of clasp control members to maintain the one of the pair of clasp control members in a proximal position and a second detent at a second axial position along the path of one of the pair of clasp control members to maintain the one of the pair of clasp control members in a distal position.

15. The catheter assembly according to claim 8, wherein each of the pair of clasp control members extend approximately 50% of the way around a circumference of the handle housing such that together the pair of clasp control members encircle or otherwise surround the circumference of the handle housing.

16. A catheter assembly for a transvascular delivery system comprising:
a handle housing;
a sheath extending longitudinally from the handle housing;
wherein the sheath has a proximal portion and a distal portion;
wherein the sheath is a multi-layer sheath;
wherein one or more layers of the multi-layer sheath comprise at least one laser-cut hypotube;
wherein the at least one laser-cut hypotube has a stiffness that varies along a length of the at least one laser-cut hypotube; and
wherein the proximal portion of the sheath is stiffer than the distal portion of the sheath.

17. The catheter assembly according to claim 16, wherein the multi-layer sheath has a first lumen extending through the sheath, and the proximal portion of the sheath comprises a layer comprising a braid, mesh, or woven material having a material defining a second lumen woven therethrough.

18. The catheter assembly according to claim 17, wherein the second lumen terminates at a location proximal to a steerable portion of the sheath.

19. The catheter assembly according to claim 17, wherein the material defining the second lumen comprises a stainless steel hypotube, a nitinol hypotube, or a polymeric tube.

20. The catheter assembly according to claim 16, wherein the sheath defines a lumen extending longitudinally through the sheath, and wherein the lumen has a cross-section that transitions from having a circular shape to having a non-circular shape.
